SEQUENCE LISTING (142 sequences)

SEQ ID NO: 1 - DNA, 1458 bp, Issatchenkia orientalis
  CDS (1)..(1458)
atgttagctgctagatcattaaaggcaagaatgtcaacaagagctttctcaactacctcaattgcaaaaagaatcgaaaaagatgcatttggtgacattgaagtcccaaatgagaaatattggggtgctcaaactcaaagatctttacaaaatttcaaaattggtggtaagagagaagttatgccagaaccaatcatcaaatcttttggtattttaaagaaggctactgctaagatcaatgctgagtctggtgctttagacccaaagttatctgaagccatccaacaagctgcaaccgaagtttatgaaggtaaactaatggaccatttcccattagttgtctttcaaaccggttctggtactcaatctaacatgaatgccaatgaagtcatctctaatagagcaattgaaatcttgggtggtgaattaggctctaaaactccagtccatcctaatgatcatgttaatatgtcccaatcttctaatgatactttccctactgtcatgcatattgcagcagttacagaagtttcatcccatttattaccagaattaactgcactaagagatgcattgcaaaagaaatccgatgaatttaagaatattatcaaaatcggtagaacccatttacaagatgcaactcctttaactttaggtcaagaattttctggttatgttcaacaatgtactaatggtatcaaaagaatcgaaattgctcttgaacatttgagatacttagctcaaggtggtactgccgttggtactggtcttaacaccaagaaaggttttgctgaaaaggttgcaaatgaagtcactaaattgactggtttacaattctataccgctccaaataaattcgaagcccttgcagctcacgatgctgttgttgaaatgtctggtgctttgaataccgttgcagtctcattattcaaaatcgctcaagatatcagatatttgggttccggcccaagatgtggttatggtgaattggctttaccagaaaatgaaccaggttcttccatcatgccgggtaaagttaacccaactcaaaacgaagctttgactatgctttgtacccaagtctttggtaaccactcttgtattacctttgcaggtgcttcaggtcaattcgaattgaatgtctttaagccagttatgatctccaacttgttatcttctattaggttattaggtgatggttgtaattcttttagaatccactgtgttgaaggtatcattgcaaataccgacaagattgataaattactacatgaatctctcatgttagttactgctttgaacccacacattggttacgataaggcttccaagattgcaaagaatgcacacaagaagggcttgacattgaaacaatctgcattggaattaggttacttgaccgaagaacaattcaatgaatgggttagaccagaaaacatgattggtccaaaggattaa

SEQ ID NO: 2 - protein, 485 aa, Issatchenkia orientalis
MLAARSLKARMSTRAFSTTSIAKRIEKDAFGDIEVPNEKYWGAQTQRSLQNFKIGGKREVMPEPIIKSFGILKKATAKINAESGALDPKLSEAIQQAATEVYEGKLMDHFPLVVFQTGSGTQSNMNANEVISNRAIEILGGELGSKTPVHPNDHVNMSQSSNDTFPTVMHIAAVTEVSSHLLPELTALRDALQKKSDEFKNIIKIGRTHLQDATPLTLGQEFSGYVQQCTNGIKRIEIALEHLRYLAQGGTAVGTGLNTKKGFAEKVANEVTKLTGLQFYTAPNKFEALAAHDAVVEMSGALNTVAVSLFKIAQDIRYLGSGPRCGYGELALPENEPGSSIMPGKVNPTQNEALTMLCTQVFGNHSCITFAGASGQFELNVFKPVMISNLLSSIRLLGDGCNSFRIHCVEGIIANTDKIDKLLHESLMLVTALNPHIGYDKASKIAKNAHKKGLTLKQSALELGYLTEEQFNEWVRPENMIGPKD

SEQ ID NO: 3 - DNA, 2652 bp, Escherichia coli
  CDS (1)..(2652)
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 4 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 5 - DNA, 2643 bp, Mannheimia succiniciproducens
  CDS (1)..(2643)
atgacagaagaatatttaatgatgcgtaataacatcaatatgctggggcgctttttgggcgaaactattcaggaggcgcaaggtgacgatattctcgaactgattgaaaatatccgcgtactgtcccgcaattcccgtagcggcgatgacaaagcccgggcggcattattagacaccctttccactatttcggcggataatattattccggttgcccgcgctttcagccagtttctgaacctgacaaatgtggcggaacaatatcaaaccatgtctcgctcccatgaagataaggtttctgcggaacgttccactgctgcgctgttcgcccgcctgaaagaacaacatgtttctcaggaagaaatcattaaaaccgtacagaaactgttgattgaaatcgtccttaccgctcacccgacggaagttacccgccgttcattaatgcacaaacaggttgaaatcaacaaatgtctggctcagctggatcatacggatttaaccgccgaagaacaaaaaaatattgagtataaattacttcgtcttatcgccgaagcctggcataccaatgaaatccgtaccaatcggccgacacctctggaagaagccaaatggggttttgccgttatcgaaaacagtttatgggaaggtttgcccgcctttatccgcaaacttaacgatgccgccgtcgaacatttaaattatgctttgccggtagacctcacaccggtacgcttctcttcctggatgggcggtgaccgtgacggcaaccccttcgttaccgcaaaaattacccgggaagcgctgcaacttgcgcgctggaaagcggcggatttatttttaaccgatattcaggaactctgcgacgagttgtcaatgacacaatgcactgcggaattccgagaaaaatacggtgatcatttagaaccctatcgtgtagttgtgaaggatttacgcagcaaattaaaaaatacgctggattattacaacgatatacttgcgggtcgcattccgccgtttaaacaagatgaaatcatcagtgaagaccaacaactctggcaaccgctttatgactgttatcaatccctaaccgcctgcggtatgcgtattattgccaatggattattgctggataccttacgccgcgttcgttgtttcggcgtcacattactgcgtttagatatccgtcaggaaagcacccgccatagcgacgccatcggcgaaattacccgctacatcggtttaggcgattacagccaatggacagaagatgacaaacaagccttcctgatccgggaattaagttcccgtcgtccgctaattccccataactggacgccttcggaacacactcgggaaattttagacacctgtaaagtcattgcaaaacagccggaaggcgttatttcctgctatatcatttccatggcgcgcaccgcttccgatgttttggcggtgcatttattattgaaagaagcgggcatttcataccatctgccggtagttcctctatttgaaacattggacgacctggacgcttctaaagaagtgatgacgcaactgtttaacgtaggctggtatcgcggcgtaatcaaaaaccgccaaatgatcatgatcggctattccgatagcgccaaagatgcgggcatgatggcggcctcatgggcgcaataccgggcgcaggacgctttagtcaaactttgcgaacaaaccggcatcgaacttaccctcttccacggccgcggcggcaccgtaggacgtggcggtgcaccggctcacgccgcattattatcccaaccgccacgttctctgaaaaacggcttacgggtaaccgaacaaggggaaatgatccgcttcaaactgggattaccggctatcgccgcagaaagtctggatctctacgccagcgccattcttgaggccaacctcctgccgccgccggaaccgaaagccagctggtgccgggtaatggacgaacttgccgtcgcttcttgcgaaatctatcgcaatgtggtgcgcggcgataaagattttgtgccttacttccgcagcgccacaccggaacaggaactggcaaaactgcctttaggttcccgaccggcaaaacgcaatccgaacggcggcgttgaaagcctgcgtgccattccctggatcttcgcctggatgcaaaaccgcctgatgctgcccgcctggctcggtgccggcgcctcaatccgtcaggcgatggaaagcggcaaagcggcggtgattgaagaaatgtgcaaccattggccgtttttcaatacccgaatcggcatgcttgaaatggtattcagtaaaaccgatagctggctgtccgaatattacgaccagcgtttagtgaaaaaagagctttggtatttaggcgaatcgctgcgcaaacagttaagcgaagatatcgctaccgtgttacggctttccggcaaaggcgatcaattaatgtcggatttgccttgggtggcggaatctattgcactgcgtaacgtttacaccgacccgttaaacttattgcaagtggaattattgcgtcgtttgcgagcggatcccgaacatccgaatccggatatcgagcaagcgctgatgatcaccattaccggtatcgccgcgggtatgcgtaatacgggttag

SEQ ID NO: 6 - protein, 880 aa, Mannheimia succiniciproducens
MTEEYLMMRNNINMLGRFLGETIQEAQGDDILELIENIRVLSRNSRSGDDKARAALLDTLSTISADNIIPVARAFSQFLNLTNVAEQYQTMSRSHEDKVSAERSTAALFARLKEQHVSQEEIIKTVQKLLIEIVLTAHPTEVTRRSLMHKQVEINKCLAQLDHTDLTAEEQKNIEYKLLRLIAEAWHTNEIRTNRPTPLEEAKWGFAVIENSLWEGLPAFIRKLNDAAVEHLNYALPVDLTPVRFSSWMGGDRDGNPFVTAKITREALQLARWKAADLFLTDIQELCDELSMTQCTAEFREKYGDHLEPYRVVVKDLRSKLKNTLDYYNDILAGRIPPFKQDEIISEDQQLWQPLYDCYQSLTACGMRIIANGLLLDTLRRVRCFGVTLLRLDIRQESTRHSDAIGEITRYIGLGDYSQWTEDDKQAFLIRELSSRRPLIPHNWTPSEHTREILDTCKVIAKQPEGVISCYIISMARTASDVLAVHLLLKEAGISYHLPVVPLFETLDDLDASKEVMTQLFNVGWYRGVIKNRQMIMIGYSDSAKDAGMMAASWAQYRAQDALVKLCEQTGIELTLFHGRGGTVGRGGAPAHAALLSQPPRSLKNGLRVTEQGEMIRFKLGLPAIAAESLDLYASAILEANLLPPPEPKASWCRVMDELAVASCEIYRNVVRGDKDFVPYFRSATPEQELAKLPLGSRPAKRNPNGGVESLRAIPWIFAWMQNRLMLPAWLGAGASIRQAMESGKAAVIEEMCNHWPFFNTRIGMLEMVFSKTDSWLSEYYDQRLVKKELWYLGESLRKQLSEDIATVLRLSGKGDQLMSDLPWVAESIALRNVYTDPLNLLQVELLRRLRADPEHPNPDIEQALMITITGIAAGMRNTG

SEQ ID NO: 7 - DNA, 3543 bp, Issatchenkia orientalis
  CDS (1)..(3543)
atgtcaactgtggaagatcactcctccctacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagactggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacgggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaacggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagttttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcgttttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacctctcaaccttcaatgagtgcttttattgctgctttagatggtgatatcgaaactggtgttccagaacattttgcaagacaattagatgcatactgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggctcctccaactattggtgaagaagttcatgtggaaattgagcaaggtaagactttgattattaagttattagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaa

SEQ ID NO: 8 - protein, 1180 aa, Issatchenkia orientalis
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVREFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 9 - DNA, 3537 bp, Saccharomyces cerevisiae
  CDS (1)..(3537)
atgtcgcaaagaaaattcgccggcttgagagataacttcaatctcttgggtgaaaagaacaaaatattggtggctaatagaggagaaattccaatcagaatttttcgtaccgctcatgaactgtctatgcagacggtagctatatattctcatgaagatcgtctttcaacgcacaaacaaaaggctgacgaagcatacgtcataggtgaagtaggccaatatacccccgtcggcgcttatttggccattgacgaaatcatttccattgcccaaaaacaccaggtagatttcatccatccaggttatgggttcttgtctgaaaattcggaatttgccgacaaagtagtgaaggccggtatcacttggattggccctccagctgaagttattgactccgtgggtgataaggtctcagctagaaacctggcagcaaaagctaatgtgcccaccgttcctggtacaccaggtcctatagaaactgtagaggaagcacttgacttcgtcaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggccgataaccacggaaacgtggttcatcttttcgaaagagactgttccgtgcagagaagacaccaaaaggttgtcgaagtggccccagcaaagactttaccccgtgaagtccgtgacgccattttgacagatgcagttaaattggccaaagagtgtggctacagaaatgcgggtactgctgaattcttggttgataaccaaaatagacactatttcattgaaattaatccaagaatccaagtggaacataccatcacagaagaaattaccggtatagatattgtggcggctcagatccaaattgcggcaggtgcctctctaccccagctgggcctattccaggacaaaattacgactcgtggctttgccattcagtgccgtattaccacggaagaccctgctaagaacttccaaccagataccggtagaatagaagtgtaccgttctgcaggtggtaatggtgttagactggatggtggtaacgcctatgcaggaacaataatctcacctcattacgactcaatgctggtcaaatgctcatgctccggttccacctacgaaatcgttcgtagaaaaatgattcgtgcattaatcgagttcagaattagaggtgtcaagaccaacattcccttcctattgactcttttgaccaatccagtatttattgagggtacatactggacgacttttattgacgacaccccacaactgttccaaatggtttcatcacaaaacagagcccaaaaacttttacattacctcgccgacgtggcagtcaatggttcatctatcaagggtcaaattggcttgccaaaattaaaatcaaatccaagtgtcccccatttgcacgatgctcagggcaatgtcatcaacgttacaaagtctgcaccaccatccggatggaggcaagtgctactagaaaaggggccagctgaatttgccagacaagttagacagttcaatggtactttattgatggacaccacctggagagacgctcatcaatctctacttgcaacaagagtcagaacccacgatttggctacaatcgctccaacaaccgcacatgcccttgcaggtcgtttcgccttagaatgttggggtggtgccacattcgatgttgcaatgagatttttgcatgaggatccatgggaacgtttgagaaaattaagatctctggtgcctaatattccattccaaatgttattgcgtggtgccaatggtgtggcttattcttcattgcctgacaatgctattgaccatttcgtcaagcaagccaaggataatggtgttgatatatttagagtctttgatgccttaaatgacttggaacaattgaaggtcggtgtagatgctgtgaagaaggcaggtggtgttgtagaagccactgtttgtttctctggggatatgcttcagccaggcaagaaatacaatttggattactacttggaaattgctgaaaaaattgtccaaatgggcactcatatcctgggtatcaaagatatggcaggtaccatgaagccagcagctgccaaactactgattggatctttgagggctaagtaccctgatctcccaatacatgttcacactcacgattctgcaggtactgctgttgcatcaatgactgcgtgtgctctggcgggcgccgatgtcgttgatgttgccatcaactcaatgtctggtttaacttcacaaccatcaatcaatgctctgttggcttcattagaaggtaatattgacactggtattaacgttgagcatgtccgtgaactagatgcatattgggcagagatgagattgttatactcttgtttcgaggctgacttgaagggcccagatccagaagtttatcaacatgaaatcccaggtggtcaattgacaaacttgttgtttcaagcccaacaattgggtcttggagaacaatgggccgaaacaaaaagagcttacagagaagccaattatttattgggtgatattgtcaaagttaccccaacttcgaaggtcgttggtgatctggcacaatttatggtctccaataaattaacttccgatgatgtgagacgcctggctaattctttggatttccctgactctgttatggatttcttcgaaggcttaatcggccaaccatacggtgggttcccagaaccatttagatcagacgttttaaggaacaagagaagaaagttgacttgtcgtccaggcctggaactagagccatttgatctcgaaaaaattagagaagacttgcagaatagatttggtgatgttgatgagtgcgacgttgcttcttataacatgtacccaagagtttatgaagacttccaaaagatgagagaaacgtatggtgatttatctgtattgccaacaagaagctttttgtctccactagagactgacgaagaaattgaagttgtaatcgaacaaggtaaaacgctaattatcaagctacaggctgtgggtgatttgaacaaaaagaccggtgaaagagaagtttactttgatttgaatggtgaaatgagaaaaattcgtgttgctgacagatcacaaaaagtggaaactgttactaaatccaaagcagacatgcatgatccattacacattggtgcaccaatggcaggtgtcattgttgaagttaaagttcataaaggatcactaataaagaagggccaacctgtagccgtattaagcgccatgaaaatggaaatgattatatcttctccatccgatggacaagttaaagaagtgtttgtctctgatggtgaaaatgtggactcttctgatttattagttctattagaagaccaagttcctgttgaaactaaggcataa

SEQ ID NO: 10 - protein, 1178 aa, Saccharomyces cerevisiae
MSQRKFAGLRDNFNLLGEKNKILVANRGEIPIRIFRTAHELSMQTVAIYSHEDRLSTHKQKADEAYVIGEVGQYTPVGAYLAIDEIISIAQKHQVDFIHPGYGFLSENSEFADKVVKAGITWIGPPAEVIDSVGDKVSARNLAAKANVPTVPGTPGPIETVEEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKECGYRNAGTAEFLVDNQNRHYFIEINPRIQVEHTITEEITGIDIVAAQIQIAAGASLPQLGLFQDKITTRGFAIQCRITTEDPAKNFQPDTGRIEVYRSAGGNGVRLDGGNAYAGTIISPHYDSMLVKCSCSGSTYEIVRRKMIRALIEFRIRGVKTNIPFLLTLLTNPVFIEGTYWTTFIDDTPQLFQMVSSQNRAQKLLHYLADVAVNGSSIKGQIGLPKLKSNPSVPHLHDAQGNVINVTKSAPPSGWRQVLLEKGPAEFARQVRQFNGTLLMDTTWRDAHQSLLATRVRTHDLATIAPTTAHALAGRFALECWGGATFDVAMRFLHEDPWERLRKLRSLVPNIPFQMLLRGANGVAYSSLPDNAIDHFVKQAKDNGVDIFRVFDALNDLEQLKVGVDAVKKAGGVVEATVCFSGDMLQPGKKYNLDYYLEIAEKIVQMGTHILGIKDMAGTMKPAAAKLLIGSLRAKYPDLPIHVHTHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDVRRLANSLDFPDSVMDFFEGLIGQPYGGFPEPFRSDVLRNKRRKLTCRPGLELEPFDLEKIREDLQNRFGDVDECDVASYNMYPRVYEDFQKMRETYGDLSVLPTRSFLSPLETDEEIEVVIEQGKTLIIKLQAVGDLNKKTGEREVYFDLNGEMRKIRVADRSQKVETVTKSKADMHDPLHIGAPMAGVIVEVKVHKGSLIKKGQPVAVLSAMKMEMIISSPSDGQVKEVFVSDGENVDSSDLLVLLEDQVPVETKA

SEQ ID NO: 11 - DNA, 3528 bp, Kluyveromyces marxianus
  CDS (1)..(3528)
atgtctacccaaaacgatctggccgggttgcgtgataactcgaacctattaggtgaaaagaacaagattcttgttgccaaccgtggtgaaattccaattagaatctttagaacggctcatgaactttcgatgaagactgttgcgatctattcgcacgaggatagactatctatgcacagattgaaggcagacgaagcttacgttattggtgagccaggaaaatacactccagttggtgcgtatttggcgatcgatgagattatcaagattgctcaattgcacggagtgagcttcatccaccctggttatgggttcttatcggaaaactctgagtttgccaagaaggtggccgactctggtatcacgtgggttggtcctccagccgatgtgatcgatgctgttggtgacaaggtttctgccagaaacttggccgagagagcggatgttccagtggttccaggtacgcctggtccaatagagacagttgaagaagcagttgaatttgtggagaagtacggatacccagtcatcatcaaggctgccttcggtggtggtggtcgtggtatgagagttgttcgtgaaggtgatgatatcgccgatgctttccaaagagccaagtccgaagctgttactgctttcggtaacggtacttgtttcgttgaaagattcttggacaagccaaagcacatcgaagttcagttgttggctgatcactacggtaatgtcatccatctattcgaaagagactgttctgtgcaaagaagacatcaaaaggtcgttgaagtagcgccagccaagactttgccagagagcgtgcgtaatgcaatcttgactgacgctgtcaagttggctaaggaggcaggatacagaaatgctggtaccgctgaatttttggtcgacaaccaaaacagacactactttattgaaatcaacccaagaattcaagtcgaacataccatcaccgaagaaattaccggtatcgacattgtcgccgcacaaattcaaatcgcagcaggtgcttccttggaacaattgggactattgcaagatagaatcaccacccgtggtttcgctattcaatgtcgtatcactactgaagatccttccaagaacttccagccagatactggtcgtatcgatgtttaccgttccgctggtggtaacggtgtcagattggatggtggtaacgcattcgctggttcggtcatttcacctcattatgattccatgttggtcaaatgttcttgttccggttccacttacgaaatcgttcgtcgtaagatgttgcgtgccttgatcgaattcagaatcagaggtgtgaagacaaacattccattcttgctaacgttgttgactcatcctgtgttcaagtccggtgactactggactaccttcatcgatgacactccacaattgttcgaaatggtttcttctcaaaacagagcacaaaaactattgcactacttggccgatcttgccgttaacggttcatcgatcaagggtcaaattggtctaccaaagttaaagactcatcctactatcccacatttgcataaggccgatggctccattctagatgtgtctgccaagcctcctgccgggtggagagatgttctattgcaacacggcccagaagaatttgcaaagcaagttagaaagttcaagggtactttgctaatggacaccacctggagagatgctcatcaatctctattggccactagagtcagaacttacgatttggctgccatcgctccaactactgctcatgctttgagcggtgctttcgctttggaatgttggggtggtgccactttcgatgtctccatgagattcttgcacgaagatccatgggaacgtttgagaactttgagaaagttggttcctaacattccattccaaatgttgctacgtggtgccaacggtgttgcatactcttctctaccagataacgctatcgaccactttgtcaagcaagcaaaggataacggtgttgacattttcagagtcttcgatgctctaaacgatttggagcaattgactgtcggtgttgacgctgtcaagaaggctggtggtgttgtcgaagctaccatttgttactccggtgacatgctagcaccaggtaagaagtacaaccttgactactacttggacattgttgaacaagtggttaagagaggtacccatattcttggtatcaaggatatggcaggtactttgaagccatctgctgctaagctcttgatcggttctatcagaacaaagtaccctgacttgccaattcacgtccatacccatgactccgccggtaccggtgttgcttccatggctgcatgtgctttcgctggtgctgatgttgttgatgttgcaaccaactctatgtctggtatgacttctcaaccatctgtcaatgcactattggctgctcttgatggtgaaatcgactgtaatgtcaacgtcagctacatcagtcagctagatgcttactgggctgaaatgagactattgtactcatgtttcgaagccgacttgaagggtcctgatccagaagtttacgtccatgaaattccaggtggtcaattgaccaacttgctcttccaagcccaacaattgggtcttggtgagcaatgggctgaaaccaagagagcttaccgtgaagcaaacctgttgttgggtgatgttgttaaggtcactccaacatccaaggttgtcggtgatttggctcaattcatggtcactaacaagttgacctcggatgatgttaagagattagcttcatctttggatttcccagactccgtcatggacttctttgaaggtttaatcggtcaaccatacggtggtttcccagaacctctaagatctgatgttttgaagaacaagagaagaaagttgaccaagagaccaggtttggaattggctccattcgatttggaaggcattaaggaagatttgactaacagatttggtgacattgacgactgtgatgttgcttcttacaacatgtatccaaaggtctacgaagatttccgtaagatcagagaaaagtacggtgatctatctgttttgccaaccaagaacttcttgtctccaccttcaatcggtgaagaaatcgtcgttacaattgaacaaggtaagactttgatcattaagccacaagctattggtgatttgaacaaggagactggtatcagagaagtttacttcgaattgaacggtgaattgagaaaggtctctgttgctgacagatctcaaaaggttgaaacgatctccaagccaaaggctgacgcccacgatccattccaagttggttctccaatggcaggtgttgttgtcgaagtcaaggtacacaagggttctttgatctccaagggccaaccagtcgctgtcctaagtgccatgaagatggaaatggttatctcctccccatctgatggtcaagtcaaggaagtgcttgtcaaggatggtgaaaacgttgacgcttctgacttgctcgttgttttggaagaagctccagctaaagaataa

SEQ ID NO: 12 - protein, 1175 aa, Kluyveromyces marxianus
MSTQNDLAGLRDNSNLLGEKNKILVANRGEIPIRIFRTAHELSMKTVAIYSHEDRLSMHRLKADEAYVIGEPGKYTPVGAYLAIDEIIKIAQLHGVSFIHPGYGFLSENSEFAKKVADSGITWVGPPADVIDAVGDKVSARNLAERADVPVVPGTPGPIETVEEAVEFVEKYGYPVIIKAAFGGGGRGMRVVREGDDIADAFQRAKSEAVTAFGNGTCFVERFLDKPKHIEVQLLADHYGNVIHLFERDCSVQRRHQKVVEVAPAKTLPESVRNAILTDAVKLAKEAGYRNAGTAEFLVDNQNRHYFIEINPRIQVEHTITEEITGIDIVAAQIQIAAGASLEQLGLLQDRITTRGFAIQCRITTEDPSKNFQPDTGRIDVYRSAGGNGVRLDGGNAFAGSVISPHYDSMLVKCSCSGSTYEIVRRKMLRALIEFRIRGVKTNIPFLLTLLTHPVFKSGDYWTTFIDDTPQLFEMVSSQNRAQKLLHYLADLAVNGSSIKGQIGLPKLKTHPTIPHLHKADGSILDVSAKPPAGWRDVLLQHGPEEFAKQVRKFKGTLLMDTTWRDAHQSLLATRVRTYDLAAIAPTTAHALSGAFALECWGGATFDVSMRFLHEDPWERLRTLRKLVPNIPFQMLLRGANGVAYSSLPDNAIDHFVKQAKDNGVDIFRVFDALNDLEQLTVGVDAVKKAGGVVEATICYSGDMLAPGKKYNLDYYLDIVEQVVKRGTHILGIKDMAGTLKPSAAKLLIGSIRTKYPDLPIHVHTHDSAGTGVASMAACAFAGADVVDVATNSMSGMTSQPSVNALLAALDGEIDCNVNVSYISQLDAYWAEMRLLYSCFEADLKGPDPEVYVHEIPGGQLTNLLFQAQQLGLGEQWAETKRAYREANLLLGDVVKVTPTSKVVGDLAQFMVTNKLTSDDVKRLASSLDFPDSVMDFFEGLIGQPYGGFPEPLRSDVLKNKRRKLTKRPGLELAPFDLEGIKEDLTNRFGDIDDCDVASYNMYPKVYEDFRKIREKYGDLSVLPTKNFLSPPSIGEEIVVTIEQGKTLIIKPQAIGDLNKETGIREVYFELNGELRKVSVADRSQKVETISKPKADAHDPFQVGSPMAGVVVEVKVHKGSLISKGQPVAVLSAMKMEMVISSPSDGQVKEVLVKDGENVDASDLLVVLEEAPAKE

SEQ ID NO: 13 - DNA, 1029 bp, Issatchenkia orientalis
  CDS (1)..(1029)
atgtccaatgttaaagtagctctactaggtgccgctggtggtatcggccaaccacttgctctattacttaagcttaatccaaacataacccatttggcactctatgacgttgtgcatgttcctggagtggctgccgacctacaccatatagacacagatgtagtgattacccaccatttgaaagatgaagacggtacggccttggcaaacgccctcaaggacgctacgtttgttattgtccccgccggtgttccgagaaagcccggcatgactagaggtgatttgttcacaattaatgccggtatatgtgccgaattggctaatgctattagtttgaacgctcctaatgcattcacccttgtcattaccaatccggtcaactcgaccgttcctatatttaaggaaatatttgctaaaaatgaagccttcaatccaaggagactgtttggtgtaactgctctagatcatgttagatcaaatacttttctctcggaattaattgacggtaaaaatccccaacattttgatgtcactgttgttggcggacactctggtaactcaattgtccccctattctcccttgttaaggctgccgaaaatttagacgatgaaattatagatgccttgattcatagagttcaatacggtggagatgaagttgtggaagcaaagagcggtgcgggctcggcaactctttcaatggcttatgccgctaacaagttcttcaatatattgcttaatggatacttgggtttgaagaagacaatgatttcaagttatgtctttttagacgattcaatcaacggcgtccctcaattaaaggaaaatttgtctaaacttttgaaaggttccgaggttgagttaccaagttatttggctgttccaatgacctatggtaaagaaggtattgaacaagtcttttacgattgggtgtttgaaatgtcaccaaaggaaaaggaaaacttcattacagcgattgaatacattgatcaaaatattgaaaaaggtctgaattttatggtacgttaa

SEQ ID NO: 14 - protein, 342 aa, Issatchenkia orientalis
MSNVKVALLGAAGGIGQPLALLLKLNPNITHLALYDVVHVPGVAADLHHIDTDVVITHHLKDEDGTALANALKDATFVIVPAGVPRKPGMTRGDLFTINAGICAELANAISLNAPNAFTLVITNPVNSTVPIFKEIFAKNEAFNPRRLFGVTALDHVRSNTFLSELIDGKNPQHFDVTVVGGHSGNSIVPLFSLVKAAENLDDEIIDALIHRVQYGGDEVVEAKSGAGSATLSMAYAANKFFNILLNGYLGLKKTMISSYVFLDDSINGVPQLKENLSKLLKGSEVELPSYLAVPMTYGKEGIEQVFYDWVFEMSPKEKENFITAIEYIDQNIEKGLNFMVR

SEQ ID NO: 15 - DNA, 1011 bp, Issatchenkia orientalis
  CDS (1)..(1011)
atggtcaaggtgactattttaggcgctgccggtggaattggacaaccactctcattgttattgagacttaatccatggattgacgaattggccttgtttgatattgtcaatacccccggcgtgagttgtgatttgtcgcatattcctgcatcacaggttgttaatggctatgctccgaaatcgaaatcagatacagagacaatcaagactgccttgaaaggtgctgatattgttgttattcctgcaggaattccacgtaaacctggtatgacaagaaacgatctctttaaaatcaatgccggaatcgttaagagtttgattcatagtgcaggaaccacttgccctgatgcatttatttgtgtcatttcgaaccctgtcaactcgacagttccaattgccgttgaagaactaaagcgtttgaatgtttttaatccacataaagttttcggtattaccacattggacaatttcagattagaagaatttctgagtggagaacttggtggaattgtcaaaccaaatgatttatatggtgatgtagttgctataggtggccattcgggcgactctatagtaccgatcttgaattcgtggaatttgaatttcatcaatgatggagattcttataacaatttggtcaagagggtccagtttggaggcgatgaggttgtcaaggcaaaggacgggaaaggttcggctacattgtcaatggctacagctgcatacaggtttgtcaacaacctcttggacgccattgtcaataacaagaaagtcaaggaagtggcctttgtgaaaatcgaccaattgccaactacaagggttccttattttgttgttgatgaaactcagtattttagtctacccattattctcggtagacaggggattgagagggtcacgttcccagaatctctgacagagcaagaggtgagaatgacaaagcacgctgttgctaaagttaaagttgacgttaataaaggcttcaattttgtccatggcccaaaactgtaa

SEQ ID NO: 16 - protein, 336 aa, Issatchenkia orientalis
MVKVTILGAAGGIGQPLSLLLRLNPWIDELALFDIVNTPGVSCDLSHIPASQVVNGYAPKSKSDTETIKTALKGADIVVIPAGIPRKPGMTRNDLFKINAGIVKSLIHSAGTTCPDAFICVISNPVNSTVPIAVEELKRLNVFNPHKVFGITTLDNFRLEEFLSGELGGIVKPNDLYGDVVAIGGHSGDSIVPILNSWNLNFINDGDSYNNLVKRVQFGGDEVVKAKDGKGSATLSMATAAYRFVNNLLDAIVNNKKVKEVAFVKIDQLPTTRVPYFVVDETQYFSLPIILGRQGIERVTFPESLTEQEVRMTKHAVAKVKVDVNKGFNFVHGPKL

SEQ ID NO: 17 - DNA, 1005 bp, Issatchenkia orientalis
  CDS (1)..(1005)
atgttctccagaatctctgctagacaattctcctcctctgctgcttccgcttacaaggtcaccgttttaggtgctgcaggtggtattggccaaccactatctcttttgatgaagttgaaccacaaggtcaccaacttatccttgtacgacttgagattgggtgctggtgttgccactgacttgtcccacattccaaccaactccgttgtcaagggctatggtccagaaaacaatggtttgaaggacgccttgaccggctccgatgttgttcttattccagctggtgttccaagaaaaccaggtatgactagagacgatctcttcaacaccaatgcatcgattgtcagagacttggcaaaggctgctgcagaccactgtccaaacgccgtcttgttgatcatttcaaaccctgtcaactcaactgtcccaattgttgctgaggttttgaaatcaaagggcgtctacaacccaaagaagttgtttggtgtcaccactttggacgttttgagatcctcgagattcttgagtgaagtcgtcaacaccgacccaaccaccgaaaccgtcactgttgttggtggccactctggtgtcaccattgttcctttaatctcccaaaccaaacacaaggacttgccaaaggaaacctacgaagcattggtccacagaatccaattcggtggtgatgaggttgtcaaggccaaggacggtgcaggttccgctaccttgtccatggcccaagccggtgcaagaatggcctcctccgtcttgaagggtttggctggtgaagttgacattgtcgaaccaacctttattgactctccattgttcaagtccgaaggtgtcgaattcttctcctccagagtcacccttggtccagaaggtgtccaagaagtccacccattgggcgtcttatctactgctgaagaagaaatggttgctactgctaaggaaaccttgaagaagaacatccaaaagggtgtcgactttgtcaaggctaacccataa

SEQ ID NO: 18 - protein, 334 aa, Issatchenkia orientalis
MFSRISARQFSSSAASAYKVTVLGAAGGIGQPLSLLMKLNHKVTNLSLYDLRLGAGVATDLSHIPTNSVVKGYGPENNGLKDALTGSDVVLIPAGVPRKPGMTRDDLFNTNASIVRDLAKAAADHCPNAVLLIISNPVNSTVPIVAEVLKSKGVYNPKKLFGVTTLDVLRSSRFLSEVVNTDPTTETVTVVGGHSGVTIVPLISQTKHKDLPKETYEALVHRIQFGGDEVVKAKDGAGSATLSMAQAGARMASSVLKGLAGEVDIVEPTFIDSPLFKSEGVEFFSSRVTLGPEGVQEVHPLGVLSTAEEEMVATAKETLKKNIQKGVDFVKANP

SEQ ID NO: 19 - DNA, 1017 bp, Kluyveromyces marxianus
  CDS (1)..(1017)
atgcttagagccctaactcgccgtcaattttcctccactgccttcaacccatacaaggtcaccgttctaggtgctggtggtggtattggtcaaccattgtccttgttgttgaagctaaaccacaaggtcactgacttgagactatacgacttgaagggtgccaagggtgtcgctgctgacttgtctcacatcccaaccaactctaccgttactggttacactccagaatccaaggactctcaagaagaattggctgctgctttgaaggacactgaggttgttttgatcccagctggtgtgccaagaaagccaggtatgacccgtgacgatttgttcgccatcaatgccggtattgtcagagatttggccacttccatcgccaagaacgctccaaacgccgccatcttggtcatctccaacccagtcaactctactgtcccaatcgtcgccgaggtcttgaagcaaaacggcgtctacaacccaaagaagttgttcggtgtcaccactttggacgttatccgtgcctccagattcatctccgaggttagaggtaccgacccaaccactgagcacgtgaccgtcgtcggtggtcactccggtatcaccatcttgccgctagtgtcccagaccaagcacaagtccgtcatcaagggcgaggaattggacaacttgatccacagaatccaattcggtggtgacgaagtcgtccaggcaaagaacggtgctggttctgccactttgtccatggcccaagccggtgcccgtttcgctaacagcgttctaagcggtttcgaaggtgaaagagacgtcattgagccaactttcgtcgactccccattgttcaaggacgaaggtatcgaattcttcgcttccccagtcactttgggcccagaaggtgtcgaaaagatccacggtttgggtgtcttgtccgacaaggaagaacaaatgttggccacttgtaaggaaaccttgaagaagaacatcgaaaagggtcaaaactttgtcaagcaaaactaa

SEQ ID NO: 20 - protein, 338 aa, Kluyveromyces marxianus
MLRALTRRQFSSTAFNPYKVTVLGAGGGIGQPLSLLLKLNHKVTDLRLYDLKGAKGVAADLSHIPTNSTVTGYTPESKDSQEELAAALKDTEVVLIPAGVPRKPGMTRDDLFAINAGIVRDLATSIAKNAPNAAILVISNPVNSTVPIVAEVLKQNGVYNPKKLFGVTTLDVIRASRFISEVRGTDPTTEHVTVVGGHSGITILPLVSQTKHKSVIKGEELDNLIHRIQFGGDEVVQAKNGAGSATLSMAQAGARFANSVLSGFEGERDVIEPTFVDSPLFKDEGIEFFASPVTLGPEGVEKIHGLGVLSDKEEQMLATCKETLKKNIEKGQNFVKQN

SEQ ID NO: 21 - DNA, 1017 bp, Kluyveromyces marxianus
  CDS (1)..(1017)
atggttagcgttgcagtattaggatcatccggaggcattggccaaccactctcactcttgttgaagctggaccctcgcgtgtccagcttgagattgtacgacttgaagatgtcccacgggatcgccaccgatttgtcgcacatggactccaactccatctgcgagggcttcaacaccgacgagatcgcgctcgcgctcaagggcgcccagatcgtcgtcatccccgcgggtgtcccaagaaagcccgggatgtcacgtgacgaccttttcaagatcaacgccaagatcatcaagtcgttggcgttgcaaatagccgagcacgcgcccgaggcgcgcgtcctcgtgatctcgaacccggtcaactccttggtgcccattgtgtacgagactttgaagagcgtcggcaagttcgagccgggtaaagtgatgggaattaccacattggacattatccgctcacacacgttcctggtggacgtcttgggccgcaaggcgtacagcgtcgagaagttgcgcagcgcggttactgtggtgggcggccactcgggcgagaccattgttccgattttcaccgaccagaagttctacaggcgtctcagagacagagagctctatgacgcgtacgtgcatagggtccaattcggcggagacgaggtcgtgaaggccaaggacggcagcggtagtgctactttgtctatggcctgggcgggttacagttttgtgaagcagttgctcaacagcttgcacctagaaacaggcgaagacgtgcatccgatcccaacgtttgtgtacttgccgggtttaccgggcgggaaggagctccagcagaagttgggcacctctgttgagttttttgccgcgcccgtgaagctttccaagggtattgtggttgaagttgagcacgactgggtcgacaagttgaacgatgccgagaagaagttgattgcaaagtgtcttccaatccttgacaagaacatcaagaagggtctcgccttttcgcagcagacaaagttgtga

SEQ ID NO: 22 - protein, 338 aa, Kluyveromyces marxianus
MVSVAVLGSSGGIGQPLSLLLKLDPRVSSLRLYDLKMSHGIATDLSHMDSNSICEGFNTDEIALALKGAQIVVIPAGVPRKPGMSRDDLFKINAKIIKSLALQIAEHAPEARVLVISNPVNSLVPIVYETLKSVGKFEPGKVMGITTLDIIRSHTFLVDVLGRKAYSVEKLRSAVTVVGGHSGETIVPIFTDQKFYRRLRDRELYDAYVHRVQFGGDEVVKAKDGSGSATLSMAWAGYSFVKQLLNSLHLETGEDVHPIPTFVYLPGLPGGKELQQKLGTSVEFFAAPVKLSKGIVVEVEHDWVDKLNDAEKKLIAKCLPILDKNIKKGLAFSQQTKL

SEQ ID NO: 23 - DNA, 1125 bp, Kluyveromyces marxianus
  CDS (1)..(1125)
atgccagcagtatcatatgatgtccagcaacgggatatcctcaagatcgcagttctaggggcggcaggcggtattggccaatccttgtcgctcttgttgaagtcgaacgcttcttttttgttaccacgtgactcgtcaagacacataagcctagcgctatacgacgtgaacaaagatgccatcgtgggcacagcagcagacttgtcacacatagacacccctatcaccaccactccacactacccaaatgatgggaatggcggtatcgcacggtgcttgcaagatgcagacatggtcatcatcccagcaggtgtgcccagaaaacccggtatgtcacgtgatgacctaatcggtgtcaacgccaagatcatcaagtcgctaggaaacgacatcgcagagtactgtgacttgtctaaagtgcatgtattggttatttcgaacccagtgaactcgttggtcccactgatggtgtcgactttggcaaacagcccacacagtgcgaacacaaacatcgaggctagagtgtacgggatcacccatttggacctagtgagagcttccacctttgtgcaacagctaaactctttcaaatcaaataacgcacctgacattccggtcattggtggtcattccggagataccatcatccccgttttttccgtcttgaatcaccgcgcttctaactccggatacgctaatttgctagataatggcgttaggcaaaagttggtccacagagttcaatatggtggggacgaaatcgtccaagcaaagaacggtaacgggagcgcgacattatccatggcatacgcgggcttcaaaatcgcagcacaattcatcgaccttttggtcggaaatatccgcactatcgaaaatatttgcatgtatgttccgctcactaacaggtataataccgagatcgccccaggctctgacgaattaagatcaaagtacatcaacggaaccctttatttctcgattccactttccatcggaataaacggtatcgaaagagtccactacgagatcatggaacatctagacagctacgagcgtgagacgctactaccgatctgcttggaaactctaaagggtaatattgacaagggtctaagcttggtataa

SEQ ID NO: 24 - protein, 374 aa, Kluyveromyces marxianus
MPAVSYDVQQRDILKIAVLGAAGGIGQSLSLLLKSNASFLLPRDSSRHISLALYDVNKDAIVGTAADLSHIDTPITTTPHYPNDGNGGIARCLQDADMVIIPAGVPRKPGMSRDDLIGVNAKIIKSLGNDIAEYCDLSKVHVLVISNPVNSLVPLMVSTLANSPHSANTNIEARVYGITHLDLVRASTFVQQLNSFKSNNAPDIPVIGGHSGDTIIPVFSVLNHRASNSGYANLLDNGVRQKLVHRVQYGGDEIVQAKNGNGSATLSMAYAGFKIAAQFIDLLVGNIRTIENICMYVPLTNRYNTEIAPGSDELRSKYINGTLYFSIPLSIGINGIERVHYEIMEHLDSYERETLLPICLETLKGNIDKGLSLV

SEQ ID NO: 25 - DNA, 1518 bp, Issatchenkia orientalis
  CDS (1)..(1518)
atgggtgtccagtttatcgaaaataccattatcgttgtctttggtgcgtctggagatttagccaagaagaagactttccccgccctgtttggactattcagggagggccagctctcagaaacaaccaaaatcattgggtttgctcgatcaaaactatcaaatgatgacttgaggaacagaataaagccgtacttgaaattgaacaagagaacagatgctgaaaggcagtctctggagaagtttctgcagattctcgagtatcaccagtcaaactacgacgacagtgaaggttttgaaaaattggagaagctaatcaataagtacgacgatgaggcaaacgtgaaagagtctcacaggttgtactatttggctttaccaccgtctgtctttacaaccgttgcaacaatgttgaaaaaacattgtcatccaggtgattctggtattgctaggctaattgtcgagaaaccctttggccatgacttgagctcgtcccgtgagctacaaaagtctttagctccactttggaatgaagatgaattgtttagaattgatcattatttgggcaaagaaatggttaagaatttaattcctttgaggttttcaaatacgtttttgagcagttcttggaacaatcaatttattgacaccatccaaatcacttttaaggagaactttggaactgaaggacgtggtggttactttgattccattggtataataagagatgttatccaaaatcatttgttacaagtcttgactattgttttgatggaaaaaccagcggattttaatggagaatctatcagagatgaaaaggttaaagtgttaaaggcaattgaacaaattgatttcaataatgtgttggtaggtcaatatgataaatctgaagatggtagtaaacctggttacttggatgatgataccgtcaatccagattctaaagctgtcacttatgctgccttagttttaaatgtggcaaacgaaagatggaataatgttccgatcattctaaaggcaggcaaggccttgaatcaatccaaggtggaaattagaatccagttcaaaccagtagaaaatggaatcttcaaaaactctgctaggaatgagttggttattaggatccaaccaaacgaggcaatgtatttgaaaatgaacatcaaagtacctggtgtttccaatcaagtgtcgatttcagaaatggatttgacttacaagaataggtattcctccgaattttacattccagaagcttatgaatctttgattaaagatgccttaatggatgatcattcaaattttgttagagacgatgaattggacatttcatgggctttgttcactccattactagaacatatcgaaggccccgatggtccaactccaaccaagtatccttacggttccagaggtccaaaggagattgacgaatttttgagaaaccatggttatgtaaaggaaccaagagaaaattaccaatggccattaactactcctaaagaattgaacagttcaaagttt

SEQ ID NO: 26 - protein, 506 aa, Issatchenkia orientalis
MGVQFIENTIIVVFGASGDLAKKKTFPALFGLFREGQLSETTKIIGFARSKLSNDDLRNRIKPYLKLNKRTDAERQSLEKFLQILEYHQSNYDDSEGFEKLEKLINKYDDEANVKESHRLYYLALPPSVFTTVATMLKKHCHPGDSGIARLIVEKPFGHDLSSSRELQKSLAPLWNEDELFRIDHYLGKEMVKNLIPLRFSNTFLSSSWNNQFIDTIQITFKENFGTEGRGGYFDSIGIIRDVIQNHLLQVLTIVLMEKPADFNGESIRDEKVKVLKAIEQIDFNNVLVGQYDKSEDGSKPGYLDDDTVNPDSKAVTYAALVLNVANERWNNVPIILKAGKALNQSKVEIRIQFKPVENGIFKNSARNELVIRIQPNEAMYLKMNIKVPGVSNQVSISEMDLTYKNRYSSEFYIPEAYESLIKDALMDDHSNFVRDDELDISWALFTPLLEHIEGPDGPTPTKYPYGSRGPKEIDEFLRNHGYVKEPRENYQWPLTTPKELNSSKF

SEQ ID NO: 27 - DNA, 759 bp, Issatchenkia orientalis
  CDS (1)..(759)
atggtcaaggtttattcatattccaacaatgatgagatagctcaatcattggcaaagtttatagtttcacaacaggattcggttcttgcaaccaaggagaaattcaacattgctattagcggtggttcactaataggtattttagggaaaggcttactgaacaacaaagatatcaaatgggataagtgggtaatctatttcagtgatgagagaattgtaccattgagtgacaacgattccaattttggtgcttttgaaaaggaggttttggagaaactcgcccatgcagggaaagttgggccaacggttgttacaatcaacgaaaatttaattcatccggatgatcacactacggatggtgagattgcccagacatatgcatctgagttgcctgaatccggactggatttggtcttgctagggtgtggaccagacggccacacatgttctttgttcccaggccacaagttgttggaggagaacgagcttgatgttgctgcgcttcatgattctccaaagccaccaccaaggagaatcacattgacattcaagtatcttgctaaatgtactacactagcttttgttgccactggtgcaagcaagcaggaagctctaaaagagatttttggaaacgaaaacagccaactaccctgtgctattactaacaggttggtttcaaaagttaaggggggtatctgttggtttgtagatgatgatgccattgaaggcgtcgacgtccaaaccctcaagtactgattc

SEQ ID NO: 28 - protein, 251 aa, Issatchenkia orientalis
MVKVYSYSNNDEIAQSLAKFIVSQQDSVLATKEKFNIAISGGSLIGILGKGLLNNKDIKWDKWVIYFSDERIVPLSDNDSNFGAFEKEVLEKLAHAGKVGPTVVTINENLIHPDDHTTDGEIAQTYASELPESGLDLVLLGCGPDGHTCSLFPGHKLLEENELDVAALHDSPKPPPRRITLTFKYLAKCTTLAFVATGASKQEALKEIFGNENSQLPCAITNRLVSKVKGGICWFVDDDAIEGVDVQTLKY

SEQ ID NO: 29 - DNA, 1431 bp, Issatchenkia orientalis
  CDS (1)..(1431)
atgggtcaaaacttgattcttaatgcagcagatcatggttttactgttgttgcatacaacagaactgtctctaaagttgaccatttccttcaaaatgaagcaaagggtaaatccattattggtgcacactccattgaagaattatgtgctaagttgaagaaaccaagaagaattatgttgttagtcaaggcaggtaatccagttgatcaattcattgaacaattgttacctcatttagatgaaggcgatatcattattgacggtggtaactctcacttccctgactccaacagaagatacgaggaattaaagaagaagggtattctctttgtcggttctggtgtttctggtggtgaagaaggtgcaagatatggtccttctttgatgcctggtggtgcaaaggaagcatggcctcatattaaggacatcttccaatctatctctgcaaaggccgatggtgagccatgttgtgattgggttggtgatgcaggtgcaggtcattacgttaagatggtccacaatggtatcgagtatggtgatatgcagttgatctgtgaagcttacgatttgatgaagagagttggtggtttaactgacaaggaaatatctgatgttttcggtgaatggaacgagggtgttctcgattctttcttagttgaaattaccagagatatcttagctttcaacgataaggatggtaccccattagttgaaaagatcttagatactgccggacagaagggtactggtaaatggactgcaataaatgctttagacttgggtatgccagtcactttaattggtgaagctgtttttgcgagatgtttatccgctttgaagccagaaagagagagagcttctgaaatcttaaacggtccggaagttgaacaagtttctgctgaaggtagagcacaatttattgcagatttgatgcaagctttatatgcatcaaagattatttcttacgcacaaggtttcatgttaatcagagaagcagcaaaggaatacaactggaaattaaacttcccttctattgcacttatgtggagaggtggttgtattatcaggtctgttttcttggctgaaattactgcagcttatagggaaaaccctgacttagagaacttactattcaacaagttcttccaagatgctattcataaggcacagtctggttggagaaagactgttgcattagctgttacccaaggtattccaactccagcattctctactgcattgtctttctacgatggttacagatccaagaagttaccagctaacttgttgcaagcacaaagagattacttcggtgctcacactttccaaattttacctgaatgtgcagatgacgaaaagaaggttggtgattacatccatgtcaactggactggtaagggtggtaatgtttctgctagtacttacgatgct

SEQ ID NO: 30 - protein, 477 aa, Issatchenkia orientalis
MGQNLILNAADHGFTVVAYNRTVSKVDHFLQNEAKGKSIIGAHSIEELCAKLKKPRRIMLLVKAGNPVDQFIEQLLPHLDEGDIIIDGGNSHFPDSNRRYEELKKKGILFVGSGVSGGEEGARYGPSLMPGGAKEAWPHIKDIFQSISAKADGEPCCDWVGDAGAGHYVKMVHNGIEYGDMQLICEAYDLMKRVGGLTDKEISDVFGEWNEGVLDSFLVEITRDILAFNDKDGTPLVEKILDTAGQKGTGKWTAINALDLGMPVTLIGEAVFARCLSALKPERERASEILNGPEVEQVSAEGRAQFIADLMQALYASKIISYAQGFMLIREAAKEYNWKLNFPSIALMWRGGCIIRSVFLAEITAAYRENPDLENLLFNKFFQDAIHKAQSGWRKTVALAVTQGIPTPAFSTALSFYDGYRSKKLPANLLQAQRDYFGAHTFQILPECADDEKKVGDYIHVNWTGKGGNVSASTYDA

SEQ ID NO: 31 - DNA, 3761 bp, Issatchenkia orientalis
  CDS (1037)..(2764)
aaagatacaggtggctcagctgttgcaggtgtacttctagtatatggaattgacggctcatcaacattgcttaatcggcggcgctttaaagggttgttagtagtgtctatagtgtctatagtgtctatagtatctgtattgttgttgttgtttttgggattattggtgttaagactctcagtaagtttcggacgcagatctgtgttgaagagacgtacaagatccgcctttctgtcccttggcgacgccctgatcccatggcttgccaacagcgggcgaagatccttcacccgcagcgtccatggattgaactcgggcgataggtactgtgacgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaatatgactgacaaaatctccctaggtacttatctgtttgaaaagttaaaggaagcaggctcttattccatctttggtgttcctggtgatttcaatttggcattgttggaccacgtcaaggaagttgaaggcattagatgggtcggtaacgctaacgagttgaatgccggctacgaagctgatggttatgcaagaatcaatggatttgcatccctaatcaccacctttggtgtcggtgaattgtctgccgtcaatgccattgcaggttcttatgctgaacacgtcccattgatccatattgttggtatgccttccttgtctgctatgaagaacaacttgttgttacaccataccttgggtgacacaagattcgacaacttcaccgaaatgtcaaagaaaatcagtgcaaaggttgaaattgtttacgatttggaatcagctccaaaattaattaataacttgattgaaaccgcttatcacacaaagagaccagtctacttgggacttccttccaactttgctgatgaattggttccagcggcattagttaaggaaaacaagttacatttagaagaacctctaaacaaccccgttgctgaagaagaattcattcataacgttgttgaaatggtcaagaaggcagaaaaaccaatcattctcgttgacgcttgtgctgcaagacataacatttctaaggaagtgagagagttggctaaattgactaaattccctgtcttcaccaccccaatgggtaaatctactgttgatgaagatgatgaagaattctttggcttatacttgggttctctatctgctccagatgttaaggacattgttggcccaaccgattgtatcttatccttaggtggtttaccttctgatttcaacaccggttccttctcatatggttacaccactaagaatgtcgttgaattccattccaactactgtaaattcaaatctgcaacttatgaaaacttgatgatgaagggcgcagtccaaagattgatcagcgaattgaagaatattaagtattccaatgtctcaactttatctccaccaaaatctaaatttgcttacgaatctgcaaaggttgctccagaaggtatcatcactcaagattacctgtggaagagattatcttacttcttaaagccaagagatatcattgtcactgaaactggtacttcctcctttggtgtcttggctacccacttaccaagagattcaaagtctatctcccaagtcttatggggttccattggtttctccttaccagctgcagttggtgctgcatttgctgctgaagatgcacacaaacaaactggcgaacaagaaagaagaactgttttgtttattggtgatggttctttacaattgactgtccaatcaatctcagatgctgcaagatggaacatcaagccatacatcttcatcttaaacaacagaggttacactatcgaaaagttgatccacggtcgtcatgaggactacaaccaaattcaaccatgggatcaccaattgttattgaagctctttgctgacaagacccaatatgaaaaccatgttgttaaatccgctaaggacttggacgctttgatgaaggatgaagcattcaacaaggaagataagattagagtcattgaattattcttggatgaattcgatgctccagaaatcttggttgctcaagctaaattatctgatgaaatcaactctaaagccgcttaatgacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccgagcttggataggtcactctctattttcgtttctcccttccctgatagaagggtgatatgtaattaagaataatatataattttataataaaaactaaaacaatccatcaatctcaccatcttcgttgacttcaacattcataaatccggcataagttgatagacctggaattgtcatgatctttgcagctagtgcatataaatatcctgctcctgcacttattctaacttctctgattgggaagatgaaatcctttggaacacctttcaatgttggatcatgggagagagaatattgcgtctttgcaatacaaacaggcagcttgccaaaaccttgactctcatactcttcaatctgtttcttggccaactcagatagttcaatatcttttgctccatacatcttggtggcaattgttcttagtctatcttctaacgaaccatctaatgaataaagatagttaggaggttgactttcttccaacggtctttcctcggtagcacgtacaattgcctcagctaacttgattgcacccttaccaccttgtgaccaatggtctgactcaacagcatcaaatgcaccagccttgatggcttcttctctaatggtctcaatttccttctcggtgtcggtagcaaatttgttaattgcaacaacaactggggccccataactcttggcattttcaatttgttttcttaagttggatgatgcaccattcctaacaaattcaatgttttcagtgacgtattctgttggcaatacttgaccaggtttgacgtcgcttgcaccaccatgcaacttc

SEQ ID NO: 32 - protein, 575 aa, Issatchenkia orientalis
MTDKISLGTYLFEKLKEAGSYSIFGVPGDFNLALLDHVKEVEGIRWVGNANELNAGYEADGYARINGFASLITTFGVGELSAVNAIAGSYAEHVPLIHIVGMPSLSAMKNNLLLHHTLGDTRFDNFTEMSKKISAKVEIVYDLESAPKLINNLIETAYHTKRPVYLGLPSNFADELVPAALVKENKLHLEEPLNNPVAEEEFIHNVVEMVKKAEKPIILVDACAARHNISKEVRELAKLTKFPVFTTPMGKSTVDEDDEEFFGLYLGSLSAPDVKDIVGPTDCILSLGGLPSDFNTGSFSYGYTTKNVVEFHSNYCKFKSATYENLMMKGAVQRLISELKNIKYSNVSTLSPPKSKFAYESAKVAPEGIITQDYLWKRLSYFLKPRDIIVTETGTSSFGVLATHLPRDSKSISQVLWGSIGFSLPAAVGAAFAAEDAHKQTGEQERRTVLFIGDGSLQLTVQSISDAARWNIKPYIFILNNRGYTIEKLIHGRHEDYNQIQPWDHQLLLKLFADKTQYENHVVKSAKDLDALMKDEAFNKEDKIRVIELFLDEFDAPEILVAQAKLSDEINSKAA

SEQ ID NO: 33 - DNA, 3179 bp, Issatchenkia orientalis
  CDS (1052)..(2182)
gatttggacctacaaggtgctgtaaagagtatgaacacttctggggaggaggaatggaacagtgatgacgatgatgatgaagaaagtgacgaaagtaacgaaagtgattactattcttacgatgaaggcgaagaaacagatgatagtgagggagcccaagagggagaggaagacgaaaatgaacgaatcattgaagctctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatatgtttgcatcaaccttcagaagtcaagctgtaagagctgcaagatttactagattccaatccacttttgccattcctgagaagcaaatgggtgttatctttgaaactcatggtggtcctttacaatacaaggaaattccagttccaaaaccaaaaccaactgaaattttaatcaatgttaaatactctggtgtctgccataccgatttacacgcatggaaaggtgactggccattaccagcaaagttacccctagttggtggtcacgaaggtgcgggcattgttgttgcgaaaggttctgcagttaccaactttgagattggcgattatgctggtattaagtggttaaacggttcatgtatgtcatgtgaattctgtgaacaaggtgatgaatctaactgtgaacatgccgatttgagtggttatactcatgatggttctttccaacaatatgccactgctgacgctattcaagctgcaaagatcccaaagggtaccgacttatctgaagttgcgccaattttatgtgctggtgttactgtctataaagctttgaaaactgctgatttaagagcaggtcaatgggttgcgatttctggtgccgctggtggtctaggttctcttgctgtccaatatgcaaaggcaatgggtctaagagttttaggtatcgatggtggtgaaggtaaaaaggaactttttgaacaatgtggtggtgatgtgtttatcgatttcaccagatacccaagagatgcacctgaaaagatggttgctgatattaaggctgcaactaacggtttgggtccacacggtgttatcaatgtctctgtctccccagctgctatctctcaatcatgtgactatgttagagcaactggtaaggttgtccttgtcggtatgccatctggtgctgtctgtaagtctgatgtcttcactcatgttgttaaatccttacaaattaaaggttcttatgttggtaacagagcagataccagagaagctttggaattctttaatgaaggtaaggtcagatctccaatcaaggttgtcccattatctactttacctgaaatttacgaattgatggagcaaggtaagattttaggtagatacgttgttgatacttctaaataatgaagatgaagaaaacagcaaactttttatgactacccccaaccatctaacgatttatgatctatatatagctttctagaacatccatttatttattcacttactcatgtatttatattatataatacaaaataactaattacaatgtgtacatttttttttttcattaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttc

SEQ ID NO: 34 - protein, 376 aa, Issatchenkia orientalis
MFASTFRSQAVRAARFTRFQSTFAIPEKQMGVIFETHGGPLQYKEIPVPKPKPTEILINVKYSGVCHTDLHAWKGDWPLPAKLPLVGGHEGAGIVVAKGSAVTNFEIGDYAGIKWLNGSCMSCEFCEQGDESNCEHADLSGYTHDGSFQQYATADAIQAAKIPKGTDLSEVAPILCAGVTVYKALKTADLRAGQWVAISGAAGGLGSLAVQYAKAMGLRVLGIDGGEGKKELFEQCGGDVFIDFTRYPRDAPEKMVADIKAATNGLGPHGVINVSVSPAAISQSCDYVRATGKVVLVGMPSGAVCKSDVFTHVVKSLQIKGSYVGNRADTREALEFFNEGKVRSPIKVVPLSTLPEIYELMEQGKILGRYVVDTSK

SEQ ID NO: 35 - DNA, 3181 bp, Issatchenkia orientalis
  CDS (1015)..(2181)
tttttcttttttttttctttttttttcctctttttcttttgttttgtttcgtttcttttccgccagttcccgttttccatttccggaacaacaatgggactccactgttttcttccccccttcccttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggcccagccgaaaaaagaaatggaaaacttggccgaaaagggaaacaaacaaaaaggtgatgtaaaattagcggaaaggggaattggcaaattgagggagaaaaaaaaaaaggcagaaaaggaggcggaaagtcagtacgttttgaaggcgtcattggttttcccttttgcagagtgtttcatttcttttgttttatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttattttatgtttctaatctcaaagagtttacactttacaaggagtttctcaaatactatcaagacattgaatagatcaaacattaaaatggtgtcccctgctgaaagattatctactattgcgtccacaatcaagccaaacagaaaagattctacatcattacaaccagaagactatccggaacatccgttcaaggtgacggttgttggttccggtaactgggggtgtacaattgccaaggttatagcggaaaacaccgttgagagacctcgtcaatttcaaagagatgttaatatgtgggtctatgaagaattgattgaaggcgaaaagttgactgaaatcataaataccaaacacgaaaacgtcaagtacttgccaggtatcaagttgccagttaacgttgttgcagttccagacattgttgaggcttgtgcaggctcagacttgattgtctttaatattcctcaccaatttttaccaagaattttatcccaattaaagggtaaggtgaatccaaaggctagagcaatttcttgtttgaaaggtttggatgtcaatcctaatggatgtaagttgctctccactgttattactgaagagttgggtatttattgtggtgccttatcaggtgctaatttagctcctgaagttgcacaatgtaaatggtcggaaacaactgttgcatatacaattccggacgatttcagaggtaaaggcaaggatattgaccatcaaattctaaagagtttgttccatagaccttatttccatgttcgtgttattagtgatgttgcaggtatttccattgccggtgcactcaagaatgtcgttgctatggctgctggatttgtcgaaggtttaggttggggtgataatgcaaaggctgcagtcatgagaataggtttggtggaaaccattcaatttgccaagacttttttcgatggctgtcatgctgcaacctttactcatgaatctgcaggtgttgccgacctaatcactacctgtgccggcggccgtaacgttagagttggtagatatatggcacaacattctgtctctgcaacggaggctgaagaaaagttgttgaatggccaatcctgtcaaggtatccacacaactagggaagtttacgagttcctctccaacatgggcaggacagatgagttcccactatttaccaccacctaccgtatcatctacgaaaacttcccaattgagaagctgccagaatgccttgaacctgtggaagattaaataacctcagggagaactttggcattgtactctccattgacgagtccgctaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaatagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacatcactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccgagctcgacattgcaagcaagatgtcctgggaacagtttctaaccatgagaaagcaacagagaagattgggaggcattgggtccattgcagcagcaattttggcaatgggcgcctccttctcgtatttctcacagattgaaatcgatcctac

SEQ ID NO: 36 - protein, 388 aa, Issatchenkia orientalis
MVSPAERLSTIASTIKPNRKDSTSLQPEDYPEHPFKVTVVGSGNWGCTIAKVIAENTVERPRQFQRDVNMWVYEELIEGEKLTEIINTKHENVKYLPGIKLPVNVVAVPDIVEACAGSDLIVFNIPHQFLPRILSQLKGKVNPKARAISCLKGLDVNPNGCKLLSTVITEELGIYCGALSGANLAPEVAQCKWSETTVAYTIPDDFRGKGKDIDHQILKSLFHRPYFHVRVISDVAGISIAGALKNVVAMAAGFVEGLGWGDNAKAAVMRIGLVETIQFAKTFFDGCHAATFTHESAGVADLITTCAGGRNVRVGRYMAQHSVSATEAEEKLLNGQSCQGIHTTREVYEFLSNMGRTDEFPLFTTTYRIIYENFPIEKLPECLEPVED

SEQ ID NO: 37 - DNA, 2587 bp, Issatchenkia orientalis
  CDS (433)..(2115)
ccagttcggaaactaagacataacaaaaaagcatgggatgaatggggaaaaaatttagaaatgtaaaaaaaaataggaaaggggcttttcctattgtcaataaaacaactctgttaggagaaaatagttttataatattttcatggactctttcccccacatttattaccgaattgggaaattcccgggcaatatccctcgctccttagaaattccgtactcccggggagggctaactctccgagttattcctaacactctggggggaacaacattactcatagaatactataaataccgagcgtaccgcatatatgtagtatgtgacagaaattggtatccgataatcccttaccttggcaaggttttgtttcaataaaacattcaaataacttaaattcaaataatattcatttaaattgctttaaaaaaatggctccaactgctgttgatatccataacgagtacaaacagaatgtttccaacgaacaggaaattcctttcaacaaaactgaaagaaagtcatcgattgcatctaaattaggactgaatccagacgctaagattcactacaattctgctgttcctatattatacgaagatggtttaaaggaaaaaggtacaaccatttcctcttctggtgcattgattgcattctctggttccaaaacaggtagatctccaaaggacaaaagaattgtcgatgaagagacttcaacagacaacatctggtggggtccagtcaataagaaggttgatgaaaacacttggaatatctcgaaatctagagcgattgattatttgagaacaagagagaaggtttacattatcgatgcttttgctggttgggatccaagatacagaattaaggttagaattgtctgtgctagagcttaccatgctttgttcatgaagaatatgttaattagaccaacaacggaagaattaaagaactttggtgagcctgatttcaccatttggaatgcaggtcaattccctgctaatgtttacactaagggtatgacttcttcaacttctgttgaaataaatttcaagtctatggaaatggttatcctaggtactgaatacgcaggtgaaatgaagaaaggtatctttaccgttatgttctacttgatgccaatcagacacaaggttttaactttacactcttctgcaaatcaaggtaaaaaggatggtgatgtcacattattctttggtttatctggtacaggtaaaacaaccttgtctgcagatcctcatagagaattgattggtgatgatgaacattgctggtctgatcatggtgttttcaacattgaaggtggatgttatgctaagtgtttggacttatctgctgaaagagaacctgagattttcaatgcaattaggtttggatctgtcttggagaatgttgtctatgatccagttgatagaactgttgactattccgctgctaatgtcactgaaaatactagatgtgcttatcctatcgactttattccttctgctaagatcccatgtctggcagattctcatccaaagaatattgttcttttaacttgtgatgcaagaggtgttttgccacctgtctccaagctaactaatgcacaagtcatgtatcactttatctctggttacacctccaagatggcaggtaccgaagttggtgtcactgaaccagaagcaaccttctctgcatgttttggtcaacctttcttagttttacatccaatgaaatacgcacaacaactctctgataaaatggctgaacattcttccaccgcttggttattgaataccggttggactggtcaatcttatgttaaaggtggtaagagatgtccattgaagtatactagagcaattttagatgctattcactctggtgagcttgcaaaacaggaattcgaaacataccctactttcggtttacaagttccaaaaacttgtccaggtgtcccagaaagtgttctgaacccatctaaacactgggctactggtgaagctgatttcaaggctgaagtcactaacttggctaaattatttgctgagaactttgaaaagtattctgcagaatgtactgcagaagttgttgctgctggtcctgctttataaatggtttgatatcaatattatgactcggttatgtttgtgtatttaattccctctttttgtttattctttttttcttccacccttgtttatcattagtgtcgcccttttcttttcaaaggacaattattagcatttatataaaattcaattcagtactatattcgatccttttaatcacttcagtgtgtcgtgtaggattaataccagagctagaaacaatatggattggacatgtctaccaattatattatatgtatatatgaatctaattatctctatataaaatcaattaactaaatgtttattgatcggtaatttcccatcaattaatactggtttaggaatatatttgtcatcaacttgaatctcgaaacctggtttacaggatagtttttcatgaaattggataatcagtttcgatagtggttccgcgtaccatctctcattgttccatttgaaaaagccaacatgg

SEQ ID NO: 38 - protein, 560 aa, Issatchenkia orientalis
MAPTAVDIHNEYKQNVSNEQEIPFNKTERKSSIASKLGLNPDAKIHYNSAVPILYEDGLKEKGTTISSSGALIAFSGSKTGRSPKDKRIVDEETSTDNIWWGPVNKKVDENTWNISKSRAIDYLRTREKVYIIDAFAGWDPRYRIKVRIVCARAYHALFMKNMLIRPTTEELKNFGEPDFTIWNAGQFPANVYTKGMTSSTSVEINFKSMEMVILGTEYAGEMKKGIFTVMFYLMPIRHKVLTLHSSANQGKKDGDVTLFFGLSGTGKTTLSADPHRELIGDDEHCWSDHGVFNIEGGCYAKCLDLSAEREPEIFNAIRFGSVLENVVYDPVDRTVDYSAANVTENTRCAYPIDFIPSAKIPCLADSHPKNIVLLTCDARGVLPPVSKLTNAQVMYHFISGYTSKMAGTEVGVTEPEATFSACFGQPFLVLHPMKYAQQLSDKMAEHSSTAWLLNTGWTGQSYVKGGKRCPLKYTRAILDAIHSGELAKQEFETYPTFGLQVPKTCPGVPESVLNPSKHWATGEADFKAEVTNLAKLFAENFEKYSAECTAEVVAAGPAL

SEQ ID NO: 39 - DNA, 2562 bp, Issatchenkia orientalis
  CDS (363)..(2174)
ctactatgtacactgtataagtaaaaagacgatacccccctcccactctgggtgctacggtgtagatctctccgtaaacacaaaaaggcggctcagatgataattggggtccgggcgcaaccggaaggggggagagaggggagcgatggcttctcctccggggggctacgggagtttcctctttgggaaggataaagaggggatggattgatacaagattctgagaacctattacgatgatgttcagtggtattttgtcttttgttatttaaagggaggggactttcctcaataccttagttgtaaaattacgctattatctttaaccctttcttttgagcaataattaaaaagaacacgccatgacaaaagattgctgtgaggttacaaagattccagtgggagaggaggccaaagtgaccgttcctcgttccacaagattatcagcaactggtccagttgaatgtgatttatctggtttccaagttttgaactctccacttttcaacaagggcactgcattcactattgcagaaagagaagcatttggtttaaacgggttacttcctccggttgtaaacactctagaagaacaagttgagagaagctataagcaactacattttctcaagactccattggcaaagaatgacttttgcacgtcattgagattgcaaaacaaggttctgttttataggttagtcaaggaacacattaaggagttgattccaattgtgtatacaccaacagagggtgatgcaattatcgcttattctgacagattcagaaaaccagaggggttattccttgatattacaagaccaaatgaaattgatcaaagactggaacagtttggagaagataaagatgtggattacattgttataacagattctgaaggtattctaggtattggtgaccaaggtgttggcggtgtcagaatcacaattgcaaaggctgctctaatgactgtatgtgctggtttgcatccaggtagagttgtctcttgtgtcttggacgttggcactaacaatgtgaaattgctagaagatgatctatatcttggtaacagattcccaagagttagaggtaaggagtatgacgattttgtgaataaaactattcgtgcaatgaagaagagatttccaagtgctgttattcattttgaagattttggtgttacaactgctagacctgtgttggaaagattcagagatgaaattccttgctttaatgatgacatccaaggtaccggcgctgttgtcatggcttcgatggcggctgctcttaaattaacccatagaaatctactagactccaaagttttgatttatggggctggttcagccggtttgggtattgccgatcaaattgttaatcatatggttagccatggtgcgactaaagaagaagctagaaggaagatttactgtatggacaggtatggtttgattttaaaaggtatgacttcgaattctcctgctcaagaagattatgctcatgatccaaaagattgggaaaatatttcaaccacttcgttagtggatgttatcgaaaaagtcaagcctactactttagttgggtgctccacgcaagcgggcgctttcaatgaagaagtcatcaaaacaatgtataaacataatccaagaccaatgattttcccattgtccaacccaactagattacatgagtgtttccctgaagacgcacttaaatggaccgatttcaacgctttagttgccactggttctcctttcccacctgttgaaggtcatgttatttctgaaaataacaattgttttgccttcccgggtattggtctaggtgcagtgctcgctagaactactaggatatcagacaacatgatttcggctgccgttgacgagctagcttctctttctccagctcaaaaagatcctaaattgggccttttacctccaattgaggaaatcgacgagacctctgcaagaatcgcaactgcagttatcttgaaggctgtcgaggaaggatttgcaagagtagaagaagaagattctccattaggtggtaaagttaaaattccaagagagtttgatccatgtctaagatgggttaaagaacagatgtggcatccaatttacagaccaatgatcaaagtcgcacactcagacaatattcatactcaccaatactaatggacacatacacattatcaaatgcatttattcctaatatcacactaaaacgtattatataattttaatctttatagacttcatagcaccaattggatttgctttctttcagaataccgcacttaatctcaatgtacgtaacgtaggcaaaatctgtcgataaggatctgtatgccgtaaacggaaactccaagcgcccagaaaacttacattatattcttgccagtttcatctcaccagccagtcacagtttaaaaggtttgattgcgtttcttgtttcgtcggattcagtgctaattggtaacgcactgtaccgccacaccaaagcaaaaatgcagaaacaaacaacaatgagtgtatgtttaccaactttggttttgaaagaaaa

SEQ ID NO: 40 - protein, 603 aa, Issatchenkia orientalis
MTKDCCEVTKIPVGEEAKVTVPRSTRLSATGPVECDLSGFQVLNSPLFNKGTAFTIAEREAFGLNGLLPPVVNTLEEQVERSYKQLHFLKTPLAKNDFCTSLRLQNKVLFYRLVKEHIKELIPIVYTPTEGDAIIAYSDRFRKPEGLFLDITRPNEIDQRLEQFGEDKDVDYIVITDSEGILGIGDQGVGGVRITIAKAALMTVCAGLHPGRVVSCVLDVGTNNVKLLEDDLYLGNRFPRVRGKEYDDFVNKTIRAMKKRFPSAVIHFEDFGVTTARPVLERFRDEIPCFNDDIQGTGAVVMASMAAALKLTHRNLLDSKVLIYGAGSAGLGIADQIVNHMVSHGATKEEARRKIYCMDRYGLILKGMTSNSPAQEDYAHDPKDWENISTTSLVDVIEKVKPTTLVGCSTQAGAFNEEVIKTMYKHNPRPMIFPLSNPTRLHECFPEDALKWTDFNALVATGSPFPPVEGHVISENNNCFAFPGIGLGAVLARTTRISDNMISAAVDELASLSPAQKDPKLGLLPPIEEIDETSARIATAVILKAVEEGFARVEEEDSPLGGKVKIPREFDPCLRWVKEQMWHPIYRPMIKVAHSDNIHTHQY

SEQ ID NO: 41 - DNA, 3743 bp, Issatchenkia orientalis
  CDS (1010)..(2746)
atttgtgtgtgtgtgtcagtgtaagtgttagtatacttgttttcctctttcccctagagttggtggtgtgttttgttggaacgtacattagatgcataatgcgtgacaccgccatgatggttgtattctaccaatgagacatggccgctgatcctgttgtgtgggtcatgggacatcacctcttgggggggattctcctataattggcaccgtgtatgcctcaaccactaacttccaccctataactgaatatattacataagcaaatctactttttgtttgtgttgatcgccatcgttgaaattcgcgcaacttctggtggctcaacgctgctgttctatcggtatcctaagagatgtctttgccctgagtctagggtaaactatccaccttcgttgctgtttgactagacagctactaactttacggtagtaaatgaataacggctcgctctcatgatcacttctctacatcaccctaacaagtgtattatttttttttcaggtgggtgttgctgttggtgctagccttagtgccctcgttaatagttgaacaaacactggcatttggagtataatgaaaagggatcactaccccccgcttcctgttccgcttctcccttccggaaaaaccacccaccctttcttttcccccactaatgtatgaatttttccgttcccaggggaatggcccacttggttctctgttaacccacacaattttgacgcatcccacacaccttttttttttctaccccacactttcccttgaaaaatctccaatttgaactggcaattttcaccccccaccacttgcattcattagtgagtcaatccatcccgcggtcggagattcggaatccacctactggtaatctgtaatctatattcccgctgaccctttataaatgaactattgtcgtcaattgcggtagtgctccaacaaattgtaaggaccttctttaaccttttcgattcaatccatctccacataaacctagttgcacacaatgttactcagatcactaaactcttctgctcgttgtgtcaaacaaacaaccagaacaaaggttaggtatctcagccacgtcagtggtgcaagcatggcgaaacctacattgaagaacaactcgagagaatccaacaaatccagaaactatctaattgctgctgtgacagcattggctgtatcaacctcaattggagttgccgtacatgtgaaggaccccttgtataacgatgctaccggcagtgattctccgagaagtatatctgttgacgagtttgtcaagcataattcacaaaacgactgttggattgcaatcaatggcaaggtttatgatttcactgattttattccaaaccatccaggtggggtacctccattagttaatcatgctggttatgatggtactaaactttatgagaaattgcatccaaaaggtacaattgagaaattcttgccaaaggataagtttctgggtgtgttagatggtgaagcgccaaaattggaagcagactatttggtggacgatgatgaacaagagagactggattatttgaacaacttacctcctttgtcatctattcagaatgtttatgatttcgaatacttggccaagaagattttacctaaagatgcctgggcatattattcttgtggtgccgatgatgaaatcacaatgagagaaaaccattatgcttatcaaagagtttatttcagaccaagaatttgtgttgatgtcaaggaagttgatacttcttatgaaatgttaggcactaaaacctctgttcctttttatgtatctgccaccgctttggctaaattaggccatcctgatggtgaatgctcaattgctagaggcgctggtaaggaaggtgtcgttcaaatgatttcgaccctttcctcaatgtcattagatgaaattgccgctgctagaattccaggtgcaacccaatggttccaattatacattaatgaggatagaaatgtcgctaaaggtctggtcaaacatgcagaagacttgggtatgaaggctatctttataactgttgatgctccttctctaggtaacagagaaaaggataaaagattaaagtttgttaatgacaccgatgtcgatttgggtgattccgcagatcgaaacagtggtgcttcaaaggcactatcttcgttcattgatgcttctgtctcttggaatgacgtcaaagcggtcaagtcgtggactaaattgcctgtcttagttaaaggtgttcaaacagttgaagacgttattgaagcttacgatgctggttgtcaaggtgttgttttgtcaaaccacggtggtaggcaactagatactgctcctcctccaatcgaattattagctgaaactgttccaactttgaagagattgggtaaattaagaccagattttgaaattttaattgacggtggtgtcaaaagaggtaccgatattttgaaagcagtcgcaatcggtggccaagatgtcagagtttcagttggtatgggtagacctttcttatatgccaactcttgctatggtgaagcaggtgttagaaaattaattcaaaatctaaaggatgaattagaaatggatatgagattgttgggtgtcactaaaatggaccagctatcttcgaaacatgtcgatactaaacgtttgattggtagagatgcgatcaactatttgtatgataatgtatacagcccaatcgaaaccgttaaattcaacaatgaagattgattgttggaaatatattattcataaaggcgaaaacattcccttggtattttattccaaatttatgatacatagacgtattttttatatataaagttatattattaatgattcaagaaaaagttcaaataaactaatggatcaacctatttcgaccctttcttcattgctacttcttccttaagcaacagatgattaagtagatactgtttttttagccaatagtatctcgccgaggagttatacttgactagctcttgctcaagaatcttcctaagacgtactagcctagcatagtaatctgtttgtttctgtattgtttgttctaactgttctacagtcattgaatcaatatctccaatgtcttcgacgttgacaactttcccccccttggcagcattctcttttttgttggaatacgacattaaagattccttgattttctgggtaccttcaatgaccattgagggattaaatttgatttctttgattttataatggtcggctattagctcttccacttcgtcatcatgatcatcagatatgtcacgttgccttttcaatttattaaaattgtttatcagtttattgtgatcttgtatcaattcattgcgtactcttttctcaatatcaaaagctattttcttcccgctagactcaaaatcaactctgaagtcattttctcgctggaattcatgtatttcatggattaattctctattgatattctcatatgcatcctgtaaactgttgccgttgatattatgaaccgcctttaaatgtttcaataaggcatctgctctagtaaatgccttcagacattcaggtaataaacagtaaaatggcttctcggctgtatgcgtcctaatatgagacaatagcgcaaatctactgtgttgtggagtaccatatcttggacaatttccccacttacatgaatggtctgtgttgggttgtttaaaatgattagtatttatgtgattaaccatctcatccattt

SEQ ID NO: 42 - protein, 578 aa, Issatchenkia orientalis
MLLRSLNSSARCVKQTTRTKVRYLSHVSGASMAKPTLKNNSRESNKSRNYLIAAVTALAVSTSIGVAVHVKDPLYNDATGSDSPRSISVDEFVKHNSQNDCWIAINGKVYDFTDFIPNHPGGVPPLVNHAGYDGTKLYEKLHPKGTIEKFLPKDKFLGVLDGEAPKLEADYLVDDDEQERLDYLNNLPPLSSIQNVYDFEYLAKKILPKDAWAYYSCGADDEITMRENHYAYQRVYFRPRICVDVKEVDTSYEMLGTKTSVPFYVSATALAKLGHPDGECSIARGAGKEGVVQMISTLSSMSLDEIAAARIPGATQWFQLYINEDRNVAKGLVKHAEDLGMKAIFITVDAPSLGNREKDKRLKFVNDTDVDLGDSADRNSGASKALSSFIDASVSWNDVKAVKSWTKLPVLVKGVQTVEDVIEAYDAGCQGVVLSNHGGRQLDTAPPPIELLAETVPTLKRLGKLRPDFEILIDGGVKRGTDILKAVAIGGQDVRVSVGMGRPFLYANSCYGEAGVRKLIQNLKDELEMDMRLLGVTKMDQLSSKHVDTKRLIGRDAINYLYDNVYSPIETVKFNNED

SEQ ID NO: 43 - DNA, 3695 bp, Issatchenkia orientalis
  CDS (1001)..(2698)
ctcgtttcctgtgatatgtttgccgtgcgcctggtcgattttcaccttctttgaaatccgagttcgggaatgcaattgggaaaaagccaaggagaaagaaaacaaaaagagagttgcgtagaaactcggaatgctcgaagaaaccagacagttgatggctggtttccgttttgaggacgcttggtgtgtgtaacttggatttgcacactagagccgtctctgcattgtattaaggtgtaaggacggtgaatcatcgcgatggagcggggttttttcttttggcaggtttttccgcggaaggcgagagggcgagagggggggggggtgtatgtagttcatatttcggcattactacaaggatgtttccgtacattgcatggtactggggttctccttttcttgcacatctccataaactaaatatcaatagatgtatccgtttggaatctcatgacttttggtgtgtggtctgtgtcttcccagttatctacttgagtgattatgatccagttttcaccattggttacataccaaacagagaacttatacgcaccagaacgccttttgtgtctttttgtttctcaagtatttctatcagtttccttcatgtatcccgggactccattgtcctcggtagtgcctaccaatttaatgtttgactccttgcgttttctcctgtcgcggacaaacggtgcggctcccccgatgattcacgtaataagccggagtcaaccacagaggtcccctatgactcaacaaggcctcgtagaaactcggcttctcggagaaagagtcttttctttttcactggaaaatatttttttttcctttatattcttttgaaccaaaatgtggctactataaaagtgcctttattccccagcttttctagcatgattgagtcaccttccacaatgagtcttctttattgttagtattgtgaatattatccgtgcagttttcaagaacgtaaatcaacagcagtgataataccttcaaaatgttaagatcccagttcaaaaacattttgaaaaatgttaacaagaaccattctctaaggagaacttttacttccagcacctcaaaggctggaaaaaatgcttcatacaatgccaagattatatctgcaaccgtggcctcgattgttgcagcagctggctcttatatgttggtccagccttcactagctaatgatgaggcacagtctgctaatccaactaggaagatctctgttgacgaatttgttaaacacaaccatgccgatgattgttggatcactgttaacggtaacgtctatgacttgactgatttcatttcaatgcatccaggtggtactaccccattgattcaaaatgcaggtcacgacgcaactgaaatttacaacaagattcatccaaagggtacaatcgagaacttcttaccaaaggaaaagcaattgggtgttttggatggtgaagctcctaaaatcgaagttgtgcttgacgaaaaggagaaacacagattggagttgttgaatcatctccctgctctttccagaattcaaaacatttatgatttcgaacatattgcttctagagttttgagcgaccaagcatggaactactattcatgtggtgccgaagatgaaatcaccttgagggaaaatcattatgcttaccaaagaatctactttaagccaaaatgttgtgtcaatgttgcagaagttgatacctctcatgaaattttaggtacaaaagcttctgttcctttctacgtttccgcagccgcttctgcaaagttggggcacgaggatggtgaatgttccattgctagaggtgcaggtaaggaaggcgttattcaaatgatttcttccttctcttccaactctttggaggaaattgcagaatccagaattcctggtgcaacacaatggtttcaattatacgttaatgaagacaaggatgttgtgaagaagactttaaaaagggccgaaaacttgggtatgaaggccatctttgtcactgtggacgctgctagtagaggtaatagagaaaaagacattagaatgagaattaccgaagatacagatgagttaatcgacgattcttctgttagagctggttctacctctggtgcattgccagctttcattgacaagaggctgacttgggatgaagttaaggatatcatttcatggaccaagttaccagttttgctgaagggtgttcaaagaactgatgatattgagaaggcaattgatattggttgtaagggtgttgtcttgtccaatcatggtggtagacaattagatacttctcctcctccaatagaagttatggctgaatctgttccaatcctaaagcaaaagggtaaactggatccaaatttcagtattttcgttgatggtggtgttagaagaggtacagatattttgaaagctttggctattggtggcagagactgtaaagttgctgttggtctgggtagacctttcctttatgcaaatactggttatggtgaaaagggtgtcagaaaggccgtgcaaattctaagagaagaattaaaggctgatatgagaatgttgggcgttacctctttgaacgagctagacgactcttacattgacaccagaagattactaggtagagatgctgttaaccacatatacaacaacaactactacccaatgtctaagattcaattcaaaaacgaaaaataagtctgatatttgctaaattgaaatgaaccttaccatgccacatctatagacatcaaaacattttcaatttgtcgatatcttttgcatatcaaagtaataccaagcatgttcaaaaagaaaagaaagcataactttaatactctattcgaaacattccgatccacaacacattagtctttttaggcccgttgttcatctttctattactttattcctaactgtatttttataattccgggtttataaaagattaaactaatatagcgcattctttttgggtacaaacatacataacggagctcattcatacatcgcttttcagttcgactggtgtttcggatgcctctttttctaaggagctagattctggccccacactagtctttgaactcgttgctcccttaccacccttaccaccagccttacttgtaggtttttcagtagcatactctgcgtgtttgactaaattcccttccttaactttgtgccagcttggccatatcattaaatacccactgaaacttctaacaactcttcgaccttcctgatgggcctttgaaattgtatctaccaaacctgccttcaagggatgttctttatatcccctgacgtctttcatactttgaacttcctctgggacgtcttcctttccatatttttcccattggcccggcttgtttttagatttgtctatctcacggaaaattgaggggttcatacttaatccactcacaccaaccctgatgttagaagacagttttgctaaattatttacattctgacttgtgtttgtcgatataactgaatcagatggtttcatcgatgattctcgggagattggttctgatggcgtcaccggcgtctcagatgctgctggatttagctttagtttgagccgtttaataggttcttcatttaatgtttgttttctttttttgtcgtagaaatgtgctgtgagatcaggaaattgttcaagctgttcacgactcagttttag

SEQ ID NO: 44 - protein, 565 aa, Issatchenkia orientalis
MLRSQFKNILKNVNKNHSLRRTFTSSTSKAGKNASYNAKIISATVASIVAAAGSYMLVQPSLANDEAQSANPTRKISVDEFVKHNHADDCWITVNGNVYDLTDFISMHPGGTTPLIQNAGHDATEIYNKIHPKGTIENFLPKEKQLGVLDGEAPKIEVVLDEKEKHRLELLNHLPALSRIQNIYDFEHIASRVLSDQAWNYYSCGAEDEITLRENHYAYQRIYFKPKCCVNVAEVDTSHEILGTKASVPFYVSAAASAKLGHEDGECSIARGAGKEGVIQMISSFSSNSLEEIAESRIPGATQWFQLYVNEDKDVVKKTLKRAENLGMKAIFVTVDAASRGNREKDIRMRITEDTDELIDDSSVRAGSTSGALPAFIDKRLTWDEVKDIISWTKLPVLLKGVQRTDDIEKAIDIGCKGVVLSNHGGRQLDTSPPPIEVMAESVPILKQKGKLDPNFSIFVDGGVRRGTDILKALAIGGRDCKVAVGLGRPFLYANTGYGEKGVRKAVQILREELKADMRMLGVTSLNELDDSYIDTRRLLGRDAVNHIYNNNYYPMSKIQFKNEK

SEQ ID NO: 45 - DNA, 1960 bp, Issatchenkia orientalis
  CDS (411)..(1343)
gtacgactcatggggctttacaaacaccttacatgttgtatacagtagagcggggggcaacaacctatgggggtgaatatgaagctcttatgacgagtcaccccgatttccccgttatccgacacgagcgatttgaaagcccgttttcttacctgttagggaaattgtccgtgggcatgtttctccgttccaagggttccgtttgccgtcaggtatctaaggatagggcacaatagtcagcggggaaacggagttcccgagggtgtcggagatactccagctaattgacccctagataaatattcatgattaattcgactattataaggtcattcttcccatgttctttccattcatctatataaggtgtccctggttcctttattaagaagaaaacaactgcaacacttatgactttgaagtccaaatcaaaatccaaaaagaagcacgcatcctcaaaaccactggaatcatctaataaaatgtccaaaagtagtgttgaacaccatgaacatacctcaaataaggaaaatgaccatatttccttgcattcccgcttgacgaatattgaacatcagatcatgggcaaagtacatacaagtgatgacggcgcctatgttatcttagacaacaaaaagtatcctaagtcagaattattgaaggcctttggtggttttatgaaccctggttgggcagtgccttccgaacacaagtttggtaatccagctcctttgggtctatctgcatttgcgtattgtacttttgttgcatctttggtcaacatgcaaactagacatgttgaaaatgatgctgttaatgtcggtgctgcaatgttttatggtggttttatccagttcattgccggactttgggaaatatcgcttgaaaacgcttttggtggtttggcattttgctcttttggaggttactggatggcatcggcctcaaaccatatcccctggttccatattgctagctcttatactacagaagcagaattcaaatcaggtatgggatttttctaccttggttggctactctttacaataatcttgctagcttgttcaatcaaatctaccattttatttttcctgttgtttgtgctggtctttatgagattgctgttattaacatgttggaagtttgcggacagtcatgcctgtgagtttgctgctggtgttttcggtgttttggcatctctgttagcatggtatcatgcatatgcaggtattgcaacacctcagaattcttactatgttgttaatccaacacctatgcctgttattggatcaaagagcaaagatatgtttgattctgacgactttgaccaatcttcatcttgatctagagtccctctaattcttcctgttgtttttttctcggttcacttcttccaccactttgggtttgaaacctccttttataaactgtgtttttatccatttttttttgttcttactgcttacaacttaatataatacactatataacaaaaaattccgtttatttctacatcactatgtgacgaattgtatattcatctgttcatcaaggtcccacacttcttacctgaaatgttccaccaatattagaaaacctgcacttatcactctaacttatgtatagtacttagaggaagttacaattatcagtgtctgtaccggccacaatggttgatatcctccaatacgatacagccccacatacttaagttttcacatattgcgcataataaataggtgcaatctgacccttgccaaataattcggtcattccacgtctagaccggacaacaagtggaaaaaatagatctatgaggatctgagcaacgtcaatcaaatccatattattctcctgcccgattttgaccgttgtgagtcctacagaacacagatggacacacactgagtcatctccacaactacacccccatctcgtataaatgagtgctcgatcactg

SEQ ID NO: 46 - protein, 310 aa, Issatchenkia orientalis
MTLKSKSKSKKKHASSKPLESSNKMSKSSVEHHEHTSNKENDHISLHSRLTNIEHQIMGKVHTSDDGAYVILDNKKYPKSELLKAFGGFMNPGWAVPSEHKFGNPAPLGLSAFAYCTFVASLVNMQTRHVENDAVNVGAAMFYGGFIQFIAGLWEISLENAFGGLAFCSFGGYWMASASNHIPWFHIASSYTTEAEFKSGMGFFYLGWLLFTIILLACSIKSTILFFLLFVLVFMRLLLLTCWKFADSHACEFAAGVFGVLASLLAWYHAYAGIATPQNSYYVVNPTPMPVIGSKSKDMFDSDDFDQSSS

SEQ ID NO: 47 - DNA, 1032 bp, Bacteriophage P1
  CDS (1)..(1032)
atgtctaatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaa

SEQ ID NO: 48 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 49 - DNA, 20 bp, Artificial Sequence
  PCR primer
caacggcaacagtttacagg

SEQ ID NO: 50 - DNA, 20 bp, Artificial Sequence
  PCR primer
tctctggtcttcaaacatgg

SEQ ID NO: 51 - DNA, 20 bp, Artificial Sequence
  PCR primer
ttggcgcttcaccatctaac

SEQ ID NO: 52 - DNA, 20 bp, Artificial Sequence
  PCR primer
actcttctgctcgttgtgtc

SEQ ID NO: 53 - DNA, 20 bp, Artificial Sequence
  PCR primer
gtttgaccagacctttagcg

SEQ ID NO: 54 - DNA, 24 bp, Artificial Sequence
  PCR primer
cttcaaagtggtgcatgcggtgag

SEQ ID NO: 55 - DNA, 34 bp, Artificial Sequence
  PCR primer
gaaaatgcatgcaacggcaacatcaatgtccacg

SEQ ID NO: 56 - DNA, 35 bp, Artificial Sequence
  PCR primer
gaaagtcgacggtaaggcccgggaattcagcttgc

SEQ ID NO: 57 - DNA, 32 bp, Artificial Sequence
  PCR primer
ataattcccgggaacctcagggagaactttgg

SEQ ID NO: 58 - DNA, 35 bp, Artificial Sequence
  PCR primer
tataatgaattcggctactctatataatatgcttg

SEQ ID NO: 59 - DNA, 30 bp, Artificial Sequence
  PCR primer
aaaaaagagctcacattcgccacattcgac

SEQ ID NO: 60 - DNA, 31 bp, Artificial Sequence
  PCR primer
aaaaaacctgcaggtggaaggtgactcaatc

SEQ ID NO: 61 - DNA, 32 bp, Artificial Sequence
  PCR primer
aaaaaagctagccctctttgaacgagctagac

SEQ ID NO: 62 - DNA, 30 bp, Artificial Sequence
  PCR primer
aaaaaagggcccgtcgtgaacagcttgaac

SEQ ID NO: 63 - DNA, 20 bp, Artificial Sequence
  PCR primer
ggtgcttcaaaggcactatc

SEQ ID NO: 64 - DNA, 34 bp, Artificial Sequence
  PCR primer
gaaaggatccatgtctaatttacttactgttcac

SEQ ID NO: 65 - DNA, 41 bp, Artificial Sequence
  PCR primer
gaaattaattaacttaatcaccgtcttcaagtaatctaacc

SEQ ID NO: 66 - DNA, 48 bp, Artificial Sequence
  PCR primer
cacgcgtggcgcgccgcggccgcatgtccaatgttaaagtagctctac

SEQ ID NO: 67 - DNA, 45 bp, Artificial Sequence
  PCR primer
ctggcaaacctgcagggcggccgcttaacgtaccataaaattcag

SEQ ID NO: 68 - DNA, 46 bp, Artificial Sequence
  PCR primer
cacgcgtggcgcgccgcggccgcatggtcaaggtgactattttagg

SEQ ID NO: 69 - DNA, 45 bp, Artificial Sequence
  PCR primer
ctggcaaacctgcagggcggccgcttacagttttgggccatggac

SEQ ID NO: 70 - DNA, 46 bp, Artificial Sequence
  PCR primer
ctggcaaacctgcagggcggccgcttatgggttagccttgacaaag

SEQ ID NO: 71 - DNA, 41 bp, Artificial Sequence
  PCR primer
cacgcgtggcgcgccgcggccgcatgtcgcaaagaaaattc

SEQ ID NO: 72 - DNA, 48 bp, Artificial Sequence
  PCR primer
ctggcaaacctgcagggcggccgcttatgccttagtttcaacaggaac

SEQ ID NO: 73 - DNA, 36 bp, Artificial Sequence
  PCR primer
gtttaaacctactatgtacactgtataagtaaaaag

SEQ ID NO: 74 - DNA, 47 bp, Artificial Sequence
  PCR primer
gagctcagcggccgctcatatgggcgtgttctttttaattattgctc

SEQ ID NO: 75 - DNA, 46 bp, Artificial Sequence
  PCR primer
catatgagcggccgctgagctctggacacatacacattatcaaatg

SEQ ID NO: 76 - DNA, 36 bp, Artificial Sequence
  PCR primer
gtttaaacctttcaaaaccaaagttggtaaacatac

SEQ ID NO: 77 - DNA, 33 bp, Artificial Sequence
  PCR primer
gtttaaacgtacgactcatggggctttacaaac

SEQ ID NO: 78 - DNA, 53 bp, Artificial Sequence
  PCR primer
gagctcagcggccgctcatatgaagtgttgcagttgttttcttcttaataaag

SEQ ID NO: 79 - DNA, 50 bp, Artificial Sequence
  PCR primer
catatgagcggccgctgagctctctagagtccctctaattcttcctgttg

SEQ ID NO: 80 - DNA, 38 bp, Artificial Sequence
  PCR primer
gtttaaaccagtgatcgagcactcatttatacgagatg

SEQ ID NO: 81 - DNA, 35 bp, Artificial Sequence
  PCR primer
gtttaaaccattttaatttctattgctataatgtc

SEQ ID NO: 82 - DNA, 46 bp, Artificial Sequence
  PCR primer
gagctcagcggccgctcatatgatttttgtgttttgctgtgttttg

SEQ ID NO: 83 - DNA, 45 bp, Artificial Sequence
  PCR primer
catatgagcggccgctgagctctgacatctgaatgtaaaatgaac

SEQ ID NO: 84 - DNA, 36 bp, Artificial Sequence
  PCR primer
gtttaaacgtcaaggttttggcaagctgcctgtttg

SEQ ID NO: 85 - DNA, 40 bp, Artificial Sequence
  PCR primer
gtttaaacgaaactaagacataacaaaaaagcatgggatg

SEQ ID NO: 86 - DNA, 54 bp, Artificial Sequence
  PCR primer
gagctcagcggccgctcatatgttttttaaagcaatttaaatgaatattatttg

SEQ ID NO: 87 - DNA, 56 bp, Artificial Sequence
  PCR primer
catatgagcggccgctgagctcctttataaatggtttcatatcaatattatgactc

SEQ ID NO: 88 - DNA, 40 bp, Artificial Sequence
  PCR primer
gtttaaaccaaatggaacaatgagagatggtacgcggaac

SEQ ID NO: 89 - DNA, 35 bp, Artificial Sequence
  PCR primer
ttaatcggccaaccatacggtgggttcccagaacc

SEQ ID NO: 90 - DNA, 35 bp, Artificial Sequence
  PCR primer
ggttctgggaacccaccgtatggttggccgattaa

SEQ ID NO: 91 - DNA, 32 bp, Artificial Sequence
  PCR primer
cttctcaaaacagagcacaaaaactattgcac

SEQ ID NO: 92 - DNA, 32 bp, Artificial Sequence
  PCR primer
gtgcaatagtttttgtgctctgttttgagaag

SEQ ID NO: 93 - DNA, 42 bp, Artificial Sequence
  PCR primer
cacgcgtggcgcgccgcggccatgtctacccaaaacgatctg

SEQ ID NO: 94 - DNA, 46 bp, Artificial Sequence
  PCR primer
ctggcaaacctgcagggcggccttattctttagctggagcttcttc

SEQ ID NO: 95 - DNA, 23 bp, Artificial Sequence
  PCR primer
cagagagaggaagaagttggaac

SEQ ID NO: 96 - DNA, 24 bp, Artificial Sequence
  PCR primer
gtacagagaacttgtaaacaattc

SEQ ID NO: 97 - DNA, 32 bp, Artificial Sequence
  PCR primer
taatgggtacaccatacggtggattcccagag

SEQ ID NO: 98 - DNA, 32 bp, Artificial Sequence
  PCR primer
ctctgggaatccaccgtatggtgtacccatta

SEQ ID NO: 99 - DNA, 40 bp, Artificial Sequence
  PCR primer
cacgcgtggcgcgccgcggccatgcttagagccctaactc

SEQ ID NO: 100 - DNA, 41 bp, Artificial Sequence
  PCR primer
ctggcaaacctgcagggcggccttagttttgcttgacaaag

SEQ ID NO: 101 - DNA, 37 bp, Artificial Sequence
  PCR primer
cacgcgtggcgcgccgcggccatggttagcgttgcag

SEQ ID NO: 102 - DNA, 40 bp, Artificial Sequence
  PCR primer
ctggcaaacctgcagggcggccttacaactttgtctgctg

SEQ ID NO: 103 - DNA, 43 bp, Artificial Sequence
  PCR primer
cacgcgtggcgcgccgcggccatgccagcagtatcatatgatg

SEQ ID NO: 104 - DNA, 44 bp, Artificial Sequence
  PCR primer
ctggcaaacctgcagggcggccttataccaagcttagacccttg

SEQ ID NO: 105 - DNA, 46 bp, Artificial Sequence
  PCR primer
cacgcgtggcgcgccgcggccgcatgttctccagaatctctgctag

SEQ ID NO: 106 - DNA, 33 bp, Artificial Sequence
  PCR primer
gtttaaacctcggtagtgcctaccaatttaatg

SEQ ID NO: 107 - DNA, 29 bp, Artificial Sequence
  PCR primer
gtttaaacctccgttatgtatgtttgtac

SEQ ID NO: 108 - DNA, 23 bp, Artificial Sequence
  PCR primer
cacttgacttcgtcaatgaatac

SEQ ID NO: 109 - DNA, 20 bp, Artificial Sequence
  PCR primer
gtctgatctaaatggttctg

SEQ ID NO: 110 - DNA, 33 bp, Artificial Sequence
  PCR primer
gtctcagagacagagaactctatgacgcgtacg

SEQ ID NO: 111 - DNA, 33 bp, Artificial Sequence
  PCR primer
cgtacgcgtcatagagttctctgtctctgagac

SEQ ID NO: 112 - DNA, 33 bp, Artificial Sequence
  PCR primer
taccgggcgggaaggaactccagcagaagttgg

SEQ ID NO: 113 - DNA, 33 bp, Artificial Sequence
  PCR primer
ccaacttctgctggagttccttcccgcccggta

SEQ ID NO: 114 - DNA, 20 bp, Artificial Sequence
  PCR primer
ggagtgacacaacctgaaag

SEQ ID NO: 115 - DNA, 94 bp, Artificial Sequence
  PCR primer
aatattctattattatatattttcttcccaataaaacaaaataaaacaaaacacagcaaaacacaaaaatcctggaattcgcccttacatatgg

SEQ ID NO: 116 - DNA, 90 bp, Artificial Sequence
  PCR primer
tatagattgtaaagtagacgtaaagtttagtaattcattttaatgttcattttacattcagatgtcattacggctcgtgctatattcttg

SEQ ID NO: 117 - DNA, 33 bp, Artificial Sequence
  PCR primer
aaaaaaacgcgtatgtcaactgtggaagatcac

SEQ ID NO: 118 - DNA, 40 bp, Artificial Sequence
  PCR primer
aaaaaacctgcaggttaagctgctggcgcttcatctttgg

SEQ ID NO: 119 - DNA, 20 bp, Artificial Sequence
  PCR primer
aaaggctgacggacacaatc

SEQ ID NO: 120 - DNA, 90 bp, Artificial Sequence
  PCR primer
attctattattatatattttcttcccaataaaacaaaataaaacaaaacacagcaaaacacaaaaatcctggaattcgcccttacatatg

SEQ ID NO: 121 - DNA, 20 bp, Artificial Sequence
  PCR primer
gcgcatccatattttggcgg

SEQ ID NO: 122 - DNA, 32 bp, Artificial Sequence
  PCR primer
atattagtcgacccttctatcagggaagggag

SEQ ID NO: 123 - DNA, 23 bp, Artificial Sequence
  PCR primer
gtgggctacaaatgatacgatgg

SEQ ID NO: 124 - DNA, 20 bp, Artificial Sequence
  PCR primer
tcggccacttgtttattggg

SEQ ID NO: 125 - DNA, 20 bp, Artificial Sequence
  PCR primer
ctgcaggtttgccagcttac

SEQ ID NO: 126 - DNA, 22 bp, Artificial Sequence
  PCR primer
gattgtgtattagtgtatttcg

SEQ ID NO: 127 - DNA, 39 bp, Artificial Sequence
  PCR primer
catgcatgcatgtctagataaaatgttagctgctagatc

SEQ ID NO: 128 - DNA, 43 bp, Artificial Sequence
  PCR primer
catgcatgcatgttaattaacttaatcctttggaccaatcatg

SEQ ID NO: 129 - DNA, 27 bp, Artificial Sequence
  PCR primer
catcactgttaaaggaatgggtaaatc

SEQ ID NO: 130 - DNA, 23 bp, Artificial Sequence
  PCR primer
cgaaaccgttaaattcaacaatg

SEQ ID NO: 131 - DNA, 21 bp, Artificial Sequence
  PCR primer
gcatctaatgtacgttccaac

SEQ ID NO: 132 - DNA, 19 bp, Artificial Sequence
  PCR primer
cttctataggttgagaccc

SEQ ID NO: 133 - DNA, 19 bp, Artificial Sequence
  PCR primer
gagtgacacaacctgaaag

SEQ ID NO: 134 - DNA, 16 bp, Artificial Sequence
  PCR primer
gtaaaacgacggccag

SEQ ID NO: 135 - DNA, 17 bp, Artificial Sequence
  PCR primer
caggaaacagctatgac

SEQ ID NO: 136 - DNA, 31 bp, Artificial Sequence
  PCR primer
caagagtatcccatctgacaggaaccgatgg

SEQ ID NO: 137 - DNA, 1071 bp, Zygosaccharomyces rouxii
  CDS (1)..(1071)
atgcctcattctatcaacggtgatgttaaaatcgcagtattgggagctgcaggtggtattggacaatcactttcgctacttttgaagacccagttaactagagaattgccaaatcatcgtcatgctcagttagccctatacgacgtcaatgctgacgcagttcggggtgtcgcagccgacttatctcatattgatacaggtgttactgtaacaggatatgaaggtgataggatcggcgaagcgttagaaggtacggatatcgtcctgatccctgcaggtgttcctagaaaacctggtatgacaagagaagatctattggttgttaatgcaaagattgtcaagagtatagggtcatcgattgcgcagcattgcgatttaaacaaagtgttcattctactaatctcaaacccaataaattcccttgttccagtactcgttaaggaactggaatctaaatctcaaggcactcaagttgagagacgtgtgcttggtctcactaagttggattccgttagagcaagtgcatttttgcacgaggttacgattaaacatggtctaaaacctaaatctaatactcttgatgatgttccagtagttggtggtcattctggtgaaactattgtacctttattctcccaagcccctaatggtaaccgtttatcacaggacgccttggaagctcttgttcagcgtgtacaattcggaggcgatgaagtcgttagagctaaaaatggtgctggtagtgccactctgtgtatggcccatgccgcttatactgttgctgcatcttttattccacttatcactggtcaaaagcgttccatctctggtacattctatgttgccttaaaggatgctcaaggtcagcctatcaacagtagcgctaagcgtcttttgggctcaatcaacgatttaccatattttgcagtgccattggagattacttctcagggtgtggatgaattagataccagcgttttggaaagaatgaccaagtatgagagagaaagactcttagctccttgtctgggtaaattggaaggtggtatcagaaacggtttgagtttgtga

SEQ ID NO: 138 - protein, 356 aa, Zygosaccharomyces rouxii
MPHSINGDVKIAVLGAAGGIGQSLSLLLKTQLTRELPNHRHAQLALYDVNADAVRGVAADLSHIDTGVTVTGYEGDRIGEALEGTDIVLIPAGVPRKPGMTREDLLVVNAKIVKSIGSSIAQHCDLNKVFILLISNPINSLVPVLVKELESKSQGTQVERRVLGLTKLDSVRASAFLHEVTIKHGLKPKSNTLDDVPVVGGHSGETIVPLFSQAPNGNRLSQDALEALVQRVQFGGDEVVRAKNGAGSATLCMAHAAYTVAASFIPLITGQKRSISGTFYVALKDAQGQPINSSAKRLLGSINDLPYFAVPLEITSQGVDELDTSVLERMTKYERERLLAPCLGKLEGGIRNGLSL

SEQ ID NO: 139 - DNA, 939 bp, Escherichia coli
  CDS (1)..(939)
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 140 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 141 - DNA, 987 bp, Rhizopus oryzae
  CDS (1)..(987)
atggttaaagttacagtttgtggtgctgctggtggtattggtcaacccctttctttactcttgaagcaatcctctcacattactcacttatctctttatgatatcgttaatactcctggtgttgctgctgatcttagtcatatcgataccaaatccaaggtcactggtcatgtaggtgctgctcaacttgaagaagctatcaaggattctgatgttgtcgttattcccgctggtgtcccaagaaagccaggtatgacgcgtgatgatcttttcaagattaatgctggtattgtacgtgatttggctacagctgctgcaaagtacgctccaaaggccttcatgtgtatcatttctaacccagtcaactcgactgtcccaatcgttactgaagtattcaaacagcacaatgtttatgaccccaaaagaatctttggtgttacaacacttgatattgttcgtgcatccacctttgtatccgaattgattggaggtgaacctaattcacttcgtgttcccgtcattggtggtcacagcggcgtaaccatcttacctttactctcacaggtccccggcattgaaaagttaaaccaagaacaaattgagaaggtaactcatcgtattcaatttggtggcgatgaagttgtcaaggccaaggatggtgctggttctgccactctttccatggcttatgctggtgctcgttttgctacaaacatcattgaggctgcttttgctggaaagaagggcattgttgaatgtacctatgttcaattggatgctgataaatctggtgcccaatctgtcaaggatttggttggtagtgaacttgaatatttctctgttcccgttgaattgggtcctagtggtgttgaaaagattttacccattggaaacgttaatgaatatgaaaagaagttgttgaacgaggcttctcctgaattaaaaaccaacattgataaaggttgtacttttgttactgaaggctaa

SEQ ID NO: 142 - protein, 328 aa, Rhizopus oryzae
MVKVTVCGAAGGIGQPLSLLLKQSSHITHLSLYDIVNTPGVAADLSHIDTKSKVTGHVGAAQLEEAIKDSDVVVIPAGVPRKPGMTRDDLFKINAGIVRDLATAAAKYAPKAFMCIISNPVNSTVPIVTEVFKQHNVYDPKRIFGVTTLDIVRASTFVSELIGGEPNSLRVPVIGGHSGVTILPLLSQVPGIEKLNQEQIEKVTHRIQFGGDEVVKAKDGAGSATLSMAYAGARFATNIIEAAFAGKKGIVECTYVQLDADKSGAQSVKDLVGSELEYFSVPVELGPSGVEKILPIGNVNEYEKKLLNEASPELKTNIDKGCTFVTEG

(19) United States
(12) Patent Application Publication
(10) Pub. No.: US 2013/0309736 A1
(43) Pub. Date: Nov. 21, 2013

(54) COMPOSITIONS AND METHODS FOR MALATE AND FUMARATE PRODUCTION

(71) Applicants: Kenneth R. Finley, St. Bonifacius, MN (US); Jeanette M. Huryta, Excelsior, MN (US); Beth M. Mastel, Excelsior, MN (US); Thomas W. McMullin, Minnetonka, MN (US); Gregory M. Poynter, St. Paul, MN (US); Brian J. Rush, Minneapolis, MN (US); Kevin T. Watts, Minneapolis, MN (US); Arlene M. Fosmer, Eden Prairie, MN (US)

(72) Inventors: Kenneth R. Finley, St. Bonifacius, MN (US); Jeanette M. Huryta, Excelsior, MN (US); Beth M. Mastel, Excelsior, MN (US); Thomas W. McMullin, Minnetonka, MN (US); Gregory M. Poynter, St. Paul, MN (US); Brian J. Rush, Minneapolis, MN (US); Kevin T. Watts, Minneapolis, MN (US); Arlene M. Fosmer, Eden Prairie, MN (US)

(73) Assignee: CARGILL, INCORPORATED, Wayzata, MN (US)

(21) Appl. No.: 13/981,593

(22) PCT Filed: Jan. 25, 2012
(86) PCT No.: PCT/US12/22615

§ 371 Date: Jul. 24, 2013

Related U.S. Application Data
(60) Provisional application No. 61/436,138, filed on Jan. 25, 2011.

(51) Int. Cl. C12N 15/81

(52) U.S. Cl. CPC C12N 15/815
USPC 435/145; 435/254.23

(57) ABSTRACT

The present application provides genetically modified yeast cell comprising an active malate fermentation pathway and/or an active fumarate fermentation pathway, as well as methods of using these cells to produce malate and/or fumarate.

### RELATED APPLICATIONS

The present application claims priority to U.S. Provisional Patent Application No. 61/436,186, filed Jan. 25, 2011, the disclosure of which is incorporated by reference herein in its entirety.

### BACKGROUND

Malate (hydroxybutanedioc acid, hydroxyl-succinic acid) and fumarate (trans-butanedioic acid, trans-1,2,-ethylenedicarboxylic acid) are four carbon dicarboxylic acids that play key roles in the citric acid cycle. Both were recently included in a US Department of Energy list of the top 12 value added chemicals from biomass. As platform chemicals, malate and fumarate have a wide variety of potential applications including use in liquid antigels, heat transfer fluids, the solvents gamma butyrolactone (GBL) and dimethyl isosorbide, pigments, the polyesters poly-butylene succinate (PBS) and PEIT, synthesis intermediates and plasticizers. In addition, both compounds are commonly used as food acidulants. Fumarate has low water solubility (4.28 g/kg H₂O at 15.5° C.), which also makes it well suited for non-hygroscopic applications.

Malate and fumarate have traditionally been derived from maleic anhydride, which is produced by oxidation of butane. In recent years, there have been several attempts to move away from these traditional production methods to biological production methods. Biological production provides several advantages over derivation from petrochemical sources, including increased efficiency and cost effectiveness and decreased environmental impact. With regard to malate, biological production also allows for the production of pure L-enantiomer.

Previously developed biological production methods for malate and fumarate have primarily utilized bacterial or filamentous fungal fermentation hosts. Although several bacterial species have been used successfully, bacteria present certain drawbacks for large-scale organic acid production. As organic acids are produced, the fermentation medium becomes increasingly acidic. These lower pH conditions result in lower costs for organic acid production, because the resultant product is partially or wholly in the acid form. However, most bacteria do not perform well in strongly acidic environments, and therefore either die or begin producing so slowly that they become economically unviable. To prevent this, it becomes necessary to buffer the medium to maintain a higher pH. However, this makes recovery of the organic acid product more difficult and expensive. Filamentous fungi are generally more tolerant of low pH conditions than bacteria, but their high oxygen requirements often result in lower product yields and their filamentous nature can present challenges with regard to mixing and oxygen delivery. In addition, some C4-producing filamentous fungi (e.g., Aspergillus flavus) are known to produce aflotoxins, which present additional containment and purification challenges.

There has been increasing interest in recent years around the use of yeast to ferment sugars to organic acids. Yeast are used as biocatalysts in a number of industrial fermentations, and present several advantages over bacteria. While many bacteria are unable to synthesize certain amino acids or proteins that they need to grow and metabolize sugars efficiently, most yeast species can synthesize their necessary amino acids or proteins from inorganic nitrogen compounds. Yeast are also not susceptible to bacteriophage infection, which can lead to loss of productivity or of whole fermentation runs in bacteria.

Although yeast are attractive candidates for organic acid production, they present several difficulties. First, pathway engineering in yeast is typically more difficult than in bacteria. Enzymes in yeast are compartmentalized in the cytoplasm, mitochondria, or peroxisomes, whereas in bacteria they are pooled in the cytoplasm. This means that targeting signals may need to be removed in yeast to ensure that all the enzymes of the biosynthetic pathway co-exist in the same compartment within a single cell. Control of transport of pathway intermediates between the compartments may also be necessary to maximize carbon flow to the desired product. Second, not all yeast species meet the necessary criteria for economic fermentation on a large scale. In fact, only a small percentage of yeast possess the combination of sufficiently high volumetric and specific sugar utilization rates with the ability to grow robustly under low pH conditions. The Department of Energy has estimated that production rates of approximately 2.5 g/L/hour are necessary, using a minimal media, for economic fermentations of organic acid (http://www1.eere.energy.gov/biomass/pdfs/35523.pdf).

The yeast strains that have been developed thus far for malate and fumarate production have not exhibited high enough yields for economic production on an industrial scale. Therefore, there is a need for improved yeast strains that generate malate and/or fumarate on a largest scale in a more cost-effective manner.

### SUMMARY

Provided herein in certain embodiments are genetically modified yeast cells comprising an active malate fermentation pathway from phosphoenolpyruvate or pyruvate to malate. In certain embodiments, this pathway includes at least the following reactions 1) conversion of pyruvate and/or phosphoenolpyruvate to oxaloacetate and 2) conversion of oxaloacetate to malate. In certain embodiments, the pathway also includes export of malate from inside the cell to the extracellular environment. Each of the reactions in the active malate fermentation pathway is catalyzed by one or more enzymes, which in turn are encoded by one or more exogenous or endogenous malate fermentation pathway genes. In certain embodiments, all of the enzymes catalyzing reactions in the active malate fermentation pathway are encoded by endogenous genes. In other embodiments, all of the enzymes catalyzing reactions in the active malate fermentation pathway are encoded by exogenous genes. In still other embodiments, the enzymes catalyzing reactions in the active malate fermentation pathway are encoded by a mix of endogenous and exogenous genes.

In certain embodiments, the genetically modified yeast cells provided herein comprise one or more endogenous genes encoding enzymes that catalyze various reactions in the active malate fermentation pathway, and in certain of these embodiments the cells comprise one or more copies of endogenous pyruvate carboxylase, phosphoenolpyruvate carboxylase, and/or malate dehydrogenase genes. In certain of these embodiments, the endogenous genes are operatively linked to endogenous regulatory elements only. In other embodiments, one or more of the endogenous genes are operatively linked to one or more exogenous regulatory elements.

In certain embodiments, the genetically modified yeast cells provided herein comprise one or more exogenous genes encoding enzymes that catalyze various reactions in the active malate fermentation pathway, and in certain of these embodiments the cells comprise one or more copies of exogenous pyruvate carboxylase, phosphoenolpyruvate carboxylase, and/or malate dehydrogenase genes. In certain of these embodiments, the exogenous genes are operatively linked to exogenous regulatory elements only. In other embodiments, one or more of the exogenous genes are operatively linked to one or more endogenous regulatory elements.

Provided herein in certain embodiments are genetically modified yeast cells comprising an active fumarate fermentation pathway from phosphoenolpyruvate or pyruvate to fumarate. In certain embodiments, this pathway includes at least the following reactions 1) conversion of pyruvate and/or phosphoenolpyruvate to oxaloacetate, 2) conversion of oxaloacetate to malate, and 3) conversion of malate to fumarate. In certain embodiments, the pathway also includes export of fumarate from inside the cell to the extracellular environment. Each of the reactions in the active fumarate fermentation pathway is catalyzed by one or more enzymes, which in turn are encoded by one or more exogenous or endogenous fumarate fermentation pathway genes. In certain embodiments, all of the enzymes catalyzing reactions in the active fumarate fermentation pathway are encoded by endogenous genes. In other embodiments, all of the enzymes catalyzing reactions in the active fumarate fermentation pathway are encoded by exogenous genes. In still other embodiments, the enzymes catalyzing reactions in the active fumarate fermentation pathway are encoded by a mix of endogenous and exogenous genes.

In certain embodiments, the genetically modified yeast cells provided herein comprise one or more endogenous genes encoding enzymes that catalyze various reactions in the active fumarate fermentation pathway, and in certain of these embodiments the cells comprise one or more copies of endogenous pyruvate carboxylase, phosphoenolpyruvate carboxylase, malate dehydrogenase, and/or fumarase genes. In certain of these embodiments, the endogenous genes are operatively linked to endogenous regulatory elements only. In other embodiments, one or more of the endogenous genes are operatively linked to one or more exogenous regulatory elements.

In certain embodiments, the genetically modified yeast cells provided herein comprise one or more exogenous genes encoding enzymes that catalyze various reactions in the active fumarate fermentation pathway, and in certain of these embodiments the cells comprise one or more copies of exogenous pyruvate carboxylase, phosphoenolpyruvate carboxylase, malate dehydrogenase, and/or fumarase genes. In certain of these embodiments, the exogenous genes are operatively linked to exogenous regulatory elements only. In other embodiments, one or more of the exogenous genes are operatively linked to one or more endogenous regulatory elements.

In certain embodiments, the genetically modified yeast cells provided herein comprise an endogenous and/or exogenous pyruvate carboxylase gene. In certain of those embodiments where the cells comprise an exogenous pyruvate carboxylase gene, the exogenous pyruvate carboxylase gene is derived from a yeast source gene such as an I. orientalis, S. cerevisiae, or K. marxianus source gene, and in certain of these embodiments the exogenous pyruvate carboxylase gene encodes a polypeptide with at least 50%, at least 60%, at least 70%, at least 80%, at least 85%, at least 90%, at least 95%, at least 97%, or at least 99% sequence identity to a polypeptide encoded by the yeast source gene. In certain embodiments, the exogenous pyruvate carboxylase gene encodes a polypeptide that comprises the amino acid sequence of SEQ ID NOs:8, 10, or 12, or an amino acid sequence with at least 50%, at least 60%, at least 70%, at least 80%, at least 85%, at least 90%, at least 95%, at least 97%, or at least 99% sequence identity to the amino acid sequence of SEQ ID NOs:8, 10, or 12. In certain embodiments, the exogenous pyruvate carboxylase gene comprises the nucleotide sequence of SEQ ID NOs:7, 9, or 11, or a nucleotide sequence with at least 50%, at least 60%, at least 70%, at least 80%, at least 85%, at least 90%, at least 95%, at least 97%, or at least 99% sequence identity to the nucleotide sequence of SEQ ID NOs:7, 9, or 11. In certain other embodiments where the cells comprise an exogenous pyruvate carboxylase gene, the exogenous pyruvate carboxylase gene is derived from a fungal source gene other than a Rhizopus oryzae source gene.

In certain embodiments, the genetically modified yeast cells provided herein comprise an endogenous and/or exogenous phosphoenolpyruvate carboxylase gene. In certain of these embodiments where the cells comprise an exogenous phosphoenolpyruvate carboxylase gene, the exogenous phosphoenolpyruvate carboxylase gene is derived from a bacterial source gene such as an E. coli or M. succiniciproducens source gene, and in certain of these embodiments the exogenous phosphoenolpyruvate carboxylase gene encodes a polypeptide with at least 50%, at least 60%, at least 70%, at least 80%, at least 85%, at least 90%, at least 95%, at least 97%, or at least 99% sequence identity to a polypeptide encoded by the bacterial source gene. In certain embodiments, the exogenous phosphoenolpyruvate carboxylase gene encodes a polypeptide that comprises the amino acid sequence of SEQ ID NOs:4 or 6, or an amino acid sequence with at least 50%, at least 60%, at least 70%, at least 80%, at least 85%, at least 90%, at least 95%, at least 97%, or at least 99% sequence identity to the amino acid sequence of SEQ ID NOs:4 or 6. In certain embodiments, the exogenous phosphoenolpyruvate carboxylase gene comprises the nucleotide sequence of SEQ ID NOs:3 or 5, or a nucleotide sequence with at least 50%, at least 60%, at least 70%, at least 80%, at least 85%, at least 90%, at least 95%, at least 97%, or at least 99% sequence identity to the nucleotide sequence of SEQ ID NOs:3 or 5.

In certain embodiments, the genetically modified yeast cells provided herein comprise an endogenous and/or exogenous malate dehydrogenase gene. In certain of these embodiments where the cells comprise an exogenous malate dehydrogenase gene, the exogenous malate dehydrogenase gene is derived from a yeast source gene such as an I. orientalis, Zygosaccharomyces rouxii, or K. marxianus source gene, and in certain of these embodiments the exogenous malate dehydrogenase gene encodes a polypeptide with at least 50%, at least 60%, at least 70%, at least 80%, at least 85%, at least 90%, at least 95%, at least 97%, or at least 99% sequence identity to a polypeptide encoded by the yeast source gene. In certain embodiments, the exogenous malate dehydrogenase gene encodes a polypeptide that comprises the amino acid sequence of SEQ ID NOs:14, 16, 18, 138, 20, 22, or 24, or an amino acid sequence with at least 50%, at least 60%, at least 70%, at least 80%, at least 85%, at least 90%, at least 95%, at least 97%, or at least 99% sequence identity to the amino acid sequence of SEQ ID NOs:14, 16, 18, 138, 20, 22, or 24. In certain embodiments, the exogenous malate dehydrogenase gene comprises the nucleotide sequence of SEQ ID NOs:13, 15, 17, 137, 19, 21, or 23, or a nucleotide sequence with at least 50%, at least 60%, at least 70%, at least 80%, at least 85%, at least 90%, at least 95%, at least 97%, or at least 99% sequence identity to the nucleotide sequence of SEQ ID NOs:13, 15, 17, 137, 19, 21, or 23. In certain other embodiments where the cells comprise an exogenous malate dehydrogenase gene, the exogenous malate dehydrogenase gene is derived from a bacterial source gene such as an E. coli source gene, and in certain of these embodiments the exogenous malate dehydrogenase gene encodes a polypeptide with at least 50%, at least 60%, at least 70%, at least 80%, at least 85%, at least 90%, at least 95%, at least 97%, or at least 99% sequence identity to a polypeptide encoded by the bacterial source gene. In certain embodiments, the exogenous malate dehydrogenase gene encodes a polypeptide that comprises the amino acid sequence of SEQ ID NO:140, or an amino acid sequence with at least 50%, at least 60%, at least 70%, at least 80%, at least 85%, at least 90%, at least 95%, at least 97%, or at least 99% sequence identity to the amino acid sequence of SEQ ID NO:140. In certain embodiments, the exogenous malate dehydrogenase gene comprises the nucleotide sequence of SEQ ID NO:139, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:139. In certain other embodiments where the cells comprise an exogenous malate dehydrogenase gene, the exogenous malate dehydrogenase gene is derived from a fungal source gene such as a R. oryzae source gene, and in certain of these embodiments the exogenous malate dehydrogenase gene encodes a polypeptide with at least 50%, at least 60%, at least 70%, at least 80%, at least 85%, at least 90%, at least 95%, at least 97%, or at least 99% sequence identity to a polypeptide encoded by the fungal source gene. In certain embodiments, the exogenous malate dehydrogenase gene encodes a polypeptide that comprises the amino acid sequence of SEQ ID NO:142, or an amino acid sequence with at least 50%, at least 60%, at least 70%, at least 80%, at least 85%, at least 90%, at least 95%, at least 97%, or at least 99% sequence identity to the amino acid sequence of SEQ ID NO:142. In certain embodiments, the exogenous malate dehydrogenase gene comprises the nucleotide sequence of SEQ ID NO:141, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:141.

In certain embodiments, the genetically modified yeast cells provided herein comprise an endogenous and/or exogenous fumarase gene. In certain of these embodiments where the cells comprise an exogenous fumarase gene, the exogenous fumarase gene is derived from a yeast source gene such as an I. orientalis source gene, and in certain of these embodiments the exogenous fumarase gene encodes a polypeptide with at least 50%, at least 60%, at least 70%, at least 80%, at least 85%, at least 90%, at least 95%, at least 97%, or at least 99% sequence identity to a polypeptide encoded by the yeast source gene. In certain embodiments, the exogenous fumarase gene encodes a polypeptide that comprises the amino acid sequence of SEQ ID NO:2, or an amino acid sequence with at least 50%, at least 60%, at least 70%, at least 80%, at least 85%, at least 90%, at least 95%, at least 97%, or at least 99% sequence identity to the amino acid sequence of SEQ ID NO:2. In certain embodiments, the exogenous fumarase gene comprises the nucleotide sequence of SEQ ID NO:1, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:1.

In certain embodiments, the genetically modified yeast cells provided herein comprise an endogenous and/or exogenous C4 dicarboxylate exporter gene. In certain of these embodiments, the C4 dicarboxylate exporter encoded by the gene is capable of exporting malate and/or fumarate from inside the cell to the extracellular environment In certain embodiments, the genetically modified yeast cells provided herein comprise, in addition to an active malate or fumarate fermentation pathway, an active reduction pathway from glucose-6-phosphate to ribulose-5-phosphate. In certain embodiments, this pathway includes at least the following reactions 1) conversion of glucose 6-phosphate to 6-phosphogluconaolactone, 2) conversion of 6-phosphogluconaolactone to 6-phosphogluconate, and 3) conversion of 6-phosphogluconate to ribulose 5-phosphate. Each of the reactions in the active reduction pathway is catalyzed by one or more enzymes, which in turn are encoded by one or more exogenous or endogenous reduction pathway genes. In certain embodiments, all of the enzymes catalyzing reactions in the active reduction pathway are encoded by endogenous genes. In other embodiments, all of the enzymes catalyzing reactions in the active reduction pathway are encoded by exogenous genes. In still other embodiments, the enzymes catalyzing reactions in the active reduction pathway are encoded by a mix of endogenous and exogenous genes.

In certain embodiments, the genetically modified yeast cells provided herein comprise one or more endogenous genes encoding enzymes that catalyze various reactions in the active reduction pathway, and in certain of these embodiments the cells comprise one or more copies of endogenous glucose 6-phosphate dehydrogenase, gluconolactonase, and/or 6-phosphogluconate dehydrogenase genes. In certain of these embodiments, the endogenous genes are operatively linked to endogenous regulatory elements only. In other embodiments, one or more of the endogenous genes are operatively linked to one or more exogenous regulatory elements.

In certain embodiments, the genetically modified yeast cells provided herein comprise one or more exogenous genes encoding enzymes that catalyze various reactions in the active reduction pathway, and in certain of these embodiments the cells comprise one or more copies of exogenous glucose 6-phosphate dehydrogenase, gluconolactonase, and/or 6-phosphogluconate dehydrogenase genes. In certain of these embodiments, the exogenous genes are operatively linked to exogenous regulatory elements only. In other embodiments, one or more of the exogenous genes are operatively linked to one or more endogenous regulatory elements.

In certain embodiments, the genetically modified yeast cells provided herein comprise an endogenous and/or exogenous glucose 6-phosphate dehydrogenase gene. In certain of these embodiments where the cells comprise an exogenous glucose 6-phosphate dehydrogenase gene, the exogenous glucose 6-phosphate dehydrogenase gene is derived from a yeast source gene such as an I. orientalis source gene, and in certain of these embodiments the exogenous glucose 6-phosphate dehydrogenase gene encodes a polypeptide with at least 50%, at least 60%, at least 70%, at least 80%, at least 85%, at least 90%, at least 95%, at least 97%, or at least 99% sequence identity to a polypeptide encoded by the yeast source gene. In certain embodiments, the exogenous glucose 6-phosphate dehydrogenase gene encodes a polypeptide that comprises the amino acid sequence of SEQ ID NO:34, or an amino acid sequence with at least 50%, at least 60%, at least 70%, at least 80%, at least 85%, at least 90%, at least 95%, at least 97%, or at least 99% sequence identity to the amino acid sequence of SEQ ID NO:34. In certain embodiments, the exogenous glucose 6-phosphate dehydrogenase gene comprises the nucleotide sequence of SEQ ID NO:33, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:33.

In certain embodiments, the genetically modified yeast cells provided herein comprise an endogenous and/or exogenous gluconolactonase gene. In certain of these embodiments where the cells comprise an exogenous gluconolactonase gene, the exogenous gluconolactonase gene is derived from a yeast source gene such as an I. orientalis source gene, and in certain of these embodiments the exogenous gluconolactonase gene encodes a polypeptide with at least 50%, at least 60%, at least 70%, at least 80%, at least 85%, at least 90%, at least 95%, at least 97%, or at least 99% sequence identity to a polypeptide encoded by the yeast source gene. In certain embodiments, the exogenous gluconolactonase gene encodes a polypeptide that comprises the amino acid sequence of SEQ ID NO:36, or an amino acid sequence with at least 50%, at least 60%, at least 70%, at least 80%, at least 85%, at least 90%, at least 95%, at least 97%, or at least 99% sequence identity to the amino acid sequence of SEQ ID NO:36. In certain embodiments, the exogenous gluconolactonase gene comprises the nucleotide sequence of SEQ ID NO:35, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:35.

In certain embodiments, the genetically modified yeast cells provided herein comprise an endogenous and/or exogenous 6-phosphogluconate dehydrogenase gene. In certain of these embodiments where the cells comprise an exogenous 6-phosphogluconate dehydrogenase gene, the exogenous 6-phosphogluconate dehydrogenase gene is derived from a yeast source gene such as an I. orientalis source gene, and in certain of these embodiments the exogenous 6-phosphogluconate dehydrogenase gene encodes a polypeptide with at least 50%, at least 60%, at least 70%, at least 80%, at least 85%, at least 90%, at least 95%, at least 97%, or at least 99% sequence identity to a polypeptide encoded by the yeast source gene. In certain embodiments, the exogenous 6-phosphogluconate dehydrogenase gene encodes a polypeptide that comprises the amino acid sequence of SEQ ID NO:38, or an amino acid sequence with at least 50%, at least 60%, at least 70%, at least 80%, at least 85%, at least 90%, at least 95%, at least 97%, or at least 99% sequence identity to the amino acid sequence of SEQ ID NO:38. In certain embodiments, the exogenous 6-phosphogluconate dehydrogenase gene comprises the nucleotide sequence of SEQ ID NO:37, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:37.

In certain embodiments, the genetically modified yeast cells provided herein comprise a deletion or disruption of one or more endogenous genes. In certain of these embodiments, the cells comprise a deletion or disruption of an endogenous pyruvate carboxykinase, malic enzyme, and/or pyruvate decarboxylase gene.

In certain embodiments, the genetically modified yeast cells are derived from host yeast cells that exhibit a relatively high degree of malate and/or fumarate resistance. In certain embodiments, the cells provided herein belong to the genus Issatchenkia, Candida, or Saccharomyces, and in certain of these embodiments the cells belong to the Pichia fermentans/Issatchenkia orientalis clade. In certain embodiments, the cells belong to the species Issatchenkia orientalis, Candida sorbosivorans, Candida vanderwaltii, Candida guilliermondii, Candida lambica, and Saccharomyces bulderi.

Provided herein in certain embodiments are methods of producing malate and/or fumarate by culturing the genetically modified yeast cells provided herein in the presence of at least one carbon source, then isolating the malate and/or fumarate from the culture. In certain embodiments, the carbon source is one or more of glucose, xylose, arabinose, sucrose, fructose, cellulose, glucose oligomers, and glycerol.

### BRIEF DESCRIPTION OF DRAWING

FIG. 1: pMI449, CYB2A deletion construct.

FIG. 2: pMI454, CYB2A deletion construct.

FIG. 3: pBH165, GPD1 deletion construct.

FIG. 4: pCM188, GPD1 deletion construct.

FIG. 5: pCA89, CYB2B deletion construct.

FIG. 6: pMI457, PGK:MEL5 construct.

FIG. 7: pCA90, CYB2B deletion construct.

FIG. 8: pHJJ17.

FIG. 9: pVB10, PDC:cre construct.

FIG. 10: pVB32.

FIG. 11: pKF031, PGK:MEL5 construct.

FIG. 12: pKF044, CYB2A construct.

FIG. 13: pKWB18, ATO2 deletion construct.

FIG. 14: pKWB21, PDC1 deletion construct.

FIG. 15: pKF043, I. orientalis PYC1 expression construct.

FIG. 16: pKF045, I. orientalis PYC1 expression construct.

FIG. 17: pKWB14, S. cerevisiae PYC1 expression construct

FIG. 18: pKWB15, S. cerevisiae PYC1 expression construct.

FIG. 19: pKWB16, K. marxianus PYC1 expression construct.

FIG. 20: pKWB17, K. marxianus PYC1 expression construct.

FIG. 21: pGPB30, FUM1 expression constructs.

FIG. 22: pGPB42, FUM1 expression constructs.

FIG. 23: pGPB44, FUM1 expression constructs.

FIG. 24: pGPB47, FUM1 expression constructs.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference in their entirety.

### ABBREVIATIONS

α-KGDH, α-ketoglutarate dehydrogenase; CYB2, L-(+)-lactate:ferricytochrome c oxidoreductase; CYC, iso-2-cytochrome c; ENO1, enolase; FUM, fumarase; GAPDH, glyceraldehyde-3-phosphate dehydrogenase; GPD, glycerol 3-phosphate dehydrogenase; G6PD, glucose 6-phosphate dehydrogenase; IDH, isocitrate dehydrogenase; MDH, malate dehydrogenase; OAA, oxaloacetate; PDC, pyruvate decarboxylase; PEP, phosphoenolpyruvate; 6PGDH, 6-phosphogluconate dehydrogenase; PGK, phosphoglycerate kinase; PFL, pyruvate formate lyase; PPC, phosphoenolpyruvate carboxylase; PYC, pyruvate carboxylase; RKI, ribose 5-phosphate ketol-isomerase; TAL, transaldolase; TCA, tricarboxylic acid; TEF, translation elongate factor; TKL, transketolase; URA3, orotidine 5′-phosphate decarboxylase; XDH, xylitol dehydrogenase; XR, xylose reductase; 6PGDH, 6-phosphogluconate dehydrogenase.

### Description

Provided herein are genetically modified yeast cells for the production of malate and fumarate, methods of making these yeast cells, and methods of using these cells to produce malate and fumarate. “Malate” and “fumarate” as used herein includes salt and acid forms of malate and fumarate, respectively.

There are three primary fermentation pathways for producing malate and fumarate from a microorganism: reductive TCA, oxidative TCA, and glyoxylate shunt.

The reductive TCA pathway begins with carboxylation of the three carbon glycolytic intermediate phosphoenolpyruvate (PEP) or pyruvate to oxaloacetate (OAA) (by PEP carboxylase (PPC) and pyruvate carboxylase (PYC), respectively). OAA is converted to malate by malate dehydrogenase (MDH), and malate is converted to fumarate by fumarase (FUM, also known as fumarate hydratase). The net stoichiometry for malate production from this pathway is: 1 glucose (C₆H₁₂O₆)+2CO₂→2 malic acid (C₄H₆O₅). The net stoichiometry for fumarate production from this pathway is: 1 glucose (C₆H₁₂O₆)+2 CO₂→2 fumaric acid (C₄H₄O₄)+2H₂O. The reductive TCA pathway provides the highest malate and fumarate yield of the three malate/fumarate fermentation pathways.

The oxidative TCA pathway begins with the conversion of OAA and acetyl-CoA to citrate by citrate synthase. OAA can be derived from carboxylation of PEP or pyruvate, while acetyl-CoA is derived from the decarboxylation of pyruvate by PDH or pyruvate formate lyase (PFL). Citrate is converted to isocitrate by aconitase, isocitrate is converted to α-ketoglutarate by isocitrate dehydrogenase (IDH), α-ketoglutarate is converted to succinyl-CoA by α-ketoglutarate dehydrogenase (α-KGDH), succinyl-CoA is converted to succinate by succinyl coenzyme A synthetase (succinate thiokinase), succinate is converted to fumarate by succinate dehydrogenase, and fumarate is converted to malate by FUM. The net stoichiometry for malate production from this pathway is: 1 glucose (C₆H₁₂O₆)+3H₂O+6 NAD⁺>1 malic acid (C₄H₆O₅)+2 CO₂+6 (NADH+H⁺). The net stoichiometry for fumarate production from this pathway is 1 glucose (C₆H₁₂O₆)+2H₂O+6 NAD⁺→1 fumaric acid (C₄H₄O₄)+2 CO₂+6 (NADH+H⁺). This pathway has a lowest carbon yield of the three malate/fumarate fermentation pathways, but the highest yield of reducing power.

Like the oxidative TCA pathway, the glyoxylate shunt pathway begins with the generation of citrate from OAA and acetyl-CoA and the conversion of citrate to isocitrate. Isocitrate is converted to glyoxylate and succinate by isocitrate lyase. Glyoxylate is condensed with acetyl-CoA to form malate by malate synthase, while succinate is converted to fumarate or malate as discussed above with regard to the oxidative TCA pathway. The net stoichiometry for malate production from this pathway is: 1 glucose (C₆H₁₂O₆)+2H₂O+4 NAD⁺→1% malic acid (C₄H₆O₅)+⅔CO₂+4 (NADH+H⁺). The net stoichiometry for fumarate production from this pathway is: 1 glucose (C₆H₁₂O₆)+⅔H₂O+4 NAD⁺→1% fumaric acid (C₄H₄O₄)+⅔CO₂+4 (NADH+H⁺).

Several previous attempts to produce malate and fumarate from biological sources have utilized filamentous fungi. For example, the filamentous fungi Aspergillus flavus has been shown to produce relatively high yields of malate, but at very low rates. In addition, this strain is associated with aflotoxin production, which presents a special concern when producing malate for use in food applications. A commercial scale fumarate fermentation was developed in the 1940's using Rhizopus oryzae (then called Rhizopus arrhizus), and similar strains are still being used in research environments (see, e.g., WO10/147,920). Other fungal genera used for malate and/or fumarate production have included Cunninghamella and Circinella.

Bacterial hosts such as E. coli have also been used to produce malate/fumarate. However, one drawback common to bacterial hosts is relatively poor performance in acidic environments. For malate/fumarate production, allowing low pH conditions to develop as organic acids are produced is preferable for commercial production.

The ideal host for commercial malate or fumarate production should produce high levels of malate or fumarate and relatively low levels of other organic acids, and should possess a high degree of pH resistance and the ability to both grow and ferment under anaerobic or substantially anaerobic conditions.

Yeast represent an attractive candidate for malate/fumarate production due to their ability to tolerate organic acids at relatively low pH. A number of wild-type yeast strains have been shown to produce malate, including yeast strains from the genera Saccharomyces, Saccharomycodes, Zygosaccharomyces, and Schizosaccharomyces, but these wild-type strains generally produce relatively low yields and/or rates. Previous attempts to develop genetically modified yeasts for malate/fumarate production have largely utilized Saccharomyces cerevisiae. This has resulted in the development of S. cerevisiae strains that produce malate (WO07/061,590) and have been suggested as hosts for fumarate production (WO09/011,974). While S. cerevisiae is relatively tolerant to organic acids at low pH, it would be useful to develop modified yeast strains that exhibit even greater tolerance and/or higher yields.

As disclosed herein, a set of yeast cells from various species were tested for resistance to malate. Cells exhibiting malate resistance were further evaluated based on their growth rates and glucose consumption rates in media containing varying concentrations of malate. Based on these experiments, a set of ideal host cells for malate and/or fumarate production were identified. These host cells were then genetically modified to contain an active malate fermentation pathway or an active fumarate fermentation pathway, resulting in a set of genetically modified yeast cells that produce high levels of malate or fumarate under low pH conditions.

Provided herein in certain embodiments are genetically modified malate-resistant yeast cells having at least one active malate fermentation pathway from PEP or pyruvate to malate. A yeast cell having an “active malate fermentation pathway” as used herein produces active enzymes necessary to catalyze each reaction in a malate fermentation pathway, and therefore is capable of producing malate in measurable yields when cultured under fermentation conditions in the presence of at least one fermentable sugar. A yeast cell having an active malate fermentation pathway comprises one or more malate fermentation pathway genes. A “malate fermentation pathway gene” as used herein refers to the coding region of a nucleotide sequence that encodes an enzyme involved in an active malate fermentation pathway.

Provided herein in certain embodiments are genetically modified fumarate-resistant yeast cells having at least one active fumarate fermentation pathway from PEP or pyruvate to fumarate. A yeast cell having an “active fumarate fermentation pathway” as used herein produces active enzymes necessary to catalyze each reaction in a fumarate fermentation pathway, and therefore is capable of producing fumarate in measurable yields when cultured under fermentation conditions in the presence of at least one fermentable sugar. A yeast cell having an active fumarate fermentation pathway comprises one or more fumarate fermentation pathway genes. A “fumarate fermentation pathway gene” as used herein refers to the coding region of a nucleotide sequence that encodes an enzyme involved in an active fumarate fermentation pathway.

In certain embodiments, the yeast cells provided herein have a reductive TCA active malate fermentation pathway that proceeds through PEP or pyruvate and OAA intermediates. In these embodiments, the yeast cells comprise a complete set of malate fermentation pathway genes comprising at least one MDH gene and at least one PPC and/or PYC gene.

In certain embodiments, the yeast cells provided herein have a reductive TCA active fumarate fermentation pathway that proceeds through PEP or pyruvate and OAA intermediates. The reductive TCA active fumarate fermentation pathway is similar to the reductive TCA active malate fermentation pathway, but includes an extra step in which malate is converted to fumarate. In these embodiments, the yeast cells comprise a complete set of fumarate fermentation pathway genes comprising at least one FUM gene, at least one MDH gene, and at least one PPC and/or PYC gene.

In those embodiments where the yeast cells provided herein have a reductive TCA active malate and/or fumarate fermentation pathway, the cells may further have an active reduction pathway. An “active reduction pathway” as used herein produces NADH or NADPH from NAD or NADP, respectively, thereby helping to balance out redox imbalances generated by a reductive TCA pathway. A yeast cell having an active reduction pathway comprises one or more reduction pathway genes. A “reduction pathway gene” as used herein refers to the coding region of a nucleotide sequence that encodes an enzyme involved in an active reduction pathway.

In certain embodiments, the yeast cells provided herein have a pentose phosphate active reduction pathway that proceeds through glucose 6-phosphate, 6-phosphogluconaolactone, 6-phosphogluconate, and ribulose 5-phosphate intermediates. In these embodiments, the yeast cells comprise a set of reduction pathway genes comprising glucose 6-phosphate dehydrogenase (G6PD), gluconolactonase, and 6-phosphogluconate dehydrogenase (6PGDH) genes.

In certain embodiments, the yeast cells provided herein may have one or more active malate and/or fumarate fermentation pathways, or portions of such pathways, that are not reductive TCA pathways. In these embodiments, the other pathways or portions thereof may be present in addition to or in lieu of the reductive TCA pathway. For example, the cells may comprise a reductive TCA active malate fermentation pathway and all or a part of an oxidative TCA or glyoxylate shunt active malate fermentation pathway.

The malate fermentation, fumarate fermentation, and reduction pathway genes in the yeast cells provided herein may be endogenous or exogenous. “Endogenous” as used herein with regard to genetic components such as genes, promoters, and terminator sequences means that the genetic component is present at a particular location in the genome of a native form of a particular yeast cell. “Exogenous” as used herein with regard to genetic components means that the genetic component is not present at a particular location in the genome of a native form of a particular yeast cell. “Native” as used herein with regard to a yeast cell refers to a wild-type yeast cell of a particular yeast species. “Native” as used herein with regard to a metabolic pathway refers to a metabolic pathway that exists and is active in a native yeast cell.

An exogenous genetic component may have either a native or non-native sequence. An exogenous genetic component with a native sequence comprises a sequence identical to (apart from individual-to-individual mutations which do not affect function) a genetic component that is present in the genome of a native cell (i.e., the exogenous genetic component is identical to an endogenous genetic component). However, the exogenous component is present at a different location in the host cell genome than the endogenous component. For example, an exogenous MDH gene that is identical to an endogenous MDH gene may be inserted into a yeast cell, resulting in a modified cell with a non-native (increased) number of MDH gene copies. Similarly, an exogenous PDC promoter that is identical to an endogenous PDC promoter can be inserted into a yeast cell such that it is operatively linked to an endogenous gene such as an MDH gene, resulting in altered expression of the endogenous gene. An exogenous genetic component with a non-native sequence comprises a sequence that is not found in the genome of a native cell. For example, an exogenous MDH gene from a particular species may be inserted into a yeast cell of another species. Similarly, an exogenous PDC promoter from a particular species may be inserted into a yeast cell of another species.

An exogenous gene is preferably integrated into the host cell genome in a functional manner, meaning that it is capable of producing an active protein in the host cell. However, in certain embodiments the exogenous gene may be introduced into the cell as part of a vector that is stably maintained in the host cytoplasm.

In certain embodiments, the genetically modified yeast cells provided herein comprise one or more exogenous malate fermentation, fumarate fermentation, and/or reduction pathway genes. In certain embodiments, the yeast cells comprise a single exogenous gene. In other embodiments, the cells comprise multiple exogenous genes. In these embodiments, the yeast cells may comprise multiple copies of a single exogenous gene and/or copies of two or more different exogenous genes. Yeast cells comprising multiple exogenous genes may comprise any number of exogenous genes. For example, these yeast cells may comprise 1 to 20 exogenous genes, and in certain embodiments they may comprise 1 to 7 exogenous genes. Multiple copies of an exogenous gene may be integrated at a single locus such that they are adjacent to one another. Alternatively, they may be integrated at several loci within the host cell's genome.

In certain embodiments, the yeast cells provided herein comprise one or more endogenous malate fermentation, fumarate fermentation, and/or reduction pathway genes. In certain of these embodiments, the cells may be engineered to overexpress one or more of these endogenous genes, meaning that the modified cells express the endogenous gene at a higher level than a native cell under at least some conditions. In certain of these embodiments, the endogenous gene being overexpressed may be operatively linked to one or more exogenous regulatory elements. For example, one or more native or non-native exogenous strong promoters may be introduced into a cell such that they are operatively linked to one or more endogenous malate fermentation pathway genes.

In certain embodiments, the yeast cells provided herein comprise one or more endogenous malate, fumarate, and/or reduction pathway genes and one or more exogenous malate, fumarate, and/or reduction pathway genes. In these embodiments, the yeast cells may have an active malate or fumarate fermentation pathway that comprises one or more endogenous malate/fumarate fermentation pathway genes and one or more exogenous malate/fumarate fermentation pathway genes. For example, a yeast cell may comprise endogenous copies of PYC, MDH, and/or FUM genes and exogenous copies of PPC genes. In certain embodiments, the yeast cells may comprise both endogenous and exogenous copies of a single malate or fumarate fermentation pathway gene. For example, a yeast cell may comprise both endogenous and exogenous copies of an MDH gene.

Malate fermentation, fumarate fermentation, and/or reduction pathway genes in the modified yeast cells provided herein may be operatively linked to one or more regulatory elements such as a promoter or terminator. As used herein, the term “promoter” refers to an untranslated sequence located upstream (i.e., 5′) to the translation start codon of a gene (generally within about 1 to 1000 base pairs (bp), preferably within about 1 to 500 bp) which controls the start of transcription of the gene. The term “terminator” as used herein refers to an untranslated sequence located downstream (i.e., 3′) to the translation finish codon of a gene (generally within about 1 to 1000 bp, preferably within about 1 to 500 bp, and especially within about 1 to 100 bp) which controls the end of transcription of the gene. A promoter or terminator is “operatively linked” to a gene if its position in the genome relative to that of the gene is such that the promoter or terminator, as the case may be, performs its transcriptional control function. Suitable promoters and terminators are described, for example, in WO99/14335, WO00/71738, WO02/42471, WO03/102201, WO03/102152 and WO03/049525 (all incorporated by reference herein in their entirety).

Regulatory elements linked to malate fermentation, fumarate fermentation, and/or reduction pathway genes in the yeast cells provided herein may be endogenous or exogenous. For example, an endogenous malate or fumarate fermentation pathway gene may be operatively linked to only endogenous regulatory elements, or it may be linked to one or more exogenous regulatory elements. Endogenous genes operatively linked to one or more exogenous regulatory elements may exhibit higher expression levels than the same genes linked to only endogenous regulatory elements. Similarly, an exogenous malate or fumarate fermentation pathway gene may be inserted into a yeast cell such that it is operatively linked to endogenous regulatory elements only, or it may be linked to one or more exogenous regulatory elements. For example, an exogenous gene may be introduced into the cell as part of an exogenous gene expression construct that comprises one or more exogenous regulatory elements. In certain embodiments, exogenous regulatory elements, or at least the functional portions of exogenous regulatory elements, may comprise native sequences. In other embodiments, exogenous regulatory elements may comprise non-native sequences. In these embodiments, the exogenous regulatory elements may comprise a sequence with a relatively high degree of sequence identity to a native regulatory element. For example, an exogenous gene may be linked to an exogenous promoter or terminator having at least 50%, at least 60%, at least 70%, at least 80%, or at least 90% sequence identity to a native promoter or terminator. Sequence identity percentages for nucleotide or amino acid sequences can be calculated by methods known in the art, such as for example using BLAST (National Center for Biological Information (NCBI) Basic Local Alignment Search Tool) version 2.2.1 software with default parameters. For example, a sequence having an identity score of at least 90% using the BLAST version 2.2.1 algorithm with default parameters is considered to have at least 90% sequence identity. The BLAST software is available from the NCBI, Bethesda, Md. In those embodiments wherein multiple exogenous genes are inserted into a host cell, each exogenous gene may be under the control of a different regulatory element, or two or more exogenous genes may be under the control of the same regulatory elements. For example, where a first exogenous gene is linked to a first regulatory element, a second exogenous gene may also be linked to the first regulatory element, or it may be linked to a second regulatory element. The first and second regulatory elements may be identical or share a high degree of sequence identity, or they be wholly unrelated.

Examples of promoters that may be linked to one or more malate fermentation, fumarate fermentation, and/or reduction pathway genes in the yeast cells provided herein include, but are not limited to, promoters for pyruvate decarboxylase (PDC1), phosphoglycerate kinase (PGK), xylose reductase (XR), xylitol dehydrogenase (XDH), L-(+)-lactate-cytochrome c oxidoreductase (CYB2), translation elongation factor-1 or -2 (TEF1, TEF2), enolase (ENO1), glyceraldehyde-3-phosphate dehydrogenase (GAPDH), and orotidine 5′-phosphate decarboxylase (URA3) genes. In these examples, the malate fermentation, fumarate fermentation, and/or reduction pathway genes may be linked to endogenous or exogenous promoters for PDC1, PGK, XR, XDH, CYB2, TEF1, TEF2, ENO1, GAPDH, or URA3 genes. Where the promoters are exogenous, they may be identical to or share a high degree of sequence identity (i.e., at least about 80%, at least about 85%, at least about 90%, at least about 95%, or at least about 99%) with native promoters for PDC1, PGK, XR, XDH, CYB2, TEF1, TEF2, ENO1, GAPDH, or URA3 genes.

Examples of terminators that may be linked to one or more malate fermentation, fumarate fermentation, and/or reduction pathway genes in the yeast cells provided herein include, but are not limited to, terminators for PDC1, XR, XDH, transaldolase (TAL), transketolase (TKL), ribose 5-phosphate ketol-isomerase (RKI), CYB2, or iso-2-cytochrome c (CYC) genes or the galactose family of genes (especially the GAL10 terminator). In these examples, the malate fermentation, fumarate fermentation, and/or reduction pathway genes may be linked to endogenous or exogenous terminators for PDC1, XR, XDH, TAL, TKL, RKI, CYB2, or CYC genes or galactose family genes. Where the terminators are exogenous, they may be identical to or share a high degree of sequence identity (i.e., at least about 80%, at least about 85%, at least about 90%, at least about 95%, or at least about 99%) with native terminators for PDC1, XR, XDH, TAL, TKL, RKI, CYB2, or CYC genes or galactose family genes. In certain embodiments, malate fermentation, fumarate fermentation, and/or reduction pathway fermentation pathway genes are linked to a terminator that comprises a functional portion of a native GAL10 gene native to the host cell or a sequence that shares at least 80%, at least 85%, at least 90%, or at least 95% sequence identity with a native GAL10 terminator.

Exogenous genes may be inserted into a yeast host cell via any method known in the art. In preferred embodiments, the genes are integrated into the host cell genome. Exogenous genes may be integrated into the genome in a targeted or a random manner. In those embodiments where the gene is integrated in a targeted manner, it may be integrated into the loci for a particular gene, such that integration of the exogenous gene is coupled to deletion or disruption of a native gene. For example, introduction of an exogenous malate fermentation pathway gene may be coupled to deletion or disruption of one or more genes encoding enzymes involved in other fermentation product pathways. Alternatively, the exogenous gene may be integrated into a portion of the genome that does not correspond to a gene.

Targeted integration and/or deletion may utilize an integration construct. The term “construct” as used herein refers to a DNA sequence that is used to transform a cell. The construct may be, for example, a circular plasmid or vector, a portion of a circular plasmid or vector (such as a restriction enzyme digestion product), a linearized plasmid or vector, or a PCR product prepared using a plasmid or genomic DNA as a template. Methods for transforming a yeast cell with an exogenous construct are described in, for example, WO99/14335, WO00/71738, WO02/42471, WO03/102201, WO03/102152, and WO03/049525. An integration construct can be assembled using two cloned target DNA sequences from an insertion site target. The two target DNA sequences may be contiguous or non-contiguous in the native host genome. In this context, “non-contiguous” means that the DNA sequences are not immediately adjacent to one another in the native genome, but are instead are separated by a region that is to be deleted. “Contiguous” sequences as used herein are directly adjacent to one another in the native genome. Where targeted integration is to be coupled to deletion or disruption of a target gene, the integration construct may also be referred to as a deletion construct. In a deletion construct, one of the target sequences may include a region 5′ to the promoter of the target gene, all or a portion of the promoter region, all or a portion of the target gene coding sequence, or some combination thereof. The other target sequence may include a region 3′ to the terminator of the target gene, all or a portion of the terminator region, and/or all or a portion of the target gene coding sequence. Where targeted integration is not to be coupled to deletion or disruption of a native gene, the target sequences are selected such that insertion of an intervening sequence will not disrupt native gene expression. An integration or deletion construct is prepared such that the two target sequences are oriented in the same direction in relation to one another as they natively appear in the genome of the host cell. Where an integration or deletion construct is used to introduce an exogenous gene into a host cell, a gene expression cassette is cloned into the construct between the two target gene sequences to allow for expression of the exogenous gene. The gene expression cassette contains the exogenous gene, and may further include one or more regulatory sequences such as promoters or terminators operatively linked to the exogenous gene. Deletion constructs can also be constructed that do not contain a gene expression cassette. Such constructs are designed to delete or disrupt a gene sequence without the insertion of an exogenous gene.

An integration or deletion construct may comprise one or more selection marker cassettes cloned into the construct between the two target DNA sequences. The selection marker cassette contains at least one selection marker gene that allows for selection of transformants. A “selection marker gene” is a gene that encodes a protein needed for the survival and/or growth of the transformed cell in a selective culture medium, and therefore can be used to apply selection pressure to the cell. Successful transformants will contain the selection marker gene, which imparts to the successfully transformed cell at least one characteristic that provides a basis for selection. Typical selection marker genes encode proteins that (a) confer resistance to antibiotics or other toxins (e.g., resistance to bleomycin or zeomycin (e.g., Streptoalloteichus hindustanus ble gene), aminoglycosides such as G418 or kanamycin (e.g., kanamycin resistance gene from transposon Tn903), or hygromycin (e.g., aminoglycoside antibiotic resistance gene from E. coli), (b) complement auxotrophic deficiencies of the cell (e.g., deficiencies in leucine (e.g., Kluyveromyces marxianus LEU2 gene), uracil (e.g., K. marxianus, Saccharomyces cerevisiae, or Issatchenkia orientalis URA3 gene), or tryptophan (e.g., K. marxianus, S. cerevisiae, or I. orientalis TRP gene)), (c) enable the cell to synthesize critical nutrients not available from simple media, or (d) confer the ability for the cell to grow on a particular carbon source (e.g., MEL5 gene from S. cerevisiae, which encodes the alpha-galactosidase (melibiose) enzyme and confers the ability to grow on melibiose as the sole carbon source). Preferred selection markers include the URA3 gene, zeocin resistance gene, G418 resistance gene, MEL5 gene, and hygromycin resistance gene. Another preferred selection marker is a CYB2 gene cassette, provided that the host cell either natively lacks such a gene or that its native CYB2 gene(s) are first deleted or disrupted. A selection marker gene is operatively linked to one or more promoter and/or terminator sequences that are operable in the host cell. In certain embodiments, these promoter and/or terminator sequences are exogenous promoter and/or terminator sequences that are included in the selection marker cassette. Suitable promoters and terminators are as described above.

An integration or deletion construct is used to transform the host cell. Transformation may be accomplished using, for example, electroporation and/or chemical transformation (e.g., calcium chloride, lithium acetate-based, etc.) methods. Selection or screening based on the presence or absence of the selection marker may be performed to identify successful transformants. In successful transformants, a homologous recombination event at the locus of the target site results in the disruption or the deletion of the target site sequence. Where the construct targets a native gene for deletion or disruption, all or a portion of the native target gene, its promoter, and/or its terminator may be deleted during this recombination event. The expression cassette, selection marker cassette, and any other genetic material between the target sequences in the integration construct is inserted into the host genome at the locus corresponding to the target sequences. Analysis by PCR or Southern analysis can be performed to confirm that the desired insertion/deletion has taken place.

In some embodiments, cell transformation may be performed using DNA from two or more constructs, PCR products, or a combination thereof, rather than a single construct or PCR product. In these embodiments, the 3′ end of one integration fragment overlaps with the 5′ end of another integration fragment. In one example, one construct will contain the first sequence from the locus of the target sequence and a non-functional part of the marker gene cassette, while the other will contain the second sequence from the locus of the target sequence and a second non-functional part of the marker gene cassette. The parts of the marker gene cassette are selected such that they can be combined to form a complete cassette. The cell is transformed with these pieces simultaneously, resulting in the formation of a complete, functional marker or structural gene cassette. Successful transformants can be selected for on the basis of the characteristic imparted by the selection marker. In another example, the selection marker resides on one fragment but the target sequences are on separate fragments, so that the integration fragments have a high probability of integrating at the site of interest. In other embodiments, transformation from three linear DNAs can be used to integrate exogenous genetic material. In these embodiments, one fragment overlaps on the 5′ end with a second fragment and on the 3′ end with a third fragment.

An integration or deletion construct may be designed such that the selection marker gene and some or all of its regulatory elements can become spontaneously deleted as a result of a subsequent homologous recombination event. A convenient way of accomplishing this is to design the construct such that the selection marker gene and/or regulatory elements are flanked by repeat sequences. Repeat sequences are identical DNA sequences, native or non-native to the host cell, and oriented on the construct in the same direction with respect to one another. The repeat sequences are advantageously about 25 to 1500 bp in length, and do not have to encode for anything. Inclusion of the repeat sequences permits a homologous recombination event to occur, which results in deletion of the selection marker gene and one of the repeat sequences. Since homologous recombination occurs with relatively low frequency, it may be necessary to grow transformants for several rounds on nonselective media to allow for the spontaneous homologous recombination to occur in some of the cells. Cells in which the selection marker gene has become spontaneously deleted can be selected or screened on the basis of their loss of the selection characteristic imparted by the selection marker gene. In certain cases, expression of a recombinase enzyme may enhance recombination between the repeated sites.

An exogenous malate fermentation, fumarate fermentation, or reduction pathway gene in the modified yeast cells provided herein may be derived from a source gene from any suitable source organism. For example, an exogenous gene may be derived from a yeast, fungal, bacterial, plant, insect, or mammalian source. As used herein, an exogenous gene that is “derived from” a source gene encodes a polypeptide that 1) is identical to a polypeptide encoded by the source gene, 2) shares at least 50%, at least 60%, at least 70%, at least 80%, at least 85%, at least 90%, at least 95%, at least 97%, or at least 99% sequence identity with a polypeptide encoded by the source gene, and/or 3) has the same function in a malate fermentation, fumarate fermentation, or reduction pathway as the polypeptide encoded by the source gene. For example, a FUM gene that is derived from an I. orientalis FUM gene may encode a polypeptide comprising the amino acid sequence of SEQ ID NO:2, a polypeptide with at least 50%, at least 60%, at least 70%, at least 80%, at least 85%, at least 90%, at least 95%, at least 97%, or at least 99% sequence identity to the amino acid sequence of SEQ ID NO:2, and/or a polypeptide that has the ability to catalyze the conversion of malate to fumarate. A gene derived from a source gene may comprise a nucleotide sequence with at least 50%, at least 60%, at least 70%, at least 80%, at least 85%, at least 90%, at least 95%, at least 97%, or at least 99% sequence identity to the coding region of the source gene. In certain embodiments, a gene derived from a source gene may comprise a nucleotide sequence that is identical to the coding region of the source gene. For example, a FUM gene that is derived from an I. orientalis FUM gene may comprise the nucleotide sequence of SEQ ID NO:1 or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:1.

In certain embodiments of the modified yeast cells provided herein, an exogenous malate fermentation, fumarate fermentation pathway, and/or reduction pathway gene may be derived from the host yeast species. For example, where the host cell is I. orientalis, an exogenous gene may be derived from a native I. orientalis gene. In these embodiments, the exogenous gene may comprise a nucleotide sequence identical to the coding region of the native gene, such that incorporation of the exogenous gene into the host cell increases the copy number of a native gene sequence and/or changes the regulation or expression level of the gene if under the control of a promoter that is different from the promoter that drives expression of the gene in a wild-type cell. In other embodiments, the exogenous gene may comprise a nucleotide sequence that differs from the coding region of a native gene, but nonetheless encodes a polypeptide that is identical to the polypeptide encoded by the native gene. In still other embodiments, the exogenous gene may comprise a nucleotide sequence that encodes a polypeptide with at least 50%, at least 60%, at least 70%, at least 80%, at least 85%, at least 90%, at least 95%, at least 97%, or at least 99% sequence identity to a polypeptide encoded by one or more native genes. In certain of these embodiments, the exogenous gene comprises a nucleotide sequence with at least 50%, at least 60%, at least 70%, at least 80%, at least 85%, at least 90%, at least 95%, at least 97%, or at least 99% sequence identity to the coding region of one or more native genes. In still other embodiments, the exogenous gene may encode a polypeptide that has less than 50% sequence identity to a polypeptide encoded by a native gene, but which nonetheless has the same function as the native polypeptide in an active malate fermentation, fumarate fermentation, or reduction pathway (i.e., the ability to catalyze the same reaction between reaction intermediates).

In other embodiments, an exogenous malate fermentation, fumarate fermentation, or reduction pathway gene may be derived from a species that is different than that of the host yeast cell. In certain of these embodiments, the exogenous gene may be derived from a different yeast species than the host cell. For example, where the host cell is I. orientalis, the exogenous gene may be derived from S. cerevisiae. In other embodiments, the exogenous gene may be derived from a fungal, bacterial, plant, insect, or mammalian source. For example, where the host cell is I. orientalis, the exogenous gene may be derived from a bacterial source such as E. coli. In those embodiments where the exogenous gene is derived from a non-yeast source, the exogenous gene sequence may be codon optimized for expression in a yeast host cell.

In those embodiments where the exogenous malate fermentation, fumarate fermentation, or reduction pathway gene is derived from a species other than the host cell species, the exogenous gene may encode a polypeptide identical to a polypeptide encoded by a native gene from the source organism. In certain of these embodiments, the exogenous gene may be identical to a native gene from the source organism. In other embodiments, the exogenous gene may share at least 50%, at least 60%, at least 70%, at least 80%, at least 85%, at least 90%, at least 95%, at least 97%, or at least 99% sequence identity to the coding region of a native gene from the source organism. In other embodiments, the exogenous gene may encode a polypeptide that shares at least 50%, at least 60%, at least 70%, at least 80%, at least 85%, at least 90%, at least 95%, at least 97%, or at least 99% sequence identity with a polypeptide encoded by a native gene from the source organism. In certain of these embodiments, the exogenous gene may comprise a nucleotide sequence with at least 50%, at least 60%, at least 70%, at least 80%, at least 85%, at least 90%, at least 95%, at least 97%, or at least 99% sequence identity to the coding region of one or more native genes from the source organism. In still other embodiments, the exogenous gene may encode a polypeptide that has less than 50% sequence identity to a polypeptide encoded by a native gene from the source organism, but which nonetheless has the same function as the native polypeptide from the source organism in an active malate fermentation, fumarate fermentation, or active reduction pathway. An exogenous source gene may be subjected to mutagenesis if necessary to provide a coding sequence starting with the usual eukaryotic starting codon (ATG), or for other purposes.

In certain embodiments, the genetically modified yeast cells provided herein have a reductive TCA active malate fermentation pathway that proceeds via PEP or pyruvate and OAA intermediates. In these embodiments, the cells comprise one or more malate fermentation pathway genes encoding enzymes selected from the group consisting PPC, PYC, and MDH genes. In certain embodiments, the cells also have one or more active reduction pathways. In these embodiments, the cells comprise one or more reduction pathway genes encoding enzymes selected from the group consisting of G6PD, gluconolactonase, and 6PGDH. In certain embodiments, the cells may comprise all or part of an active oxidative TCA or glyoxylate shunt malate or fumarate fermentation pathway. In these embodiments, the cells comprise one or more genes encoding enzymes selected from the group consisting of citrate synthase, PDH, PFL, aconitase, IDH, α-KGDH, succinate thiokinase, isocitrate lyase, and malate synthase.

In certain embodiments, the genetically modified yeast cells provided herein have a reductive TCA active fumarate fermentation pathway that proceeds via PEP or pyruvate, OAA, and malate intermediates. In these embodiments, the cells comprise one or more fumarate fermentation pathway genes encoding enzymes selected from the group consisting PPC, PYC, MDH, and FUM genes. In certain embodiments, the cells also have one or more active reduction pathways. In these embodiments, the cells comprise one or more reduction pathway genes encoding enzymes selected from the group consisting of G6PD, gluconolactonase, and 6PGDH. In certain embodiments, the cells may comprise all or part of an active oxidative TCA or glyoxylate shunt malate or fumarate fermentation pathway. In these embodiments, the cells comprise one or more genes encoding enzymes selected from the group consisting of citrate synthase, PDH, PFL, aconitase, IDH, α-KGDH, succinate thiokinase, isocitrate lyase, and malate synthase.

A “PEP carboxylase gene” or “PPC gene” as used herein refers to any gene that encodes a polypeptide with PEP carboxylase activity, meaning the ability to catalyze the conversion of PEP to OAA. In certain embodiments, a PPC gene may be derived from a bacterial source. For example, a PPC gene may be derived from an E. coli PPC gene encoding the amino acid sequence set forth in SEQ ID NO:4 or a Mannheimia succiniciproducens PPC gene encoding the amino acid sequence set forth in SEQ ID NO:6. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NOs:4 or 6. In certain embodiments, a PYC gene may comprise the nucleotide sequence set forth in SEQ ID NOs:3 or 5, or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NOs:3 or 5. In other embodiments, a PPC gene may be derived from a plant source.

A “pyruvate carboxylase gene” or “PYC gene” as used herein refers to any gene that encodes a polypeptide with pyruvate carboxylase activity, meaning the ability to catalyze the conversion of pyruvate to OAA. In certain embodiments, a PYC gene may be derived from a yeast source. For example, the PYC gene may be derived from an I. orientalis PYC gene encoding the amino acid sequence set forth in SEQ ID NO:8, an S. cerevisiae PYC1 gene encoding the amino acid sequence set forth in SEQ ID NO:10, or a K. marxianus PYC1 gene encoding the amino acid sequence set forth in SEQ ID NO:12. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NOs:8, 10, or 12. In certain embodiments, a PYC gene may comprise the nucleotide sequence set forth in SEQ ID NOs:7, 9, or 11, or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NOs:7, 9, or 11. In other embodiments, a PYC gene may be derived from a fungal source other than R. oryziae.

A “malate dehydrogenase gene” or “MDH gene” as used herein refers to any gene that encodes a polypeptide with malate dehydrogenase activity, meaning the ability to catalyze the conversion of OAA to malate. In certain embodiments, an MDH gene may be derived from a yeast source. For example, the MDH gene may be derived from an I. orientalis MDH1, MDH2, or MDH3 gene encoding the amino acid sequence set forth in SEQ ID NOs:14, 16, or 18, respectively, a Z. rouxii MDH gene encoding the amino acid sequence set forth in SEQ ID NO:138, or a K. marxianus MDH1, MDH2, or MDH3 gene encoding the amino acid sequence set forth in SEQ ID NOs:20, 22, or 24, respectively. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NOs:14, 16, 18, 138, 20, 22, or 24. In certain embodiments, a yeast-derived MDH gene may comprise the nucleotide sequence set forth in SEQ ID NOs:13, 15, 17, 137, 19, 21, or 23 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NOs:13, 15, 17, 137, 19, 21, or 23. In certain embodiments, an MDH gene may be derived from a bacterial source. For example, the MDH gene may be derived from an E. coli MDH gene encoding the amino acid sequence set forth in SEQ ID NO:140. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:140. In certain embodiments, a bacterial-derived MDH gene may comprise the nucleotide sequence set forth in SEQ ID NO:139 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO:139. In certain embodiments, an MDH gene may be derived from a fungal source. For example, the MDH gene may be derived from an R. oryzae MDH gene encoding the amino acid sequence set forth in SEQ ID NO:142. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:142. In certain embodiments, a fungal-derived MDH gene may comprise the nucleotide sequence set forth in SEQ ID NO:141 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO:141.

A “fumarase gene” or “FUM gene” as used herein refers to any gene that encodes a polypeptide with fumarase activity, meaning the ability to catalyze the conversion of malate to fumarate. In certain embodiments, a FUM gene may be derived from a yeast source. For example, the FUM gene may be derived from an I. orientalis FUM gene encoding the amino acid sequence set forth in SEQ ID NO:2. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:2. In certain embodiments, a FUM gene may comprise the nucleotide sequence set forth in SEQ ID NO:1 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO:1. In other embodiments, a FUM gene may be derived from a bacterial source outside of the Mannheimia genus.

A “glucose 6-phosphate dehydrogenase” or “G6PD gene” as used herein refers to any gene that encodes a polypeptide with glucose 6-phosphate dehydrogenase activity, meaning the ability to catalyze the conversion of glucose 6-phosphate to 6-phosphogluconolactone. In certain embodiments, a G6PD gene may be derived from a yeast source. For example, the G6PD gene may be derived from an I. orientalis G6PD gene (ZWF1) encoding the amino acid sequence set forth in SEQ ID NO:26. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:26. In certain embodiments, a G6PD gene may comprise the nucleotide sequence set forth in SEQ ID NO:25 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO:25.

A “gluconolactonase gene” as used herein refers to any gene that encodes a polypeptide with gluconolactonase activity, meaning the ability to catalyze the conversion of 6-phosphogluconolactone to 6-phosphogluconate. In certain embodiments, a gluconolactonase gene may be derived from a yeast source. For example, the gluconolactonase gene may be derived from an I. orientalis gluconolactonase gene encoding the amino acid sequence set forth in SEQ ID NO:28. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:28. In certain embodiments, a gluconolactonase gene may comprise the nucleotide sequence set forth in SEQ ID NO:27 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO:27.

A “6-phosphogluconate dehydrogenase gene” or “6PGDH gene” as used herein refers to any gene that encodes a polypeptide with 6-phosphogluconate dehydrogenase activity, meaning the ability to catalyze the conversion of 6-phosphogluconate to ribulose-5-phosphate. In certain embodiments, a 6PGDH gene may be derived from a yeast source. For example, the 6PGDH gene may be derived from an I. orientalis 6PGDH gene encoding the amino acid sequence set forth in SEQ ID NO:30. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:30. In certain embodiments, a 6PGDH gene may comprise the nucleotide sequence set forth in SEQ ID NO:29 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO:29.

In certain embodiments, the genetically modified yeast cells provided herein further comprise a deletion or disruption of one or more native genes. “Deletion or disruption” with regard to a native gene means that either the entire coding region of the gene is eliminated (deletion) or the coding region of the gene, its promoter, and/or its terminator region is modified (such as by deletion, insertion, or mutation) such that the gene no longer produces an active enzyme, produces a severely reduced quantity (at least 75% reduction, preferably at least 90% reduction) of an active enzyme, or produces an enzyme with severely reduced (at least 75% reduced, preferably at least 90% reduced) activity.

In certain embodiments, deletion or disruption of one or more native genes results in a deletion or disruption of one or more native metabolic pathways. “Deletion or disruption” with regard to a metabolic pathway means that the pathway is either inoperative or else exhibits activity that is reduced by at least 75%, at least 85%, or at least 95% relative to the native pathway. In certain embodiments, deletion or disruption of a native metabolic pathway is accomplished by incorporating one or more genetic modifications that result in decreased expression of one or more native genes that reduce malate or fumarate production.

In certain embodiments, deletion or disruption of native gene can be accomplished by forced evolution, mutagenesis, or genetic engineering methods, followed by appropriate selection or screening to identify the desired mutants. In certain embodiments, deletion or disruption of a native host cell gene may be coupled to the incorporation of one or more exogenous genes into the host cell, i.e., the exogenous genes may be incorporated using a gene expression integration construct that is also a deletion construct. In other embodiments, deletion or disruption may be accomplished using a deletion construct that does not contain an exogenous gene or by other methods known in the art.

In certain embodiments, the modified yeast cells provided herein comprise a deletion of one or more native genes encoding an enzyme involved in the conversion of the desired end product to a different product. For example, where the cells contain an active malate fermentation pathway, the cells may comprise a deletion or disruption of a FUM gene in order to decrease or prevent conversion of malate to fumarate. Similarly, cells containing an active malate fermentation pathway or an active fumarate fermentation pathway may comprise a deletion or disruption of a fumarate reductase (FRD) gene, which encodes an enzyme that converts fumarate to succinate.

In certain embodiments, the modified yeast cells provided herein comprise a deletion or disruption of one or more native genes encoding an enzyme involved in ethanol fermentation or consumption, including for example pyruvate decarboxylase (PDC, catalyzes the conversion of pyruvate to acetaldehyde) and/or alcohol dehydrogenase 1 (ADH1, catalyzes the conversion of acetaldehyde to ethanol) or 2 (ADH2, catalyzes the conversion of ethanol to acetaldehyde). Such modifications decrease the ability of the yeast cell to produce ethanol, thereby maximizing malate and/or fumarate production. In certain embodiments wherein the modified yeast cell is I. orientalis, the cells comprise a deletion of a PDC gene encoding the amino acid sequence of SEQ ID NO:32, an ADHa gene encoding the amino acid sequence of SEQ ID NO:34, and/or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NOs:32 or 34. In certain of these embodiments, the deleted gene may comprise the nucleotide sequence of SEQ ID NOs:31 or 33, or a nucleotide sequence with at least 50%, at least 60%, at least 70%, at least 80%, at least 85%, at least 90%, at least 95%, at least 97%, or at least 99% sequence identity to the nucleotide sequence of SEQ ID NOs:31 or 33.

In certain embodiments, the modified yeast cells provided herein comprise a deletion or disruption of one or more native genes encoding an enzyme involved in producing alternate fermentative products such as glycerol or other by-products such as acetate or diols, including for example glycerol 3-phosphate dehydrogenase (GPD, catalyzes the conversion of dihydroxyacetone phosphate to glycerol 3-phosphate). In certain embodiments wherein the modified yeast cell is I. orientalis, the cells comprise a deletion of a GPD gene encoding the amino acid sequence of SEQ ID NO:36 or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NO:36. In certain of these embodiments, the deleted GPD gene may comprise the nucleotide sequence of SEQ ID NO:35 or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:35.

In certain embodiments, the modified yeast cells provided herein comprise a deletion or disruption of one or more native genes encoding an enzyme that catalyzes a reverse reaction in a malate or fumarate fermentation pathway. For example, in certain embodiments the modified yeast cells provided herein comprise a deletion or disruption of a native PEP carboxykinase (PCK) gene, which encodes an enzyme that converts OAA to PEP. In certain embodiments wherein the modified yeast cell is I. orientalis, the cells comprise a deletion of a PCK gene encoding the amino acid sequence of SEQ ID NO:38 or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NO:38. In certain of these embodiments, the deleted PCK gene may comprise the nucleotide sequence of SEQ ID NO:37 or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:37. In another example, the modified yeast cells provided herein comprise a deletion or disruption of a native malic enzyme (MAE) gene, which encodes an enzyme that converts malate to pyruvate. In certain embodiments wherein the modified yeast cell is I. orientalis, the cells comprise a deletion of an MAE gene encoding the amino acid sequence of SEQ ID NO:40 or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NO:40. In certain of these embodiments, the deleted MAE gene may comprise the nucleotide sequence of SEQ ID NO:39 or a nucleotide sequence with at least 50%, at least 60%, at least 70%, at least 80%, at least 85%, at least 90%, at least 95%, at least 97%, or at least 99% sequence identity to the nucleotide sequence of SEQ ID NO:39.

In certain embodiments, the genetically modified yeast cells provided herein comprise a deletion or disruption of one or more native genes encoding an enzyme involved in an undesirable reaction with a malate or fumarate fermentation pathway product or intermediate.

In certain embodiments, the genetically modified yeast cells provided herein comprise a deletion or disruption of one or more native genes encoding an enzyme that has a neutral effect on a malate or fumarate fermentation pathway, including for example native genes encoding an enzyme selected from the group consisting of ammonia transport outward (ATO) and L-lactate cytochrome-c oxidoreductase (CYB2A or CYB2B, catalyzes the conversion of lactate to pyruvate). Deletion or disruption of neutral genes allows for insertion of one or more exogenous genes without affecting native fermentation pathways. In certain embodiments wherein the modified yeast cell is I. orientalis, the cells comprise a deletion of a CYB2A gene encoding the amino acid sequence of SEQ ID NO:42, a CYB2B gene encoding the amino acid sequence of SEQ ID NO:44, an ATO2 gene encoding the amino acid sequence of SEQ ID NO:46 and/or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NOs:42, 44, or 46. In certain of these embodiments, the deleted gene may comprise the nucleotide sequence of SEQ ID NOs:41, 43, or 45 or a nucleotide sequence with at least 50%, at least 60%, at least 70%, at least 80%, at least 85%, at least 90%, at least 95%, at least 97%, or at least 99% sequence identity to the nucleotide sequence of SEQ ID NOs:41, 43, or 45.

In certain embodiments, the genetically modified yeast cells provided herein are malate resistant yeast cells. A “malate-resistant yeast cell” as used herein refers to a yeast cell that exhibits a growth rate in media containing 200 g/L or greater malate at pH 2.85 that is at least 50% as high as its growth rate in the same media containing 0 g/L malate. In certain of these embodiments, the yeast cells may exhibit malate resistance in their native form. In other embodiments, the cells may have undergone mutation and/or selection before, during, or after introduction of genetic modifications related to an active malate fermentation pathway, such that the mutated and/or selected cells possess a higher degree of resistance to malate than wild-type cells of the same species. In certain embodiments, mutation and/or selection may be carried out on cells that exhibit malate resistance in their native form. Cells that have undergone mutation and/or selection may be tested for sugar consumption and other characteristics in the presence of varying levels of malate in order to determine their potential as industrial hosts for malate production.

In certain embodiments, the genetically modified yeast cells provided herein are fumarate resistant yeast cells. A “fumarate-resistant yeast cell” as used herein refers to a yeast cell that exhibits a growth rate in media containing fumarate that is greater than the growth rate exhibited by S. cerevisiae strains CEN.PK 111-61A or CEN.PK 182 in the same media. In certain of these embodiments, the yeast cells may exhibit fumarate resistance in their native form. In other embodiments, the cells may have undergone mutation and/or selection before, during, or after introduction of genetic modifications related to an active fumarate fermentation pathway, such that the mutated and/or selected cells possess a higher degree of resistance to fumarate than wild-type cells of the same species. In certain embodiments, mutation and/or selection may be carried out on cells that exhibit fumarate resistance in their native form. Cells that have undergone mutation and/or selection may be tested for sugar consumption and other characteristics in the presence of varying levels of fumarate in order to determine their potential as industrial hosts for fumarate production.

In certain embodiments, the genetically modified yeast cells provided herein exhibit both malate and fumarate resistance.

Selection for resistance to malate, fumarate, or other compounds may be accomplished using methods well known in the art. For example, selection may be carried out using a chemostat. A chemostat is a device that allows for a continuous culture of microorganisms (e.g., yeast) wherein the specific growth rate and cell number can be controlled independently. A continuous culture is essentially a flow system of constant volume to which medium is added continuously and from which continuous removal of any overflow can occur. Once such a system is in equilibrium, cell number and nutrient status remain constant, and the system is in a steady state. A chemostat allows control of both the population density and the specific growth rate of a culture through dilution rate and alteration of the concentration of a limiting nutrient, such as a carbon or nitrogen source. By altering the conditions as a culture is grown (e.g., decreasing the concentration of a secondary carbon source necessary to the growth of the inoculum strain, among others), microorganisms in the population that are capable of growing faster at the altered conditions will be selected and will outgrow microorganisms that do not function as well under the new conditions. Typically such selection requires the progressive increase or decrease of at least one culture component over the course of growth of the chemostat culture. The operation of chemostats and their use in the directed evolution of microorganisms is well known in the art (see, e.g., Novick Proc Natl Acad Sci USA 36:708-719 (1950), Harder J Appl Bacteriol 43:1-24 (1977).

As disclosed herein, yeast strains exhibiting malate resistance were identified based on their growth rate and glucose consumption rates in malate containing media. One such malate resistant strain was I. orientalis strain CD1822. Strain CD1822 was generated by evolving I. orientalis ATCC PTA-6658 for 91 days in a glucose limited chemostat. The system was fed with 15 g/L dextrose in a DM medium, and operated at a dilution rate of 0.06 h⁻¹ at pH=3 with added lactic acid in the feed medium. The conditions were maintained with a low oxygen transfer rate of approximately 2 mmol L⁻¹h⁻¹, and dissolved oxygen concentration remained constant at 0% of air saturation. Single colony isolates from the final time point were characterized in two shake flask assays. In the first assay, the strains were characterized for their ability to ferment glucose to ethanol in the presence of 25 g/L total lactic acid with no pH adjustment in the DM defined medium. In the second assay, the growth rate of the isolates were measured in the presence of 25, 32 and 45 g/L of total lactic, with no pH adjustment in DM defined medium. Strain CD1822 was a single isolate selected based on the measured fermentation rates and growth rates.

Yeast strains exhibiting the best combinations of growth and glucose consumption in malate media as disclosed in the examples below are preferred host cells for various genetic modifications relating to malate fermentation pathways. Yeast genera that possess the potential for a high degree of malate resistance, as indicated by growth in the presence of 200 g/L malate at a pH of 2.85, include for example Issatchenkia, Candida, and Saccharomyces. Species exhibiting a high degree of malate resistance included I. orientalis (also known as Candida krusei), Candida sorbosivorans, Candida vanderwaltii, Candida guilliermondii, Candida lambica (also known as Pichia fermentans), and Saccharomyces bulderi. I. orientalis and C. lambica belong to the I. orientalis/P. fermentans clade. Specific strains exhibiting malate resistance included I. orientalis strains PTA-6658, CD1822, 60585, and 24210, C. sorbosivorans strains CD1978 and 38619, C. vanderwaltii strain MUCL 30000, C. guilliermondii strain 20118, X. lambica strain 38617, and S. bulderi strain MYA-404.

Other wild-type yeast or fungi may be tested in a similar manner and identified to have acceptable levels of growth and glucose utilization in the presence of high levels of malate and/or fumarate as described herein. For example, Gross and Robbins (Hydrobiologia 433(103):91-109) have compiled a list of 81 fungal species identified in low pH (<4) environments that could be relevant to test as potential production hosts.

In certain embodiments, the modified yeast cells provided herein are generated by incorporating one or more genetic modifications into a Crabtree-negative host yeast cell. In certain of these embodiments the host yeast cell belongs to the genus Issatchenkia or Candida, and in certain of these embodiments the host cell belongs to the I. orientalis/P. fermentans clade. In certain of embodiments, the host cell is I. orientalis, C. sorbosivorans, C. vanderwaltii, C. guilliermondii, or C. lambica.

The I. orientalis/P. fermentans clade is the most terminal clade that contains at least the species I. orientalis, Pichia galeiformis, Pichia sp. YB-4149 (NRRL designation), Candida ethanolica, Pichia deserticola, Pichia membranifaciens, and P. fermentans. Members of the I. orientalis/P. fermentans clade are identified by analysis of the variable D1/D2 domain of the 26S ribosomal DNA of yeast species, using the method described by Kurtzman and Robnett in “Identification and Phylogeny of Ascomycetous Yeasts from Analysis of Nuclear Large Subunit (26S) Ribosomal DNA Partial Sequences,” Antonie van Leeuwenhoek 73:331-371, 1998, incorporated herein by reference (see especially p. 349). Analysis of the variable D1/D2 domain of the 26S ribosomal DNA from hundreds of ascomycetes has revealed that the I. orientalis/P. fermentans clade contains very closely related species. Members of the I. orientalis/P. fermentans clade exhibit greater similarity in the variable D1/D2 domain of the 26S ribosomal DNA to other members of the clade than to yeast species outside of the clade. Therefore, other members of the I. orientalis/P. fermentans clade can be identified by comparison of the D1/D2 domains of their respective ribosomal DNA and comparing to that of other members of the clade and closely related species outside of the clade, using Kurtzman and Robnett's methods.

In certain embodiments, the genetically modified yeast cells provided herein belong to the genus Issatchenkia, and in certain of these embodiments the yeast cells are I. orientalis. When first characterized, the species I. orientalis was assigned the name Pichia kudriavzevii. The anamorph (asexual form) of I. orientalis is known as C. krusei. Numerous additional synonyms for the species I. orientalis have been listed elsewhere (Kurtzman and Fell, The Yeasts, a Taxonomic Study. Section 35. Issatchenkia Kudryavtsev, pp 222-223 (1998)).

The ideal yeast cell for malate and/or fumarate production is ideally capable of conducting fermentation at low pH levels. The ability to conduct fermentation at a low pH decreases downstream recovery costs, resulting in more economical production. Therefore, in certain embodiments the yeast host cell is capable of conducting fermentation at low pH levels.

A suitable host cell may possess one or more favorable characteristics in addition to malate resistance, fumarate resistance, and/or low pH growth capability. For example, potential host cells exhibiting malate resistance may be further selected based on glycolytic rates, specific growth rates, thermotolerance, tolerance to biomass hydrolysate inhibitors, overall process robustness, and so on. These criteria may be evaluated prior to any genetic modification relating to a malate fermentation, fumarate fermentation, or reduction pathway, or they may be evaluated after one or more such modifications have taken place.

The level of gene expression and/or the number of exogenous genes to be utilized in a given cell will vary depending upon the identity of the host cell. For fully genome-sequenced yeasts, whole-genome stoichiometric models may be used to determine which enzymes should be expressed to develop a desired pathway malate or fumarate fermentation pathway. Whole-genome stoichiometric models are described in, for example, Hjersted Biotechnol Bioeng 97:1190 (2007) and Famili Proc Natl Acad Sci USA 100:13134 (2003).

For yeasts without a known genome sequence, sequences for genes of interest (either as overexpression candidates or as insertion sites) can typically be obtained using techniques known in the art. Routine experimental design can be employed to test expression of various genes and activity of various enzymes, including genes and enzymes that function in a malate fermentation, fumarate fermentation, or reduction pathway. Experiments may be conducted in which each enzyme is expressed in the yeast individually and in blocks of enzymes up to and including preferably all pathway enzymes, to establish which are needed (or desired) for improved malate and/or fumarate production. One illustrative experimental design tests expression of each individual enzyme as well as of each unique pair of enzymes, and further can test expression of all required enzymes, or each unique combination of enzymes. A number of approaches can be taken, as will be appreciated.

In certain embodiments, methods are provided for producing malate and/or fumarate from a genetically modified yeast cell as provided herein. In certain embodiments, these methods comprise providing a modified yeast cell as provided herein with at least one carbon source and culturing the yeast cell such that malate and/or fumarate is produced. The carbon source may be any carbon source that can be fermented by the yeast cell. Examples include, but are not limited to, twelve carbon sugars such as sucrose, hexose sugars such as glucose or fructose, glycan or other polymer of glucose, glucose oligomers such as maltose, maltotriose and isomaltotriose, panose, and fructose oligomers, and pentose sugars such as xylose, xylan, other oligomers of xylose, or arabinose. In certain embodiments, more than one type of genetically modified yeast cell may be present in the culture. Likewise, in certain embodiments one or more native yeast cells of the same or a different species than the genetically modified yeast cell may be present in the culture. In certain embodiments, culturing of the cells provided herein produces malate but little or no fumarate, or vice versa. In other embodiments, culturing of the cells produces a combination of malate and fumarate at various ratios.

In certain embodiments, culturing of the cells provided herein to produce malate or fumarate may be divided up into phases. For example, the cell culture process may be divided into a cultivation phase, a production phase, and a recovery phase. The following represent examples of specific conditions that may be used for each of these phases. One of ordinary skill in the art will recognize that these conditions may be varied based on factors such as the species of yeast being used, the desired yield, or other factors.

The medium will typically contain nutrients as required by the particular cell, including a source of nitrogen (such as amino acids, proteins, inorganic nitrogen sources such as ammonia or ammonium salts, and the like), and various vitamins, minerals and the like. In some embodiments, the cells of the invention can be cultured in a chemically defined medium. In one example, the medium is a DM medium containing around 5 g/L ammonium sulfate, around 3 g/L potassium dihydrogen phosphate, around 0.5 g/L magnesium sulfate, trace elements, vitamins and around 150 g/L glucose. The pH is adjusted may be allowed to range freely during cultivation, or may be buffered if necessary to prevent the pH from falling below or rising above predetermined levels. For example, the medium may be buffered to prevent the pH of the solution from falling below around 2.0 or rising above around 8.0 during cultivation. In certain of these embodiments, the medium may be buffered to prevent the pH of the solution from falling below around 3.0 or rising above around 7.0, and in certain of these embodiments the medium may be buffered to prevent the pH of the solution from falling below around 4.0 or rising above around 6.0. In certain embodiments, the fermentation medium is inoculated with sufficient yeast cells that are the subject of the evaluation to produce an OD₆₀₀ of 1.0. Unless explicitly noted otherwise, OD₆₀₀ as used herein refers to an optical density measured at a wavelength of 600 nm with a 1 cm pathlength using a model DU600 spectrophotometer (Beckman Coulter). The cultivation temperature may range from around 25-50° C., and the cultivation time may be up to around 120 hours. During cultivation, aeration and agitation conditions are selected to produce a desired oxygen uptake rate, such as for example around 2-25 mmol/L/hr. “Oxygen uptake rate” or OUR as used herein refers to the volumetric rate at which oxygen is consumed during the fermentation. Inlet and outlet oxygen concentrations can be measured with exhaust gas analysis, for example by mass spectrometers. OUR can be calculated by one of ordinary skill in the art using the Direct Method described in Bioreaction Engineering Principles 2^{nd} Edition, 2003, Kluwer Academic/Plenum Publishers, p. 449, equation 1.

In one example, the concentration of cells in the fermentation medium is typically in the range of about 0.1 to 20, preferably from 0.1 to 5, even more preferably from 1 to 3 g dry cells/liter of fermentation medium during the production phase. The fermentation may be conducted aerobically, microaerobically, or anaerobically, depending on pathway requirements. If desired, oxygen uptake rate can be varied throughout fermentation as a process control (see, e.g., WO03/102200). In certain embodiments, the modified yeast cells provided herein may perform especially well when cultivated under microaerobic conditions characterized by an oxygen uptake rate of from 2 to 25 mmol/L/hr. The medium may be buffered during the production phase such that the pH is maintained in a range of about 2.0 to about 8.0, about 3.0 to about 7.0, or about 4.0 to about 6.0. Suitable buffering agents are basic materials that neutralize the acid as it is formed, and include, for example, calcium hydroxide, calcium carbonate, sodium hydroxide, potassium hydroxide, potassium carbonate, sodium carbonate, ammonium carbonate, ammonia, ammonium hydroxide and the like. In general, those buffering agents that have been used in conventional fermentation processes are also suitable here.

In those embodiments where a buffered fermentation is utilized, acidic fermentation products may be neutralized to the corresponding salt as they are formed. In these embodiments, recovery of the acid involves regeneration of the free acid. This may be done by removing the cells and acidulating the fermentation broth with a strong acid such as sulfuric acid. This results in the formation of a salt by-product. For example, where a calcium salt is utilized as the neutralizing agent and sulfuric acid is utilized as the acidulating agent, gypsum is produced as a salt by-product. This by-product is separated from the broth, and the acid is recovered using techniques such as liquid-liquid extraction, distillation, absorption, and others (see, e.g., T. B. Vickroy, Vol. 3, Chapter 38 of Comprehensive Biotechnology, (ed. M. Moo-Young), Pergamon, Oxford, 1985; R. Datta, et al., FEMS Microbiol Rev, 1995, 16:221-231; U.S. Pat. Nos. 4,275,234, 4,771,001, 5,132,456, 5,420,304, 5,510,526, 5,641,406, and 5,831,122, and WO93/00440.

In other embodiments, the pH of the fermentation medium may be permitted to drop during cultivation from a starting pH that is at or above the lower pKa (3.40) of malate, typically 8 or higher, to at or below the lower pKa of the acid fermentation product, such as in the range of about 2.0 to about 3.4, in the range of from about 2.5 to about 3.4, or in the range from about 3.0 to about 3.4.

In still other embodiments, fermentation may be carried out to produce a product acid by adjusting the pH of the fermentation broth to at or below the lower pKa of the product acid prior to or at the start of the fermentation process. The pH may thereafter be maintained at or below the lower pKa of the product acid throughout the cultivation. In certain embodiments, the pH may be maintained at a range of about 2.0 to about 3.4, in the range of from about 2.5 to about 3.4, or in the range from about 3.0 to about 3.4.

In certain embodiments of the methods provided herein, the genetically modified yeast cells produce relatively low levels of ethanol. In certain embodiments, ethanol may be produced in a yield of 10% or less, preferably in a yield of 2% or less. In certain of these embodiments, ethanol is not detectably produced. In other embodiments, however, malate and/or fumarate and ethanol may be co-produced. In these embodiments, ethanol may be produced at a yield of greater than 10%, greater than 25%, or greater than 50%.

In certain embodiments of the methods provided herein, the final yield of malate or fumarate on the carbon source is at least 10%, at least 20%, at least 30%, at least 40%, at least 50%, or greater than 50% of the theoretical yield. In certain embodiments, the cells provided herein are capable of converting at least 80% or at least 90% by weight of a carbon source to malate or fumarate. The concentration, or titer, of malate or fumarate will be a function of the yield as well as the starting concentration of the carbon source. In certain embodiments, the titer may reach at least 1-3, at least 5, at least 10, at least 20, at least 30, at least 40, at least 50, or greater than 50 g/L at some point during the fermentation, and preferably at the end of the fermentation. In certain embodiments, the final yield of malate or fumarate may be increased by increasing the temperature of the fermentation medium, particularly during the production phase.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Selection of Host Yeast Cells Based on Malate Tolerance

A set of wild-type yeast strains were tested for their ability to grow in the presence of malate.

The range of malate concentrations to utilize in primary screening procedures was determined by evaluating the ability of seven wild-type yeast strains (Candida sonorensis, Candida zemplinina, I. orientalis strain PTA-6658, I. orientalis strain CD1822, Kluyveromyces lactis, K. marxianus, S. cerevisiae strain CENPK 113-7D) to grow on media containing varying levels of malate. Cells were streaked onto YPD plates and grown overnight. A cell slurry with an OD₆₀₀ of around 4 was made in YPD media, pH 3.0, and this slurry was used to inoculate microtiter wells containing various concentrations of malate to an OD₆₀₀ of 0.05. Plates were covered with a gas permeable membrane and incubated in a 30° C./300 rpm shaker overnight. The optical densities of each well were measured at a wavelength of 600 nm in a GENios model plate reader (Tecan), and plates were observed visually for growth. The highest malate concentration that one or more of the strains grew in (350 g/L) was chosen as the upper range for primary screening procedure.

For the primary screening procedure, 91 wild-type yeast strains were screened for growth on microtiter plates at pH 2.85 with 0 g/L, 150 g/L, 300 g/L, or 350 g/L pH 2.85 malate using the same protocol used for range finding. A fresh YPD plate was used for each strain, and a slurry with an OD₆₀₀ of around 4 was made in YPD media, pH 3.0. The slurry was used to inoculate each well to an OD₆₀₀ of 0.05. Plates were covered with a gas permeable membrane, and incubated in a 30° C./300 rpm shaker overnight. Optical densities of each well were measured at 600 nm in a GENios model plate reader, and plates were observed visually for growth. To account for plate-to-plate variability, I. orientalis PTA-6658 was included on all plates as a control. This control strain exhibited significant variability across the plates, so the original primary screen was deemed insufficient to identify the best strains for advancement to the secondary screen. As such, an additional screen was added wherein the 24 strains that grew as well or better than the I. orientalis control strain were re-tested. The protocol for this additional screen was the same as that described above, except the malate concentrations were 200 g/L, 300 g/L, and 400 g/L. The 300 g/L and 400 g/L concentrations proved too harsh for effective strain differentiation, so only the 200 g/L concentration was used to identify resistant strains. Ten strains showed the ability to grow at this concentration to an optical density greater than or equal to 50% of the optical density achieved with the same strain in 0 g/L malic acid. These ten strains were advanced to the secondary screens. It is expected that strains not advancing to the secondary screen would exhibit economically inferior performance in a commercial fermentation process. However, it is possible that one or more such strains could nonetheless meet the minimum requirements for a commercially viable fermentation process.

For the first secondary screen, growth rates were measured in YPD media containing 0 g/L malate at pH 3.0 or 200 g/L malate at pH 2.85. Shake flasks were inoculated with biomass harvested from seed flasks grown overnight to an OD₆₀₀ of 6 to 10. 250 mL baffled growth rate flasks (50 mL working volume) were inoculated to an OD₆₀₀ of 0.1 and grown at 250 rpm and 30° C. Samples were taken throughout the time course of the assay and analyzed for biomass growth via OD₆₀₀. The resulting OD₆₀₀ data was plotted and growth rates were established. Results are summarized in Table 1.

TABLE 1
Growth rate in malate:

| | 0 g/L malate | 200 g/L malate |
|---|---|---|
| | (pH 3.0) | (pH 2.85) |
| Strain | (h⁻¹) | (h⁻¹) |
| Issatchenkia orientalis | 0.71 | 0.44 |
| ATCC PTA-6658 | | |
| Issatchenkia orientalis | 0.69 | 0.42 |
| CD1822 | | |
| (Cargill collection) | | |
| Candida sorbosivorans | 0.26 | 0.34 |
| CD1978 | | |
| (Cargill collection) | | |
| Candida vanderwaltii | 0.47 | 0.39 |
| MUCL 30000 | | |
| Issatchenkia orientalis | 0.73 | 0.36 |
| ATCC 60585 | | |
| Candida guilliermondii | 0.44 | 0.42 |
| ATCC 20118 | | |
| Candida lambica | 0.81 | 0.33 |
| ATCC 38617 | | |
| Candida sorbosivorans | 0.45 | 0.37 |
| ATCC 38619 | | |
| Issatchenkia orientalis | 0.74 | 0.39 |
| ATCC 24210 | | |
| Saccharomyces bulderi | 0.41 | 0.39 |
| ATCC MYA-404 | | |

For the second secondary screen, glucose consumption was measured in YPD media containing 0 g/L malate at pH 3.0 or 200 g/L malate at pH 2.85. Shake flasks were inoculated with biomass harvested from seed flasks grown overnight to an OD₆₀₀ of 6 to 10. 250 mL baffled glycolytic assay flasks (50 mL working volume) were inoculated to an OD₆₀₀ of 0.1 and grown at 250 RPM and 30° C. Samples were taken throughout the time course of the assay and analyzed for glucose consumption using a 2700 Biochemistry Analyzer (Yellow Springs Instruments, YSI). The resulting data was plotted and glucose consumption rates were established. Results are summarized in Table 2.

TABLE 2
Glucose consumption rate in malate:

| | 0 g/L malate | 200 g/L malate |
|---|---|---|
| | (pH 3.0) | (pH 2.85) |
| Strain | (g L⁻¹ h⁻¹) | (g L⁻¹ h⁻¹) |
| Issatchenkia orientalis | 4.2 | 3.3 |
| ATCC PTA-6658 | | |
| Issatchenkia orientalis | 4.2 | 3.3 |
| CD1822 | | |
| Candida sorbosivorans | >2.6 | 1.85 |
| CD1978 | | |
| Candida vanderwaltii | 0.4 | 1.3 |
| MUCL 30000 | (not finished) | |
| Issatchenkia orientalis | 4.2 | 2.7 |
| ATCC 60585 | | |
| Candida guilliermondii | 3.1 | 1.9 |
| ATCC 20118 | | |
| Candida lambica | 4.2 | 3.2 |
| ATCC 38617 | | |
| Candida sorbosivorans | 3.4 | 1.4 |
| ATCC 38619 | | |
| Issatchenkia orientalis | 4.2 | 3.1 |
| ATCC 24210 | | |
| Saccharomyces bulderi | 5.3 | 3.15 |
| ATCC MYA-404 | | |

To identify the most attractive candidates for malate production, strain performance was graded in three categories. Two of these categories were based on different aspects of growth rate: 1) growth rate at highest acid concentration and 2) slope of the growth rates plotted against acid concentration. The third category was the glycolytic rate at the highest acid concentration. Grading was done on a normalized scale using the highest and lowest value for each rating as the normalized boundaries. Each strain thus received a grade of 0 to 1 for each category, with 1 being the highest possible score. The overall rating of a strain was the sum of the normalized value for the three categories. A weighted score was made in which the growth rate and glycolytic rate were equally weighted. In this case the glycolytic rate at the highest acid concentration was weighted at 50%, while the two growth rate ratings were weighted at 25% each. In accordance with the description above, the final score for each strain was calculated as follows:

Final score for strain X=(actual growth rate in highest acid concentration−slowest growth rate)*0.25 (fastest growth rate−slowest growth rate)+

(slope of growth rates of strain X−lowest growth rate slope)*0.25 (largest growth rate slope−lowest growth rate slope)+

(actual glycolytic rate in highest acid concentration−slowest glycolytic rate)*0.50 (fastest glycolytic rate−slowest glycolytic rate)

Normalized values for each category and the final weighted score for each strain are summarized in Table 3.

TABLE 3
Normalized strain grades in malate:

| | | | Glucose | |
|---|---|---|---|---|
| | Growth | | consumption | |
| | rate | | rate | |
| | 200 g/L | Growth | 200 g/L | |
| | malate | rate | malate | Weighted |
| Strain | (pH 2.85) | slope | (pH 2.85) | score |
| Issatchenkia orientalis | 1.00 | 0.67 | 1.00 | 0.92 |
| ATCC PTA-6658 | | | | |
| Issatchenkia orientalis | 0.82 | 0.67 | 1.00 | 0.87 |
| CD1822 | | | | |
| Saccharomyces bulderi | 0.55 | 1.00 | 0.93 | 0.85 |
| ATCC MYA-404 | | | | |
| Issatchenkia orientalis | 0.55 | 0.56 | 0.90 | 0.73 |
| ATCC 24210 | | | | |
| Candida guilliermondii | 0.82 | 1.00 | 0.30 | 0.61 |
| ATCC 38617 | | | | |
| Candida lambica | 0.00 | 0.41 | 0.95 | 0.58 |
| ATCC 38617 | | | | |
| Issatchenkia orientalis | 0.27 | 0.54 | 0.70 | 0.55 |
| ATCC 60585 | | | | |
| Candida vanderwaltii | 0.55 | 0.92 | 0.00 | 0.37 |
| MUCL 30000 | | | | |
| Candida sorbosivorans | 0.36 | 0.92 | 0.05 | 0.35 |
| ATCC 24120 | | | | |
| Candida sorbosivorans | 0.09 | 0.00 | 0.28 | 0.16 |
| CD1978 | | | | |

The same procedures were utilized to screen, rate, and score the original 91 wild-type yeast strains from the primary screen with media containing 0, 30, 45, and 60 g/L lactic acid at pH 3.0 (˜80% free acid). Due to difficulties in properly scoring some very weak growth that occasionally occurred at 60 g/L, 20 strains were re-tested in the primary screen. Of these 20 strains, eight were eliminated due to very slow growth relative to the rest of the test group. The remaining 12 strains were advanced into secondary screening. The secondary screen for lactic acid was the same as that described above for malate, except for changes in concentrations and pH values. Growth rates were measured at 0 g/L lactic acid, pH 3.0 and 50 g/L lactic acid, pH 2.85, and glucose consumption rates were measured in the presence of 50 g/L lactic acid, pH 2.85. Normalized values and weighted and summed scores were calculated for each strain as described above for malate. These results are summarized in Table 4.

TABLE 4
Normalized strain grades in lactic acid:

| | Growth rate | Growth | | |
|---|---|---|---|---|
| | 50 g/L | rate | Glycolic | Weighted |
| Strain | lactic acid | slope | rate | score |
| Candida lambica | 0.92 | 1 | 1 | 0.98 |
| ATCC 38617 | | | | |
| Issatchenkia orientalis | 0.94 | 0.95 | 1 | 0.97 |
| ATCC PTA-6658 | | | | |
| Issatchenkia orientalis | 1.00 | 0.86 | 1 | 0.97 |
| CD1822 | | | | |
| Issatchenkia orientalis | 0.89 | 0.73 | 1 | 0.91 |
| ATCC 24210 | | | | |
| Candida zemplinina | 0.22 | 0.95 | 1 | 0.79 |
| Saccharomyces bulderi | 0.47 | 0.45 | 1 | 0.73 |
| ATCC MYA-404 | | | | |
| Saccharomyces bayanus | 0.08 | 0.91 | 0.96 | 0.73 |
| Saccharomyces bulderi | 0.5 | 0.23 | 1 | 0.68 |
| ATCC MYA-402 | | | | |
| Candida milleri | 0 | 0.64 | 0.92 | 0.62 |
| ATCC 60592 | | | | |
| Candida sorosivorans | 0.28 | 0.95 | 0.59 | 0.60 |
| Kodamaea ohmeri | 0.42 | 0 | 0.76 | 0.49 |
| Candida geochares | 0.17 | 0.27 | 0.69 | 0.46 |
| Saccharomyces javensis | 0.11 | 0.68 | 0 | 0.20 |

Based on these weighted scores, the strains were rank ordered. These rank orders are summarized in Table 5.

TABLE 5
Rank order of strains in lactic acid and malate:

| | Lactic acid | Malate |
|---|---|---|
| Strain | rank order | rank order |
| Candida lambica | 1 | 6 |
| ATCC 38617 | | |
| Issatchenkia orientalis | 2 | 1 |
| ATCC PTA-6658 | | |
| Issatchenkia orientalis | 3 | 2 |
| CD1822 | | |
| Issatchenkia orientalis | 4 | 4 |
| ATCC 24210 | | |
| Candida zemplinina | 5 | NA |
| PYCC 04-501 | | |
| Saccharomyces bulderi | 6 | 3 |
| ATCC MYA-404 | | |
| Saccharomyces bayanus | 7 | NA |
| ATCC 90739 | | |
| Saccharomyces bulderi | 8 | NA |
| ATCC MYA-402 | | |
| Candida milleri | 9 | NA |
| ATCC 60592 | | |
| Candida sorbosivorans | 10 | 9 |
| ATCC 38619 | | |
| Kodamaea ohmeri | 11 | NA |
| ATCC 20282 | | |
| Candida sorbosivorans | 12 | 10 |
| CD1978 | | |
| Candida guilliermondii | NA | 5 |
| ATCC 20118 | | |
| Issatchenkia orientalis | NA | 7 |
| ATCC 60585 | | |
| C. vanderwaltii | NA | 8 |
| MUCL 30000 | | |

Three different isolates from the species I. orientalis were in the top four strains for both organic acids, indicating that this species is a promising candidate for organic acid production. However, beyond I. orientalis, the strain ranks diverged significantly for the two different acids. The top strain for lactic acid was ranked only sixth for malic acid, and five of the twelve strains promoted to the secondary screen for lactic acid were not promoted to the secondary screen for malate. Therefore, tolerance to lactic acid was generally shown to be a poor predictor of tolerance to malic acid, meaning that ideal strains for malate production cannot be identified based on tolerance to lactic acid. This is further highlighted by comparing the strains that showed malate resistance above with the list of eight strains identified as preferred hosts for organic acid production in WO03/049525. While two of these strains (C. diddensiae and C. entomophila) could not be obtained for testing, the other six (C. sonorensis, C. methanosorbosa, C. parapsilosis, C. naeodendra, C. krusei, and C. blankii) were included in the primary screen described above. Of these six, only C. krusei (tested as I. orientalis) demonstrated sufficient malic acid tolerance to warrant promotion to the secondary screen for malic acid.

### Example 2

### Mutagenesis and Selection of Mutant Strains Having Malate Resistance

Yeast cells selected in Example 1 are subjected to mutagenesis and exposed to selection pressure in order to identify mutants with high malate tolerance.

For example, yeast cells from a fresh YP (yeast extract/peptone)+20 g/L glucose plate or liquid culture (OD₆₀₀ 1-4) are resuspended in sterile water to an OD₆₀₀ of around 10. 200 μL aliquots of this cell suspension are pipetted into individual tubes and exposed to 3 μL ethane methyl sulfonate (EMS) for approximately one hour, which kills around 65% of the cells. Higher EMS concentrations can also be used to increase the kill rate. After exposure, cells are neutralized with 5% sodium thiosulfate, washed in PBS buffer, recovered in rich media for approximately four hours, and cultured on selective media. Mock samples (no EMS) are also run to ensure that the conditions are selective. Alternatively, cell can be mutagenized using UV irradiation.

To select for malate resistant mutant strains, aliquots of the EMS-treated cell suspension (approximately 2×10⁸ of mutagenized cells) are plated onto a potato dextrose agar (PDA) or another media containing malate at a level at which the parental strain does not grow or grows very slowly. These plates are incubated for several days until colonies appear. Single colonies are purified, streaked on non-selective media to eliminate any adaptive effects of the selection, and re-tested on selective media to confirm increased resistance. Resistant strains are then tested in a shake flask format with periodic sampling for HPLC analysis of products and substrates. Alternatively, selection for malate tolerance may be done by chemostat or serial shake flask evolution. Additional rounds of mutagenesis and selection can be performed. Mutagenesis can be used to increase the resistance of a host that does not natively meet malate production requirements so that it has the necessary attributes for commercial malate production.

### Example 3

### Deletion of Both Alleles of CYB2A, GPD1, and CYB2B from I. orientalis Strain CD1822

Both alleles of CYB2A, GPD1, and CYB2B are deleted from I. orientalis strain CD1822. As discussed above, CD1822 is an evolved lactic acid resistant strain isolated from a chemostat that also exhibited a high degree of malate tolerance.

### Example 3A

### Deletion of Both CYB2A Alleles from I. orientalis Strain CD1822

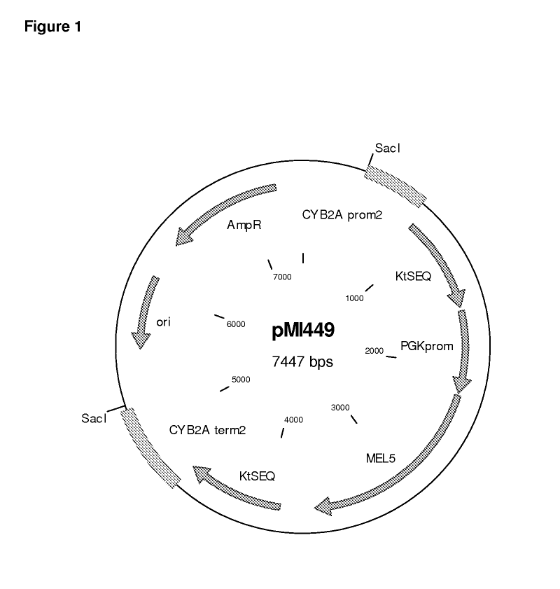

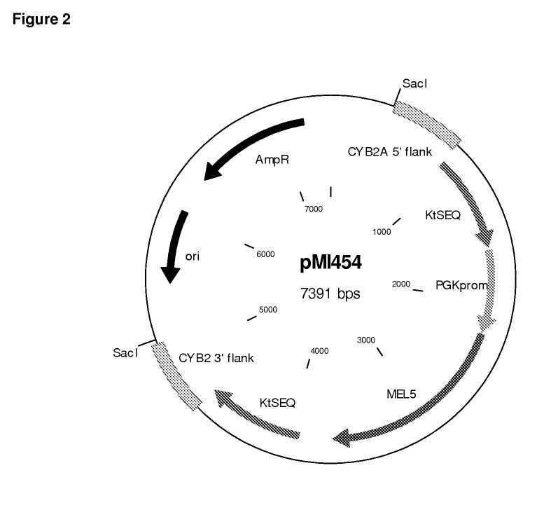

Plasmids pMI449 (FIG. 1) and pMI454 (FIG. 2) are used to delete both copies of the L-lactate:ferricytochrome c oxidoreductase (CYB2A) gene (SEQ ID NO:41) in I. orientalis strain CD1822, a lactic acid resistant strain of I. orientalis isolated from the environment. pMI449 and pMI454 were both described in WO07/106,524. Each plasmid contains 5′ and 3′ flanking regions from I. orientalis CYB2A separated by a selection marker cassette comprising the S. cerevisiae MEL5 gene operatively linked to a PGK promoter. This selection marker cassette is flanked on either end by a sequence (“KtSEQ”) from K. thermotolerans. The 5′ and 3′ CYB2A flanking regions in pMI449 correspond to nucleotides from 913 to 487 bp upstream of the start of the predicted ORF and nucleotides from 90 to 676 bp downstream of the stop codon of the predicted ORF, respectively. The 5′ and 3′ CYB2A flanking regions in pMI454 correspond to nucleotides from 466 to 7 bp upstream of the predicted ORF and nucleotides from 402 bp upstream to 77 bp downstream of the predicted stop codon, respectively.

The first CYB2A allele is deleted by transforming strain CD1822 with pMI449 digested with SacI using lithium acetate transformation (Gietz Met Enzymol 350:87 (2002)). Transformants are selected on yeast nitrogen base (YNB)+2% melibiose plate containing x-α-gal (5-bromo-4-chloro-3-indolyl-α-D-galactoside). Blue-colored transformants are visible after around 4 days of growth at 30° C. Transformants are picked and plated for single colonies on Yeast Extract/Peptone/20 g/L glucose plates (YPD) containing x-α-gal. A single blue colony for each transformant is picked and re-streaked to YPD plates. Genomic DNA is isolated from the purified transformants, and replacement of the CYB2A gene is confirmed by PCR. To obtain strains where the MEL5 marker has undergone spontaneous recombination to excise it from the chromosome, the transformant is grown for several rounds in liquid YPD (100 g/L glucose) at 250 rpm and 30° C. A dilution series is plated onto YPD plates overlaid with x-α-gal, and grown overnight at 30° C. A white colony (indicative of the loop-out of the MEL5 marker cassette) is selected and re-streaked to YPD+x-α-gal plates. A white colony is selected and genomic DNA is prepared. Disruption of one allele of the native CYB2A gene is verified by PCR using primers oMM173 (SEQ ID NO:50) and oTM123 (SEQ ID NO:54).

The second CYB2A allele is deleted from this transformant by transforming with pMI454 digested with SacI. Transformants are obtained and purified as described above and analyzed by PCR for the absence of a 1000 bp CYB2A-specific PCR product using primers oMM175 (SEQ ID NO:52) and oMM176 (SEQ ID NO:53). The MEL5 marker derived from plasmid pMI454 is looped out of a transformant having a deletion of both CYB2A alleles via recombination as before, and confirmed by PCR using primers oMM172 (SEQ ID NO:49) and oMM173 (SEQ ID NO:50). This transformant is designated strain 2610.

### Example 3B

### Deletion of Both GPD1 Alleles from I. orientalis Strain 2610

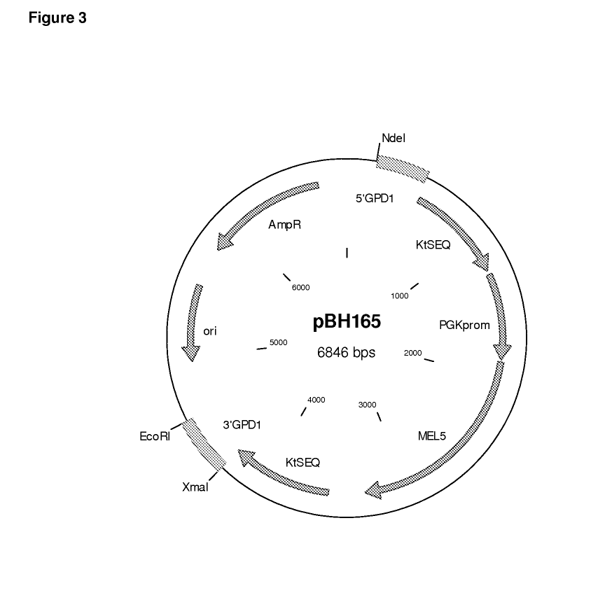

Plasmid pBH165 (FIG. 3) is used to delete one allele of the GPD1 gene (SEQ ID NO:35) from I. orientalis strain 2610. pBH165, which was described in WO07/106,524, contains upstream and downstream fragments of the I. orientalis GPD1 gene separated by a selection marker cassette comprising S. cerevisiae MEL5 operatively linked to a PGK promoter and surrounded by KtSEQ flanking sequences. The upstream and downstream fragments of the GPD1 gene correspond to nucleotides from 1 to 302 bp and from 322 to 608 bp downstream of the start codon, respectively.

Strain 2610 is transformed with pBH165 digested with NdeI and EcoRI using lithium acetate transformation, and transformants are selected on YNB+2% melibiose plate overlaid with x-α-gal. Blue-colored transformants are visible after around 4 to 7 days of growth at 30° C. Transformants are picked and plated for single colonies on YPD plates containing x-α-gal, and a single transformant is picked and re-streaked to YPD plates. Genomic DNA is isolated from the transformants, and disruption of one allele of the GPD1 gene is confirmed by one or more PCR reactions. The resulting transformant is designated strain 2639.

To obtain strains where the MEL5 marker has undergone spontaneous recombination to excise it from the chromosome, strain 2639 is grown for several rounds in liquid YPD (100 g/L glucose) at 250 rpm and 30° C. A dilution series is plated onto YPD plates overlaid with x-α-gal, and grown overnight at 30° C. A white colony (indicative of the loop-out of the MEL5 marker cassette) is selected and re-streaked to YPD+x-α-gal plates. A white colony is selected and streaked onto a YPD plate. Disruption of one allele of the native GPD1 gene and loss of the MEL5 marker is verified by PCR. The resultant transformant is designated strain 2643.

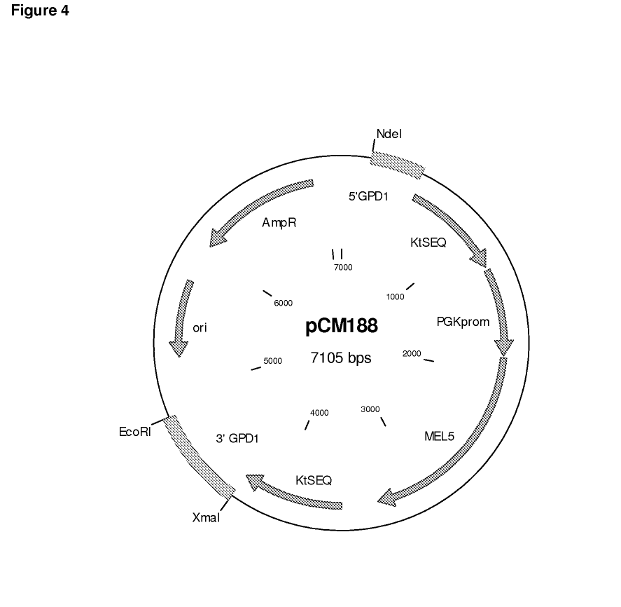

Plasmid pCM188 (FIG. 4) is used to delete the second GPD1 allele from I. orientalis strain 2643. pCM188 was generated by amplifying a 3′ flanking region (corresponding to nucleotides from 1169 to 1770 bp downstream of the GPD1 gene start codon) of the GPD1 gene using primers CMO588 (SEQ ID NO:57) and CMO589 (SEQ ID NO:58). The primers included nucleotides for incorporating an XmaI site at the 5′ end and an EcoRI site at the 3′ end of the amplified DNA. The resultant product is digested with XmaI and EcoRI and ligated to similarly digested pBH165 to generate pCM188, which contains upstream and downstream fragments of the I. orientalis GPD1 gene separated by a selection marker cassette comprising S. cerevisiae MEL5 operatively linked to a PGK promoter and surrounded by KtSEQ flanking sequences.

Strain 2643 is transformed with pCM188 digested with NdeI and EcoRI using lithium acetate transformation, and transformants are selected on YNB+2% melibiose plate overlaid with x-α-gal. Blue-colored transformants are visible after around 4 to 7 days of growth at 30° C. Transformants are picked and plated for single colonies on YPD plates containing x-α-gal, and a single transformant is picked and re-streaked to YPD plates. Genomic DNA is isolated from the transformants, and disruption of the second GPD1 allele is confirmed by one or more PCR reactions. The resulting transformant is designated strain 2644.

To obtain strains where the MEL5 marker has undergone spontaneous recombination to excise it from the chromosome, strain 2644 is grown for several rounds in liquid YPD (100 g/L glucose) at 250 rpm and 30° C. A dilution series is plated onto YPD plates overlaid with x-α-gal, and grown overnight at 30° C. A white colony (indicative of the loop-out of the MEL5 marker cassette) is selected and re-streaked to YPD+x-α-gal plates. A white colony is selected and streaked onto a YPD plate. Disruption of the second allele of the native GPD1 gene and loss of the MEL5 marker is verified by PCR. The resultant transformant is designated strain 2652.

### Example 3C

### Deletion of Both CYB2B Alleles from I. orientalis Strain 2652

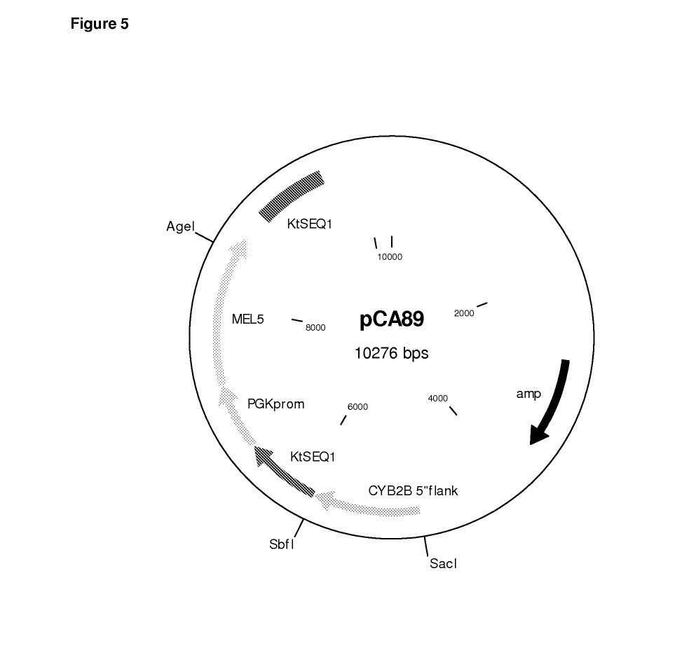

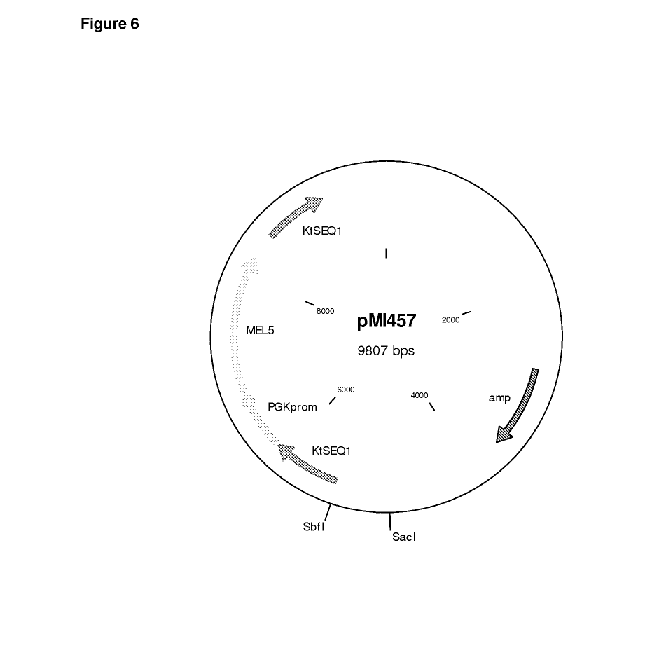

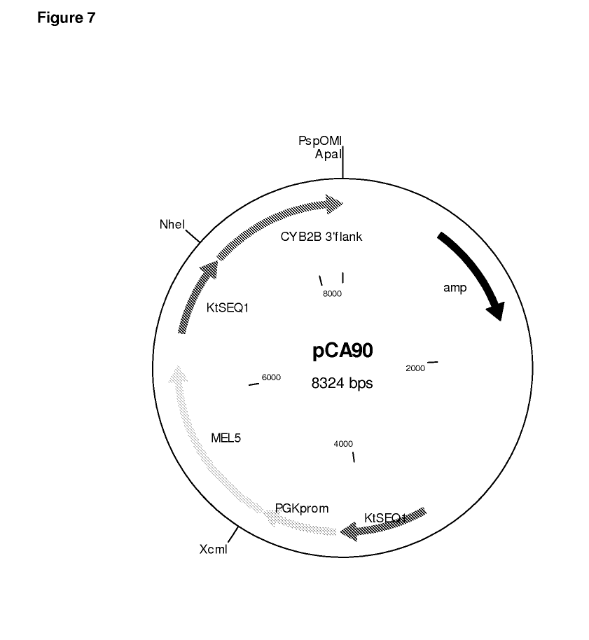

Plasmids pCA89 and pCA90 are used to delete one allele of the CYB2B gene (SEQ ID NO:43) from I. orientalis strain 2652. pCA89 (FIG. 5) is generated by amplifying a 5′ flanking region of CYB2B using PCR primers oCA385 (SEQ ID NO:59) and oCA386 (SEQ ID NO:60), which incorporates SacI and SbfI restriction sites into the PCR product, then digesting the product and ligating to pMI457 cut with the same enzymes. pMI457 (FIG. 6) contains a MEL5 gene operably linked to a PGK promoter and surrounded by KtSEQ flanking sequences. pCA90 (FIG. 7) is generated by amplifying a 3′ flanking region of CYB2B using PCR primers oCA387 (SEQ ID NO:61) and oCA388 (SEQ ID NO:62), which incorporates NheI and PspOMI restriction sites into the PCR product, then digesting the product and ligating to pMI457 cut with the same enzymes.

Strain 2652 is transformed with pCA89 digested with SacI and AgeI and pCA90 digested with XcmI and ApaI using lithium acetate transformation. The two plasmid fragments are able to recombine during transformation to form a functional MEL5 gene. Transformants are selected on YNB+2% melibiose plate overlaid with x-α-gal. Blue-colored transformants are visible after around 4 to 7 days of growth at 30° C. Transformants are picked and plated for single colonies on YPD plates containing x-α-gal, and a single transformant is picked and re-streaked to YPD plates. Genomic DNA is isolated from the transformants, and replacement of a first CYB2B allele is confirmed by one or more PCR reactions. The resulting transformant is designated strain 2719.

To obtain strains where the MEL5 marker has undergone spontaneous recombination to excise it from the chromosome, strain 2719 is grown for several rounds in liquid YPD (100 g/L glucose) at 250 rpm and 30° C. A dilution series is plated onto YPD plates overlaid with x-α-gal, and grown overnight at 30° C. A white colony (indicative of the loop-out of the MEL5 marker cassette) is selected and re-streaked to YPD+x-α-gal plates. A white colony is selected and streaked onto a YPD plate. Disruption of a first allele of the native CYB2B gene and loss of the MEL5 marker is verified by PCR. The resultant transformant is designated strain 2721.

The second CYB2B allele is deleted from strain 2721 bp transforming with the same pCA89 and pCA90 fragments used to transform strain 2652. Transformants are obtained and purified as described above, and strains from which the MEL5 marker has been excised by recombination are generated using the methods described above. Disruption of both CYB2B alleles and loss of the MEL5 gene is verified by PCR. The resultant transformant is designated strain 2732.

The various CYB2A, GPD1, and CYB2B deletion strains generated in Example 3 are summarized in Table 6.

TABLE 6
I. orientalis CYB2A, GPD1, and CYB2B deletion strains:

| Strain | | Parent |
|---|---|---|
| name | Description | strain |
| CD1822 | Lactic acid-resistant | — |
| | parent strain | |
| 2610 | CYB2A deletion (2) | |
| 2639/2643 | CYB2A deletion (2) | 2610 |
| | GPD1 deletion (1) | |
| 2644/2652 | CYB2A deletion (2) | 2643 |
| | GPD1 deletion (2) | |
| 2719/2721 | CYB2A deletion (2) | 2652 |
| | GPD1 deletion (2) | |
| | CYB2B deletion (1) | |
| 2732 | CYB2A deletion (2) | 2721 |
| | GPD1 deletion (2) | |
| | CYB2B deletion (2) | |

### Example 4

### Construction of cre Expression Plasmids DVB10 and pVB32

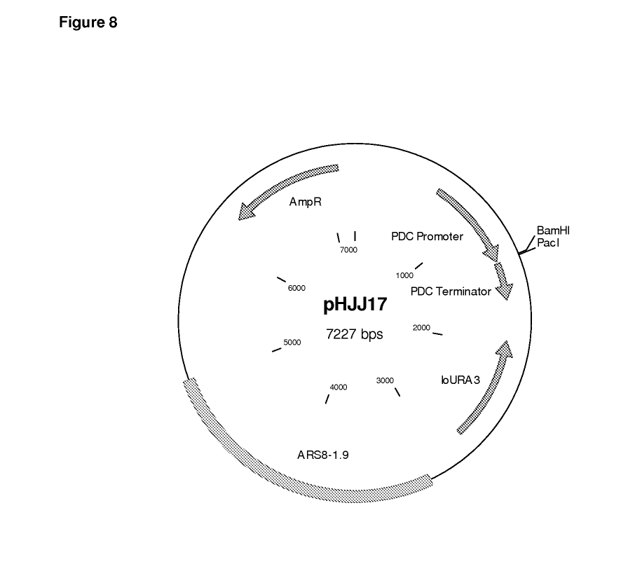

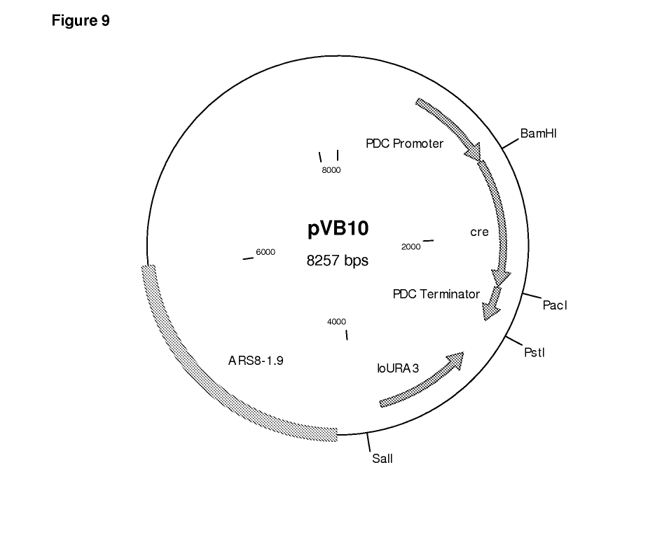

The cre recombinase gene is synthesized using the native cre protein sequence as a reference. This gene is PCR amplified from template DNA (Blue Heron Biotechnologies) representing a codon-optimized version of the bacteriophage P1 CRE gene (SEQ ID NO:47, encoding polypeptide of SEQ ID NO:48) using PCR primers oVB5 (SEQ ID NO:64) and oVB6 (SEQ ID NO:65) and cloned into pCR2.1-TOPO (Invitrogen) to produce pVB15a. pVB15a is digested with BamHI and PacI to generate a 1 kb cre fragment, and this fragment is ligated into similarly digested pHJJ17 (FIG. 8). The resultant vector, pVB10 (FIG. 9), contains the cre gene operatively linked to a PDC promoter and terminator. The vector also contains a URA3 selection marker gene from I. orientalis.

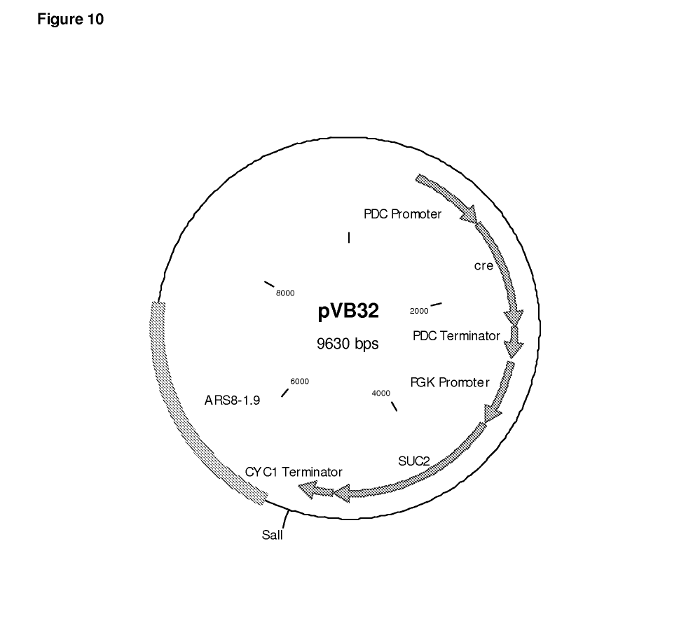

To replace the URA3 selection marker in pVB10 with the SUC2 selection marker, a SUC2 expression cassette was amplified from pTMC82. This SUC2 expression cassette contains the S. cerevisiae SUC2 gene operatively linked to the I. orientalis PGK1 promoter and the S. cerevisiae CYC1 terminator. Amplification was performed using primers oTM298 (SEQ ID NO:55) and oTM299 (SEQ ID NO:56), which add NsiI and SalI restriction sites to the product. The PCR product was digested with NsiI and SalI and ligated to pVB10 digested with SalI and PstI (NsiI and PstI have compatible cohesive ends) to produce pVB32 (FIG. 10).

### Example 5

### Insertion of PYC1 at the PDC1 Locus in I. orientalis Strain 2732

A PYC1 expression cassette is inserted at one or both PDC1 alleles in I. orientalis strain 2732 (Example 3).

### Example 5A

### Construction of PDC1 Deletion Construct PKWB21

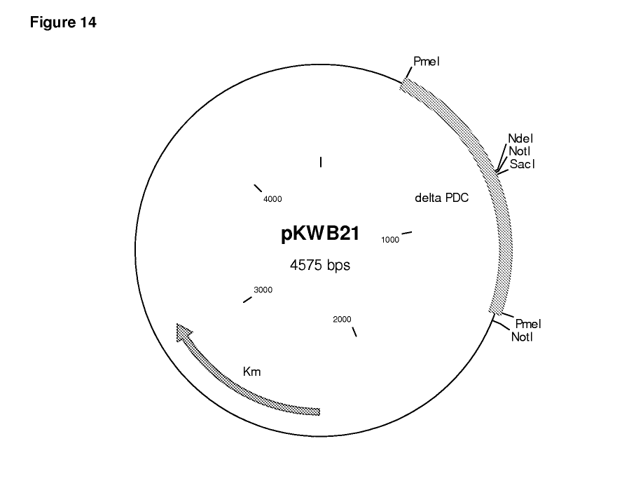

Upstream and downstream regions of the PDC1 gene (SEQ ID NO:31) were amplified in order to generate a PDC1 deletion construct. The upstream and downstream regions correspond to nucleotides from 496 bp upstream to the start codon of PDC1 and from the stop codon to 539 bp downstream, respectively. Amplification of the upstream region is performed using primers oKW70 (forward, SEQ ID NO:81) and oKW71 (reverse, SEQ ID NO:82), which adds a PmeI restriction site and NdeI, NotI, and SacI restriction sites, respectively, to the product. Amplification of the downstream region is performed using primers oKW72 (forward, SEQ ID NO:83) and oKW73 (reverse, SEQ ID NO:84), which adds NdeI, NotI, and SacI restriction sites and a PmeI restriction site, respectively, to the product. The two fragments are amplified independently, then assembled into a full-length product with a 2-stage PCR protocol. The first stage uses 10 cycles (98° C. 10 s, 55° C. 20 s, 72° C. 60 s) with no primers, and the second stage uses 20 cycles (98° C. 10 s, 55° C. 20 s, 72° C. 60 s) with upstream forward and downstream reverse primers. The full-length product is gel purified, cloned into pCR-BluntII (Invitrogen), and sequenced. The plasmid confirmed to have correct sequence is subjected to quickchange PCR using Phusion polymerase to eliminate the plasmid borne SacI site. Correct plasmids are confirmed by digestion with SacI and sequencing. The final PDC1 deletion construct is designated pKWB21 (FIG. 14).

### Example 5B

### Construction of I. orientalis PYC1 Expression Constructs pKF043 and pKF045

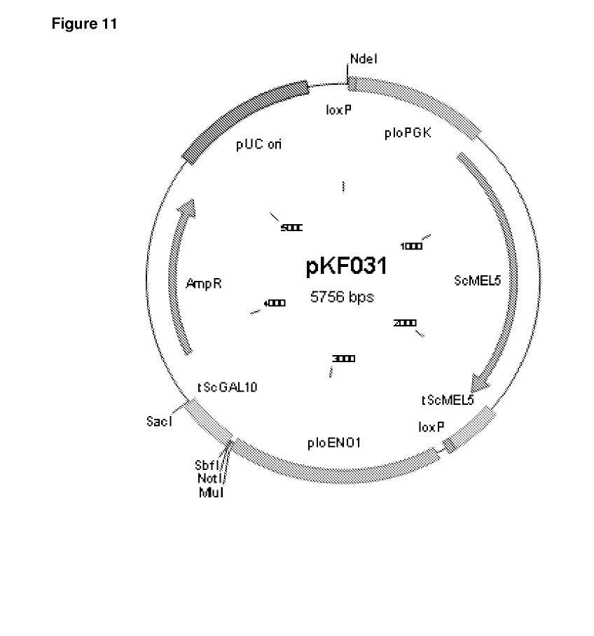

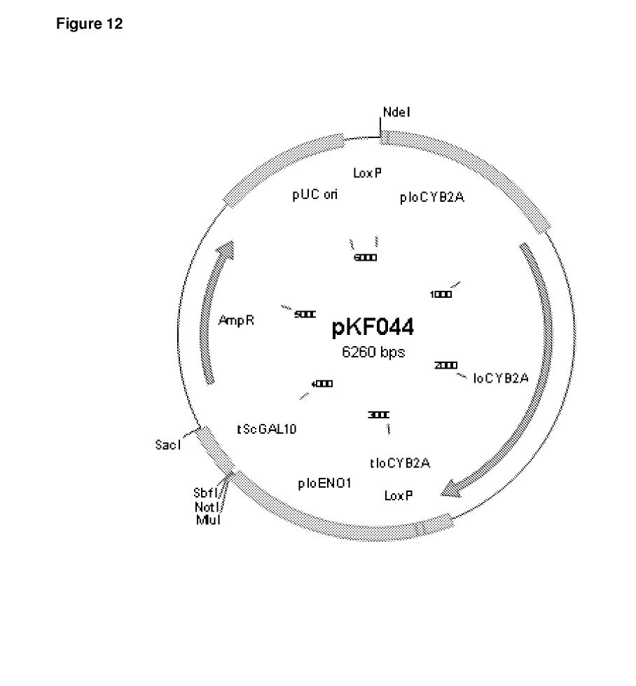

The PYC1 gene from I. orientalis (SEQ ID NO:7) is amplified from genomic DNA using Phusion polymerase and primers oKF245 (SEQ ID NO:117) and oKF246 (SEQ ID NO:118), which contain an MluI site and an SbfI site, respectively. After amplification, the product is gel purified, digested with MluI and SbfI, and ligated to similarly digested pKF031 and pKF044. pKF031 (FIG. 11) and pKF044 (FIG. 12) are constructed from pUC19 backbones, and both contain a multiple cloning site containing MluI, NotI, and SbfI sites operatively linked to the I. orientalis ENO promoter and the S. cerevisiae GAL10 terminator. pKF031 also contains a selection marker cassette comprising the S. cerevisiae MEL5 gene operatively linked to the I. orientalis PGK promoter. This selection marker cassette is flanked by loxP sites. pKF044 contains an expression cassette comprising the I. orientalis CYB2A promoter, gene, and terminator. This expression cassette is flanked by loxP sites.

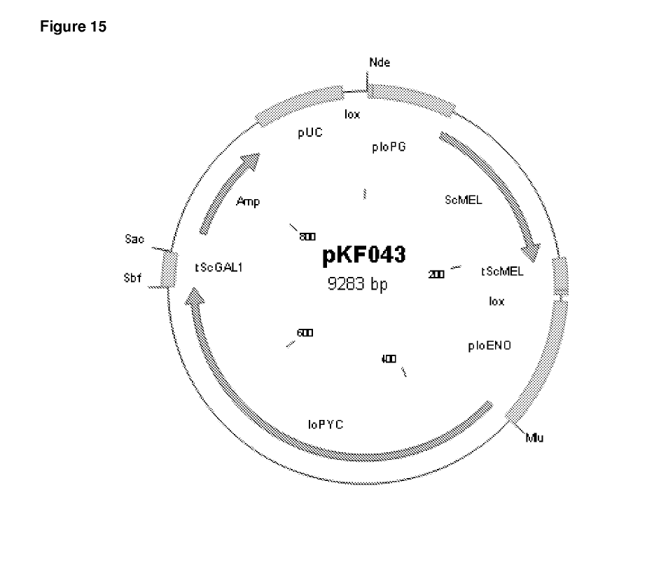

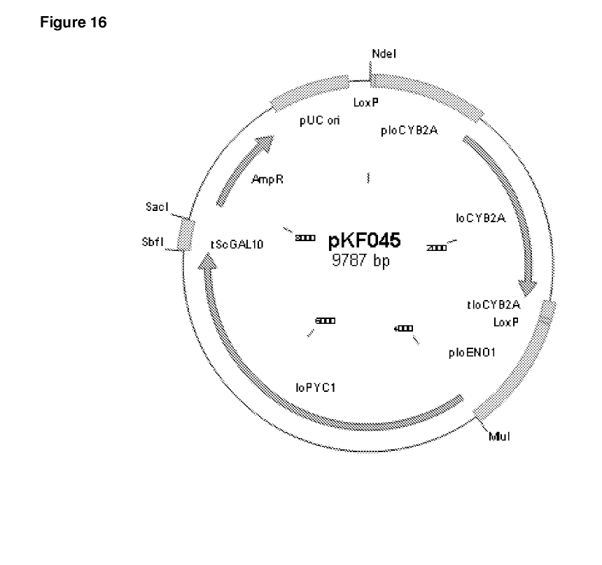

The plasmids are transformed into E. coli, and transformants are selected on LB plates containing 100 μg/ml carbenicillin and screened using primers flanking the NotI site of pKF031 and pKF044 (oKW93 (SEQ ID NO:95) and oKW95 (SEQ ID NO:96)). Quickchange PCR is performed using primers oKW96 (SEQ ID NO:97) and oKW97 (SEQ ID NO:98) to eliminate an internal NdeI site (T2847C). Correct plasmids are confirmed by sequencing, and the final constructs are designated pKF043 (MEL5 marker) (FIG. 15) and pKF045 (CYB2A marker) (FIG. 16).

### Example 5C

### Construction of S. cerevisiae PYC1 Expression Constructs pKWB14 and pKWB15

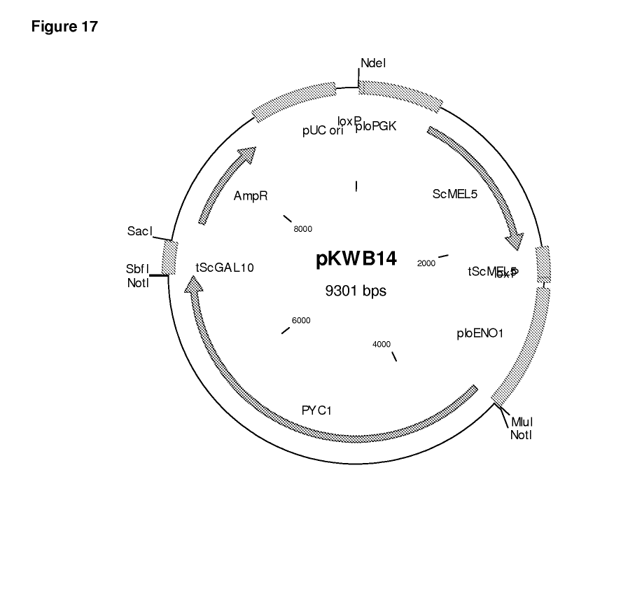

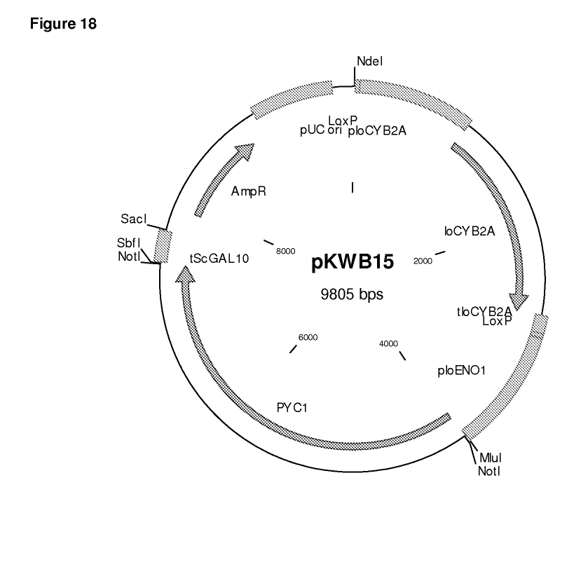

The PYC1 gene from S. cerevisiae (SEQ ID NO:9) is amplified from genomic DNA using Phusion polymerase and primers oKW29 (SEQ ID NO:71) and oKW30 (SEQ ID NO:72), both of which contain at their 5′ end 23 bp flanking the NotI site in pKF031 and pKF044 to enable directional ligation-less cloning. After amplification, the product is gel purified and co-transformed into E. coli with NotI-digested pKF031 and pKF044. Transformants are selected on LB plates containing 100 μg/ml carbenicillin, and screened using primers oKW93 (SEQ ID NO:95) and oKW95 (SEQ ID NO:96). Quickchange PCR is performed using primers oKW81 (SEQ ID NO:89) and oKW82 (SEQ ID NO:90) to eliminate an internal NdeI site (T2838C). Correct plasmids are confirmed by sequencing, and the final constructs are designated pKWB14 (MEL5 marker) (FIG. 17) and pKWB15 (CYB2A marker) (FIG. 18).

### Example 5D

### Construction of K. marxianus PYC1 Expression Constructs PKWB16 and pKWB17

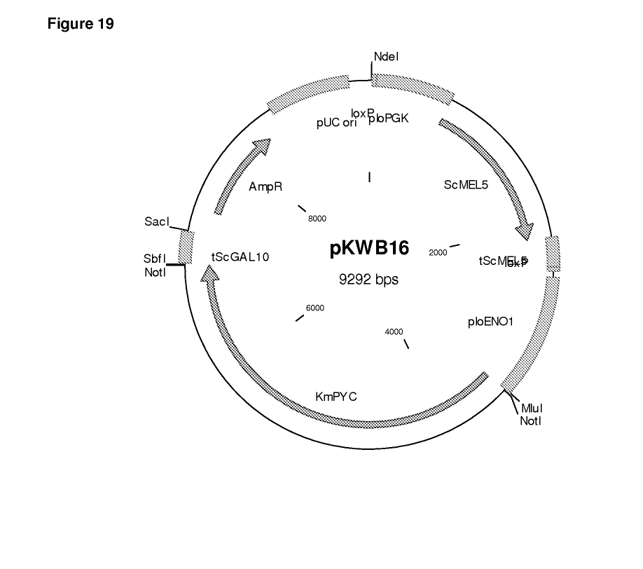

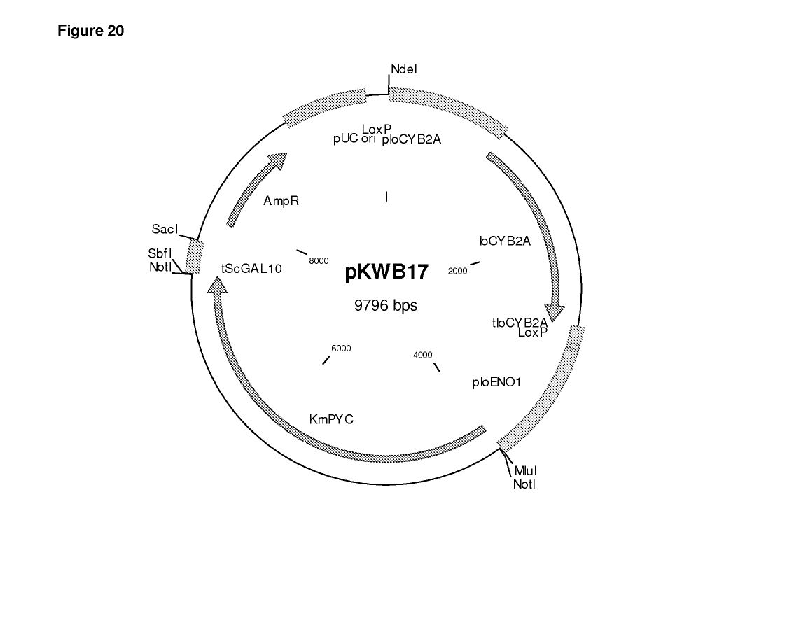

K. marxianus is streaked on YPD plates, and after around 3 days the PYC1 gene (SEQ ID NO:11) is amplified from genomic DNA by colony PCR using primers oKW85 (SEQ ID NO:93) and oKW86 (SEQ ID NO:94). After amplification, the product is gel purified and co-transformed into E. coli with NotI-digested pKF031 and pKF044. Transformants are selected on LB plates containing 100 μg/ml carbenicillin, and screened using primers oKW93 (SEQ ID NO:95) and oKW95 (SEQ ID NO:96). Quickchange PCR is performed using primers oKW83 (SEQ ID NO:91) and oKW84 (SEQ ID NO:92) to eliminate an internal SacI site (T1446A). Correct plasmids are confirmed by sequencing, and the final constructs are designated pKWB16 (contains MEL5 marker) (FIG. 19) and pKWB17 (contains CYB2A marker) (FIG. 20).

### Example 5E

### Insertion of I. orientalis PYC1 at the First and Second PDC1 Loci in I. orientalis Strain 2732

pKF043 and pKF045 are both amplified from the loxP site on the 5′ end to the GAL10 terminator on the 3′ end. pKF043 is amplified using primers oKF243 (SEQ ID NO:115) and oKF244 (SEQ ID NO:116), and pKF045 is amplified using primers oKF255 (SEQ ID NO:120) and oKF244 (SEQ ID NO:116). Each of these primers contains on their 5′ end 65 bp of sequence specific to the 65 bp immediately upstream and downstream of the PDC1 locus in I. orientalis. This recombination sequence enables double recombination and integration at the PDC1 locus.

The PCR product amplified from pKF043 is used to transform I. orientalis strain 2732. Transformants are selected on YNB+melibiose+x-α-gal and, and integration of PYC1 at a first PDC1 allele is confirmed by PCR using primers oCM566 (SEQ ID NO:121), oKF151 (SEQ ID NO:114), oKF252 (SEQ ID NO:119), and oCM587 (SEQ ID NO:122). The correct heterozygous strain is designated SSK10.

Strain SSK10 is transformed with the PCR product from pKF045 amplification to generate a homozygous strain with PYC1 inserted at both PDC1 alleles. Integration is confirmed by PCR using the primers oCM566 (SEQ ID NO:121), oMM174 (SEQ ID NO:51), oCM587 (SEQ ID NO:122), and oCA397 (SEQ ID NO:63). The correct homozygous strain is designated 12339.

For marker recycling, I. orientalis 12339 was grown to around OD₆₀₀ of 1.0 in YP+100 g/L dextrose (50 ml media in a 250 ml flask; 30° C./250 rpm). Cells were transformed with pVB32 using lithium acetate transformation, and transformants were selected on YNB+2% sucrose plates overlaid with x-α-gal. After 4 to 5 days, white colonies were streaked to YP+20 g/L dextrose plates overlaid with x-α-gal and grown at 37° C. for 2 days. Genomic DNA from white colonies was screened for retention of the expression cassette at the I. orientalis PDC1 locus and for loss of the selectable markers using PCR primers oGPB9 (SEQ ID NO:123), oGPB10 (SEQ ID NO:124), oGPB11 (SEQ ID NO:125), and oGPB12 (SEQ ID NO:126). Positive transformants were confirmed to have lost the marker by a phenotypic screen showing no growth on YNB+2% lactic, 2% melabiose, or 2% sucrose. The homozygous strain with both markers removed was designated 12429.

### Example 5F

### Insertion of S. cerevisiae PYC1 at First and Second PDC1 Loci in I. orientalis Strain 12429

pKWB14 and pKWB15 are both digested with NdeI/SacI to liberate the fragment containing the marker cassette, ENO promoter, PYC1 gene, and terminator. These fragments are cloned into pKWB21 digested with NdeI and SacI, and the resultant plasmid is transformed into E. coli. Transformants are selected on LB+kanamycin, and colonies are screened with the primers M13F (SEQ ID NO:134) and M13R (SEQ ID NO:135). Clones having the desired insert are designated pKWB45 (MEL5 marker) and pKWB46 (CYB2B marker).

pKWB45 is digested with PmeI, gel purified, and transformed into I. orientalis strain 12429. Transformants are selected on YNB+lactate or YNB+melibiose+x-α-gal and screened by PCR using flanking primers oKW70 (SEQ ID NO:81) and oKW73 (SEQ ID NO:84) and nested primers oGPB53 (SEQ ID NO:131), oGPB55 (SEQ ID NO:133), oKW121 (SEQ ID NO:108), oKW122 (SEQ ID NO:109) to verify correct insertion at the PDC1 locus. A heterozygous strain with the S. cerevisiae PYC1 gene inserted at a first PDC1 locus is designated ySBCK9.

Strain ySBCK9 is transformed with pKWB46 digested with PmeI, and transformants are screened for integration as above. Strains homozygous for S. cerevisiae PYC1 at the PDC1 loci are designated ySBCK10.

Marker recycling is carried out, and the homozygous strain with both markers removed is designated ySBCK11.

### Example 5G

### Insertion of K. marxianus PYC1 at First and Second PDC1 Loci in I. orientalis Strain 12429

pKWB16 and pKWB17 are both digested with NdeI/SacI to liberate the fragment containing the marker cassette, ENO promoter, PYC gene, and terminator. These fragments are cloned into pKWB21 digested with NdeI and SacI, and the resultant plasmid is transformed into E. coli. Transformants are selected on LB+kanamycin, and colonies are screened with the primers M13F (SEQ ID NO:134) and M13R (SEQ ID NO:135). Clones having the desired insert are designated pKWB47 (MEL5 marker) and pKWB48 (CYB2A marker).

pKWB47 is digested with PmeI, gel purified, and transformed into I. orientalis 12429. Transformants are selected on YNB+lactate or YNB+melibiose and screened by PCR using flanking primers oKW70 (SEQ ID NO:81) and oKW73 (SEQ ID NO:84) and nested primers oGPB53 (SEQ ID NO:131), oGPB55 (SEQ ID NO:133), oKW83 (SEQ ID NO:91), oKW84 (SEQ ID NO:92) to verify correct insertion at the PDC1 locus. A heterozygous strain K. marxianus PYC1 gene inserted at a first PDC1 locus is designated ySBCK12.

Strain ySBCK12 is transformed with pKWB48 digested with PmeI, and transformants are screened for integration as above. Strains homozygous for K. marxianus PYC1 are designated ySBCK13. Marker recycling is carried out, and the homozygous strain with both markers removed is designated ySBCK14.

The various PYC1 insertion/PDC1 deletion strains generated in Example 5 are summarized in Table 7.

TABLE 7
I. orientalis PYC1 insertion strains:

| Strain | | Parent |
|---|---|---|
| name | Description | strain |
| SSK10 | CYB2A deletion (2) | 2732 |
| | GPD1 deletion (2) | |
| | CYB2B deletion (2) | |
| | I. orientalis PYC1 insertion at | |
| | PDC1 (1) | |
| 12339/12429 | CYB2A deletion (2) | SSK10 |
| | GPD1 deletion (2) | |
| | CYB2B deletion (2) | |
| | I. orientalis PYC1 insertion at | |
| | PDC1 (2) | |
| ySBCK9 | CYB2A deletion (2) | 12429 |
| | GPD1 deletion (2) | |
| | CYB2B deletion (2) | |
| | S. cerevisiae PYC1 insertion at | |
| | PDC1 (1) | |
| ySBCK10/ | CYB2A deletion (2) | ySBCK9 |
| ySBCK11 | GPD1 deletion (2) | |
| | CYB2B deletion (2) | |
| | S. cerevisiae PYC1 insertion at | |
| | PDC1 (2) | |
| ySBCK12 | CYB2A deletion (2) | 12429 |
| | GPD1 deletion (2) | |
| | CYB2B deletion (2) | |
| | K. marxianus PYC1 insertion at | |
| | PDC1 (1) | |
| ySBCK13/ | CYB2A deletion (2) | ySBCK12 |
| ySBCK14 | GPD1 deletion (2) | |
| | CYB2B deletion (2) | |
| | K. marxianus PYC1 insertion at | |
| | PDC1 (2) | |

### Example 6

### Insertion of MDH at the ATO2 Locus in I. orientalis Strain 12429

An MDH expression cassette is inserted at one or both ATO2 alleles of I. orientalis strain 12429 (Example 5).

### Example 6A

### Construction of ATO2 Deletion Construct pKWB18

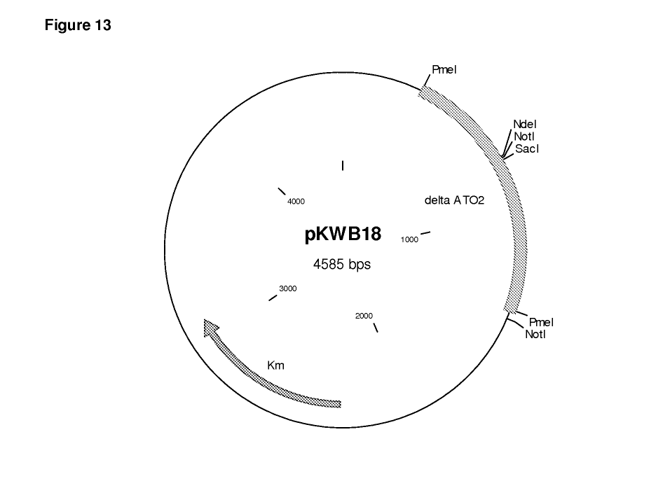

Upstream and downstream regions of I. orientalis ATO2 (SEQ ID NO:45) were amplified in order to generate an ATO2 deletion construct. The upstream and downstream regions correspond to nucleotides from 419 bp upstream to the start codon of ATO2 and from the stop codon to 625 bp downstream, respectively. Amplification of the upstream region is performed using primers oKW66 (forward, SEQ ID NO:77) and oKW67 (reverse, SEQ ID NO:78), which adds a PmeI restriction site and NdeI, NotI, and SacI restriction sites, respectively, to the product. Amplification of the downstream region is performed using primers oKW68 (forward, SEQ ID NO:79) and oKW69 (reverse, SEQ ID NO:80), which adds NdeI, NotI, and SacI restriction sites and a PmeI restriction site, respectively, to the product. The two fragments are amplified independently, then assembled into a full-length product with a two stage PCR protocol. The first stage uses 10 cycles (98° C. 10 s, 55° C. 20 s, 72° C. 60 s) with no primers, and the second stage uses 20 cycles (98° C. 10 s, 55° C. 20 s, 72° C. 60 s) with upstream forward and downstream reverse primers. The full-length product is gel purified, cloned into pCR-BluntII (Invitrogen), and sequenced. The plasmid confirmed to have correct sequence is subjected to quickchange PCR using Phusion polymerase to eliminate the plasmid-borne SacI site. Correct plasmids are confirmed by digestion with SacI and sequencing. The final ATO2 deletion construct is designated pKWB18 (FIG. 13).

### Example 6B

### Construction of I. orientalis MDH Expression Constructs pKWB2-pKWB7

The MDH1, MDH2, and MDH3 genes from I. orientalis (SEQ ID NOs:13, 15, and 17, respectively) are amplified from genomic DNA using primers designed for ligation-less cloning into the NotI site of pKF031 and pKF044. MDH1 is amplified using primers oKW13 (SEQ ID NO:66) and oKW14 (SEQ ID NO:67), MDH2 is amplified using primers oKW15 (SEQ ID NO:68) and oKW16 (SEQ ID NO:69), and MDH3 is amplified using primers oKW114 (SEQ ID NO:105) and oKW18 (SEQ ID NO:70). After amplification, the product is gel purified and co-transformed into E. coli with NotI-digested pKF031 and pKF044. Transformants are selected on LB plates containing 100 μg/ml carbenicillin, and screened using primers oKW93 (SEQ ID NO:95) and oKW95 (SEQ ID NO:96). Correct plasmids are confirmed by sequencing, and the final constructs are designated pKWB2 (MDH1, MEL5 marker), pKWB3 (MDH2, MEL5 marker), pKWB4 (MDH3, MEL5 marker), pKWB5 (MDH1, CYB2A marker), pKWB6 (MDH2, CYB2A marker), and pKWB7 (MDH3, CYB2A marker).

### Example 6C

### Construction of K. marxianus MDH Expression Constructs pKWB8-pKWB13

The MDH1, MDH2, and MDH3 genes from K. marxianus (SEQ ID NOs:19, 21, and 23, respectively) are amplified from genomic DNA using primers designed for ligation-less cloning into the NotI site of pKF031 and pKF044. MDH1 is amplified using primers oKW100 (SEQ ID NO:99) and oKW101 (SEQ ID NO:100), MDH2 is amplified using primers oKW102 (SEQ ID NO:101) and oKW103 (SEQ ID NO:102), and MDH3 is amplified using primers oKW104 (SEQ ID NO:103) and oKW105 (SEQ ID NO:104). After amplification, the product is gel purified and co-transformed into E. coli with NotI-digested pKF031 and pKF044. Transformants are selected on LB plates containing 100 μg/ml carbenicillin, and screened using primers oKW93 (SEQ ID NO:95) and oKW95 (SEQ ID NO:96). Quickchange PCR is performed on MDH2 using primers oKW132 (SEQ ID NO:110), oKW133 (SEQ ID NO:111), oKW134 (SEQ ID NO:112), and oKW135 (SEQ ID NO:113) to eliminate internal SacI sites (G609A and G819A). Quickchange PCR is performed on MDH3 using primers oKW136 and oKW137 to eliminate an internal NdeI site (T18C). Correct plasmids are confirmed by sequencing, and the final constructs are designated pKWB8 (MDH1, MEL5 marker), pKWB9 (MDH2, MEL5 marker), pKWB10 (MDH3, MEL5 marker), pKWB11 (MDH1, MEL5 marker), pKWB12 (MDH2, MEL5 marker), and pKWB13 (MDH3, MEL5 marker).

### Example 6D

### Insertion of I. orientalis MDH1, MDH2, and MDH3 at First and Second ATO2 Loci in I. orientalis Strain 12429

pKWB2, pKWB3, pKWB4, pKWB5, pKWB6, and pKWB7 are digested with NdeI and SacI to liberate the fragment containing selectable marker, ENO promoter, I. orientalis MDH1, 2, or 3, and terminator. These fragments are cloned into NdeI/SacI digested pKWB18 (ATO2 deletion construct), followed by selection on LB+kanamycin. Colonies are screened with M13F (SEQ ID NO:134) and M13R (SEQ ID NO:135) primers to confirm correct clones, which are designated pKWB33 (MDH1, MEL5), pKWB34 (MDH2, MEL5), pKWB35 (MDH3, MEL5), pKWB36 (MDH1, CYB2A), pKWB37 (MDH2, CYB2A), and pKWB38 (MDH3, CYB2A).

pKWB33, pKWB34, and pKWB35 are digested with PmeI and the appropriate fragments transformed into I. orientalis strain 12429 by lithium acetate transformation. Transformants are selected by growth on YNB+lactate or YNB+melibiose and screened by PCR with primers flanking the ATO2 locus (oKW66 (SEQ ID NO:77) and oKW69 (SEQ ID NO:80)) along with nested primers specific to each MDH (oKW13 (SEQ ID NO:66), oKW14 (SEQ ID NO:67), oKW15 (SEQ ID NO:68), oKW16 (SEQ ID NO:69), oKW114 (SEQ ID NO:105), oKW18 (SEQ ID NO:70)). Colonies with the correct insertion of MDH at a first ATO2 locus are designated ySBCK15 (MDH1), ySBCK18 (MDH2), and ySBCK21 (MDH3).

ySBCK15, ySBCK18, and ySBCK21 are transformed with pKWB36, pKWB37, and pKWB38, respectively, and transformants are screened for MDH integration as above. Strains homozygous for I. orientalis MDH at the ATO2 loci are designated ySBCK16 (MDH1), ySBCK19 (MDH2), and ySBCK22 (MDH3).

Marker recycling is carried out using pVB32, and homozygous strains with both markers removed are designated ySBCK17 (MDH1), ySBCK20 (MDH2), and ySBCK23 (MDH3).

### Example 6E

### Insertion of K. marxianus MDH1, MDH2, and MDH3 at First and Second ATO2 Loci in I. orientalis Strain 12429

pKWB8, pKWB9, pKWB10, pKWB11, pKWB12, and pKWB13 are digested with NdeI and SacI to liberate the fragment containing selectable marker, ENO promoter, K. marxianus MDH1, 2, or 3, and terminator. These fragments are cloned into NdeI/SacI digested pKWB18 (ATO2 deletion construct), transformed into E. coli, and selected on LB+kanamycin. Colonies are screened with M13F (SEQ ID NO:134) and M13R (SEQ ID NO:135) primers to confirm correct clones, which are designated pKWB39 (MDH1, MEL5), pKWB40 (MDH2, MEL5), pKWB41 (MDH3, MEL5), pKWB42 (MDH1, CYB2A), pKWB43 (MDH2, CYB2A), pKWB44 (MDH3, CYB2A).

pKWB39, pKWB40, and pKWB41 are digested with PmeI and the appropriate fragments are used to transform I. orientalis strain 12429 by lithium acetate transformation. Transformants are selected by growth on YNB+lactate or YNB+melibiose and screened by PCR with primers flanking the ATO2 locus (oKW66 (SEQ ID NO:77) and oKW69 (SEQ ID NO:80)) along with nested primers specific to each MDH (oKW100 (SEQ ID NO:99), oKW101 (SEQ ID NO:100), oKW102 (SEQ ID NO:101), oKW103 (SEQ ID NO:102), oKW104 (SEQ ID NO:103), oKW105 (SEQ ID NO:104)). Colonies with the correct insertion of MDH at a first ATO2 locus are designated ySBCK24 (MDH1), ySBCK27 (MDH2), and ySBCK30 (MDH3).

pKWB42, pKWB43, and pKWB44 are digested with PmeI and transformed into ySBCK24, ySBCK27, and ySBCK30, respectively, and transformants are screened for MDH integration as above. Strains homozygous for K. marxianus MDH at the ATO2 loci are designated ySBCK25 (MDH1), ySBCK28 (MDH2), and ySBCK31 (MDH3).

Marker recycling is carried out using pVB32, and homozygous strains with both markers removed are designated ySBCK26 (MDH1), ySBCK29 (MDH2), and ySBCK32 (MDH3).

The various MDH insertion/ATO2 deletion strains generated in Example 6 are summarized in Table 8.

TABLE 8
I. orientalis MDH insertion strains:

| Strain | | Parent |
|---|---|---|
| name | Description | strain |
| ySBCK15 | CYB2A deletion (2) | 12429 |
| | GPD1 deletion (2) | |
| | CYB2B deletion (2) | |
| | I. orientalis PYC1 insertion at | |
| | PDC1 (2) | |
| | I. orientalis MDH1 insertion at | |
| | ATO2 (1) | |
| ySBCK16/ | CYB2A deletion (2) | ySBCK15 |
| ySBCK17 | GPD1 deletion (2) | |
| | CYB2B deletion (2) | |
| | I. orientalis PYC1 insertion at | |
| | PDC1 (2) | |
| | I. orientalis MDH1 insertion at | |
| | ATO2 (2) | |
| ySBCK18 | CYB2A deletion (2) | 12429 |
| | GPD1 deletion (2) | |
| | CYB2B deletion (2) | |
| | I. orientalis PYC1 insertion at | |
| | PDC1 (2) | |
| | I. orientalis MDH2 insertion at | |
| | ATO2 (1) | |
| ySBCK19/ | CYB2A deletion (2) | ySBCK18 |
| ySBCK20 | GPD1 deletion (2) | |
| | CYB2B deletion (2) | |
| | I. orientalis PYC1 insertion at | |
| | PDC1 (2) | |
| | I. orientalis MDH2 insertion at | |
| | ATO2 (2) | |
| ySBCK21 | CYB2A deletion (2) | 12429 |
| | GPD1 deletion (2) | |
| | CYB2B deletion (2) | |
| | I. orientalis PYC1 insertion at | |
| | PDC1 (2) | |
| | I. orientalis MDH3 insertion at | |
| | ATO2 (1) | |
| ySBCK22/ | CYB2A deletion (2) | ySBCK21 |
| ySBCK23 | GPD1 deletion (2) | |
| | CYB2B deletion (2) | |
| | I. orientalis PYC1 insertion at | |
| | PDC1 (2) | |
| | I. orientalis MDH3 insertion at | |
| | ATO2 (2) | |
| ySBCK24 | CYB2A deletion (2) | 12429 |
| | GPD1 deletion (2) | |
| | CYB2B deletion (2) | |
| | I. orientalis PYC1 insertion at | |
| | PDC1 (2) | |
| | K. marxianus MDH1 insertion at | |
| | ATO2 (1) | |
| ySBCK25/ | CYB2A deletion (2) | ySBCK24 |
| ySBCK26 | GPD1 deletion (2) | |
| | CYB2B deletion (2) | |
| | I. orientalis PYC1 insertion at | |
| | PDC1 (2) | |
| | K. marxianus MDH1 insertion at | |
| | ATO2 (2) | |
| ySBCK27 | CYB2A deletion (2) | 12429 |
| | GPD1 deletion (2) | |
| | CYB2B deletion (2) | |
| | I. orientalis PYC1 insertion at | |
| | PDC1 (2) | |
| | K. marxianus MDH2 insertion at | |
| | ATO2 (1) | |
| ySBCK28/ | CYB2A deletion (2) | ySBCK27 |
| ySBCK29 | GPD1 deletion (2) | |
| | CYB2B deletion (2) | |
| | I. orientalis PYC1 insertion at | |
| | PDC1 (2) | |
| | K. marxianus MDH2 insertion at | |
| | ATO2 (2) | |
| ySBCK30 | CYB2A deletion (2) | 12429 |
| | GPD1 deletion (2) | |
| | CYB2B deletion (2) | |
| | I. orientalis PYC1 insertion at | |
| | PDC1 (2) | |
| | K. marxianus MDH3 insertion at | |
| | ATO2 (1) | |
| ySBCK31/ | CYB2A deletion (2) | ySBCK30 |
| ySBCK32 | GPD1 deletion (2) | |
| | CYB2B deletion (2) | |
| | I. orientalis PYC1 insertion at | |
| | PDC1 (2) | |
| | K. marxianus MDH3 insertion at | |
| | ATO2 (2) | |

### Example 7

### Insertion of I. orientalis FUM1 Genes at the CYB2a Locus in I. orientalis Strains ySBCK17, 20, 23, 26, 29, and 32

I. orientalis FUM1 expression cassettes are inserted at both alleles of CYB2A in I. orientalis strains ySBCK17, 20, 23, 26, 29, and 32 (Example 6).

### Example 7A

### Construction of I. orientalis FUM1 Expression Constructs DGPB30, DGPB42, pGPB44, and pGPB47

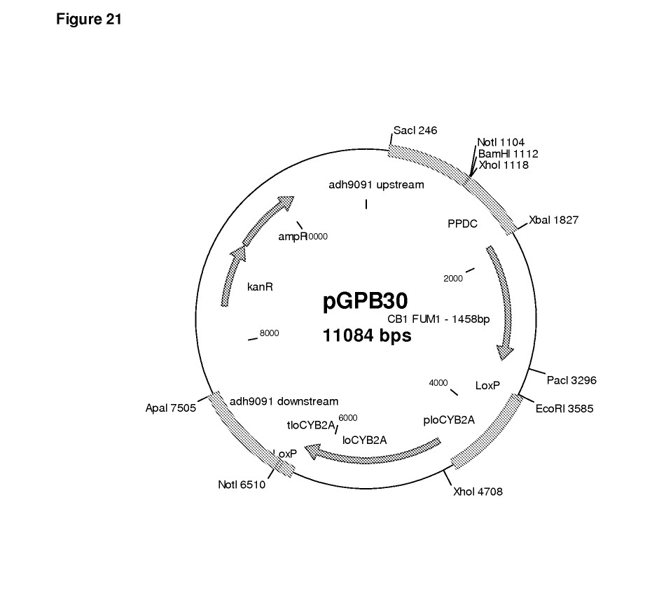

An expression cassette for the I. orientalis FUM1 gene (SEQ ID NO:1) is inserted into the ADH2a deletion construct pGPB11. PCR primers oGPB38 (SEQ ID NO:127) and oGPB40 (SEQ ID NO:128) are used to amplify FUM1 using I. orientalis genomic DNA as the template. The 5′ primer adds an XbaI site at the start site of the coding sequence and the 3′ primer adds a PacI site 3′ of the stop codon. The resulting PCR product is digested with XbaI and PacI and ligated to similarly digested pGPB11. The resulting plasmid, which contains the FUM1 coding sequence flanked by the I. orientalis PDC1 promoter and terminator and the CYB2A selectable marker, is designated pGPB30 (FIG. 21).

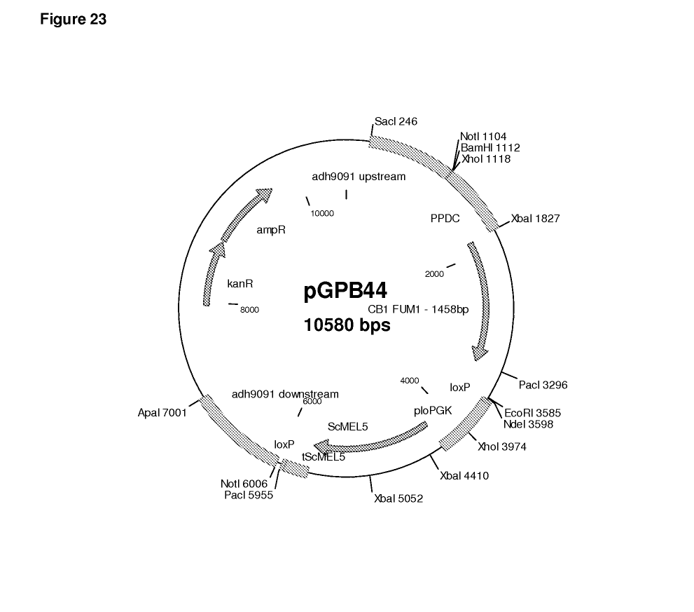

pGPB30 is digested with BamHI and NdeI and ligated into similarly digested pGPB14. The resulting plasmid is designated pGPB44 (FIG. 23).

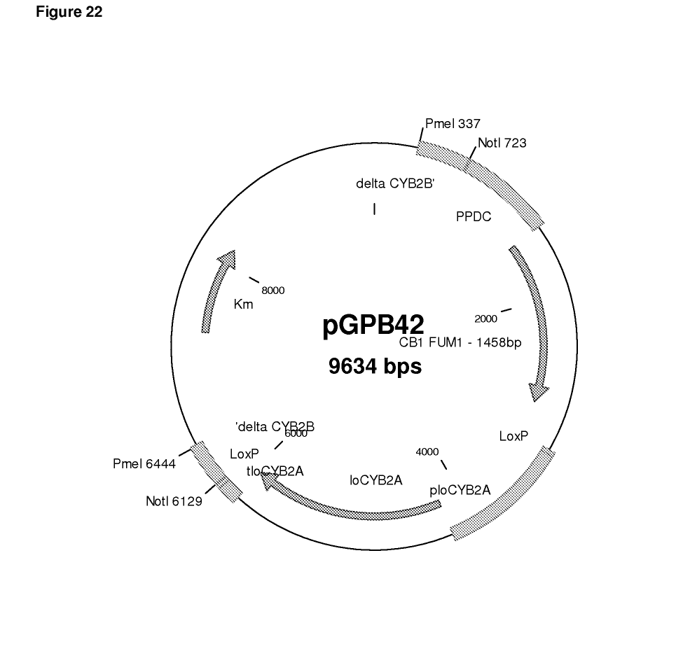

The expression cassette from pGPB30 is excised using NotI and ligated to the NotI cut pKW22. The resulting plasmid is designated pGPB42 (FIG. 22).

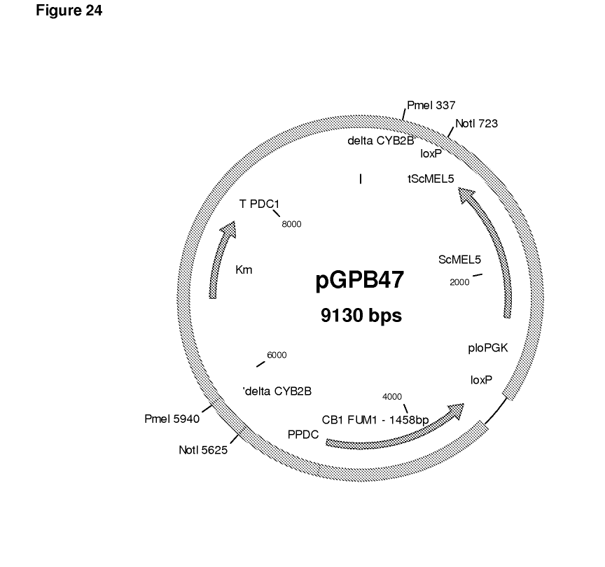

The expression cassette from pGPB44 is excised using NotI and ligated to the NotI cut pKW22. The resulting plasmid is designated pGPB47 (FIG. 24).

### Example 7B

### Insertion of I. orientalis FUM1 at One or Both I. orientalis CYB2B Loci

pGPB42 is digested with SacI and ApaI and transformed into I. orientalis strains ySBCK 17, 20, 23, 26, 29, and 32 using lithium acetate transformation. Transformants are screened by PCR to confirm correct integration of the FUM1 expression cassette at the first CYB2B locus using primers oKW117 (SEQ ID NO:106), oJLJ43 (SEQ ID NO:136), oKW120 (SEQ ID NO:107), and oGPB46 (SEQ ID NO:129). The resulting strains are designated ySBCGH361-ySBCGH366.

Integration of the second copy of the FUM1 expression cassette at the CYB2B locus is performed using plasmids containing the MEL5 selectable marker. pGPB47 is digested with SacI and ApaI and transformed into ySBCGH361-ySBCGH366 using lithium acetate transformation. Transformants are screened by PCR to confirm correct integration of the FUM1 expression cassette at the second CYB2B locus using primers oKW117 (SEQ ID NO:106), oJLJ43 (SEQ ID NO:136), oKW120 (SEQ ID NO:107), and oGPB46 (SEQ ID NO:129). The resulting strains are designated ySBCGH367-ySBCGH372.

Marker recycling is carried out with plasmid pVB32. The correct homozygous strains with both markers removed are designated ySBCGH373-ySBCGH378.

The various FUM1 insertion/CYB2B deletion strains generated in Example 7 are summarized in Table 9.

TABLE 9
I. orientalis FUM1 insertion strains:

| Strain | | Parent |
|---|---|---|
| name | Description | strain |
| ySBCGH361 | CYB2A deletion (2) | ySBCK17 |
| | GPD1 deletion (2) | |
| | CYB2B deletion (2) | |
| | I. orientalis PYC1 insertion at | |
| | PDC1 (2) | |
| | I. orientalis MDH1 insertion at | |
| | ATO2 (2) | |
| | I. orientalis FUM1 insertion at | |
| | CYB2B (1) | |
| ySBCGH367/ | CYB2A deletion (2) | ySBCGH361 |
| ySBCGH373 | GPD1 deletion (2) | |
| | CYB2B deletion (2) | |
| | I. orientalis PYC1 insertion at | |
| | PDC1 (2) | |
| | I. orientalis MDH1 insertion at | |
| | ATO2 (2) | |
| | I. orientalis FUM1 insertion at | |
| | CYB2B (2) | |
| ySBCGH362 | CYB2A deletion (2) | ySBCK20 |
| | GPD1 deletion (2) | |
| | CYB2B deletion (2) | |
| | I. orientalis PYC1 insertion at | |
| | PDC1 (2) | |
| | I. orientalis MDH2 insertion at | |
| | ATO2 (2) | |
| | I. orientalis FUM1 insertion at | |
| | CYB2B (1) | |
| ySBCGH368/ | CYB2A deletion (2) | ySBCGH362 |
| ySBCGH374 | GPD1 deletion (2) | |
| | CYB2B deletion (2) | |
| | I. orientalis PYC1 insertion at | |
| | PDC1 (2) | |
| | I. orientalis MDH2 insertion at | |
| | ATO2 (2) | |
| | I. orientalis FUM1 insertion at | |
| | CYB2B (2) | |
| ySBCGH363 | CYB2A deletion (2) | ySBCK23 |
| | GPD1 deletion (2) | |
| | CYB2B deletion (2) | |
| | I. orientalis PYC1 insertion at | |
| | PDC1 (2) | |
| | I. orientalis MDH3 insertion at | |
| | ATO2 (2) | |
| | I. orientalis FUM1 insertion at | |
| | CYB2B (1) | |
| ySBCGH369/ | CYB2A deletion (2) | ySBCGH363 |
| ySBCGH375 | GPD1 deletion (2) | |
| | CYB2B deletion (2) | |
| | I. orientalis PYC1 insertion at | |
| | PDC1 (2) | |
| | I. orientalis MDH3 insertion at | |
| | ATO2 (2) | |
| | I. orientalis FUM1 insertion at | |
| | CYB2B (2) | |
| ySBCGH364 | CYB2A deletion (2) | ySBCK26 |
| | GPD1 deletion (2) | |
| | CYB2B deletion (2) | |
| | I. orientalis PYC1 insertion at | |
| | PDC1 (2) | |
| | K. marxianus MDH1 insertion at | |
| | ATO2 (2) | |
| | I. orientalis FUM1 insertion at | |
| | CYB2B (1) | |
| ySBCGH370/ | CYB2A deletion (2) | ySBCGH364 |
| ySBCGH376 | GPD1 deletion (2) | |
| | CYB2B deletion (2) | |
| | I. orientalis PYC1 insertion at | |
| | PDC1 (2) | |
| | K. marxianus MDH1 insertion at | |
| | ATO2 (2) | |
| | I. orientalis FUM1 insertion at | |
| | CYB2B (2) | |
| ySBCGH365 | CYB2A deletion (2) | ySBCK29 |
| | GPD1 deletion (2) | |
| | CYB2B deletion (2) | |
| | I. orientalis PYC1 insertion at | |
| | PDC1 (2) | |
| | K. marxianus MDH2 insertion at | |
| | ATO2 (2) | |
| | I. orientalis FUM1 insertion at | |
| | CYB2B (1) | |
| ySBCGH371/ | CYB2A deletion (2) | ySBCGH365 |
| ySBCGH377 | GPD1 deletion (2) | |
| | CYB2B deletion (2) | |
| | I. orientalis PYC1 insertion at | |
| | PDC1 (2) | |
| | K. marxianus MDH2 insertion at | |
| | ATO2 (2) | |
| | I. orientalis FUM1 insertion at | |
| | CYB2B (2) | |
| ySBCGH366 | CYB2A deletion (2) | ySBCK32 |
| | GPD1 deletion (2) | |
| | CYB2B deletion (2) | |
| | I. orientalis PYC1 insertion at | |
| | PDC1 (2) | |
| | K. marxianus MDH3 insertion at | |
| | ATO2 (2) | |
| | I. orientalis FUM1 insertion at | |
| | CYB2B (1) | |
| ySBCGH372/ | CYB2A deletion (2) | ySBCGH366 |
| ySBCGH378 | GPD1 deletion (2) | |
| | CYB2B deletion (2) | |
| | I. orientalis PYC1 insertion at | |
| | PDC1 (2) | |
| | K. marxianus MDH3 insertion at | |
| | ATO2 (2) | |
| | I. orientalis FUM1 insertion at | |
| | CYB2B (2) | |

### Example 8

### Shake Flask Characterization of Malate and Fumarate Production in I. orientalis Strains ySBCK17 and ySBCGH373

Shake flasks are used to test the PYC1/MDH1 insertion strain ySBCK17 (Example 6) and the PYC1/MDH1/FUM insertion strain ySBCGH373 (Example 7). Shake flasks are inoculated with biomass harvested from seed flasks grown overnight to an OD₆₀₀ of 2 to 6. 250 mL baffled flasks (50 mL working volume) are inoculated to an OD₆₀₀ of 0.2 and fermentation occurs at 100 rpm and 30° C. DM defined medium is used in flasks, with pH control and CO₂ provided by calcium carbonate addition at a concentration of 0.255M (1.28 g CaCO₃ per 50 ml flask). Samples are taken throughout the time course of the assay and analyzed for biomass growth via OD₆₀₀, and malate, fumarate, and glucose are monitored via high performance liquid chromatography (HPLC). The resulting data shows production of greater than 40 g/L malate by strain ySBCK17 and production of greater than 40 g/L fumarate by strain ySBCGH373.

### Example 9

### Deletion of the First and Second PCK1 Loci in I. orientalis Strains ySBCK17, 20, 23, 26, 29, and 32 and ySBCGH373-ySBCGH378

The first and second PCK1 loci in I. orientalis strains ySBCK17, 20, 23, 26, 29, and 32 (Example 6) and ySBCGH373-ySBCGH378 (Example 7) are deleted using a PCK deletion construct.

### Example 9A

### Construction of I. orientalis PCK Deletion Constructs

The PCK upstream region from 432 bp upstream to the start codon is amplified by PCR. Sequence corresponding to the restriction sites NdeI/NotI/SacI is added to the 5′ end of the reverse upstream primer (oKW78, SEQ ID NO:86). A PmeI restriction site is added to the 5′ end of the forward upstream primer (oKW77, SEQ ID NO:85). The PCK downstream region is amplified from the stop codon to 472 bp downstream. The downstream forward primer (oKW79, SEQ ID NO:87) contained the same NdeI/NotI/SacI sequence as the reverse upstream primer. The 5′ end of the reverse downstream primer also has a PmeI site (oKW80, SEQ ID NO:88). The two fragments are amplified independently using Phusion polymerase, then assembled into a full-length (926 bp) product via a two-stage PCR protocol (10 cycles (98° C. 10s, 55° C. 20 s, 72° C. 60 s) with no primers, followed by 20 cycles (98° C. 10 s, 55° C. 20 s, 72° C. 60 s) with upstream forward and downstream reverse primers). Full-length product is gel purified and cloned into pCR-BluntII (Invitrogen) and sequenced. The plasmid confirmed to have correct sequence is subjected to quickchange PCR using Phusion polymerase to eliminate the plasmid borne SacI site. Correct plasmids are confirmed by digestion with SacI and sequencing. The final construct is named pKWB20 (FIG. 39).

pKWB20 is digested with NdeI and SacI and the resulting 4.4 kb DNA fragment is gel purified. Plasmid pKF031 is digested with NdeI and SacI to create a fragment of 3.7 kb that contains the MEL5 marker flanked by loxP sites. In the same way, pKF044 is digested to create a 4.2 kb fragment containing the CYB2A marker flanked by loxP sites. Marker fragments are ligated into the digested pKWB20 plasmid to create pKWB25 (FIG. 40), containing the MEL5 marker, and pKWB30 (FIG. 41), containing the CYB2A marker. Correct constructs are confirmed by PCR and restriction digestion.

### Example 9B

### Deletion of PCK1 in I. orientalis Strains ySBCK 17, 20, 23, 26, 29, and 32 and ySBCGH373-ySBCGH378

Plasmid pKWB25 is digested with PmeI to create a 5 kb fragment containing the MEL5 marker surrounded by PCK1 flanking sequence. The fragment is gel purified prior to transformation. In the same way, pKWB30 is digested with PmeI to create a 5.4 kb fragment containing the CYB2A marker with PCK1 flanking sequence. The fragment is gel purified prior to transformation.

The DNA fragment from pKWB30, containing the CYB2A marker, is transformed into strains ySBCK 17, 20, 23, 26, 29, and 32 and ySBCGH373-ySBCGH378. Transformants are selected on YNB+lactate, and deletion of PCK at the first allele is confirmed by PCR using primers oKW77 (SEQ ID NO:85), oKW80 (SEQ ID NO:88), oGPB52 (SEQ ID NO:130), and oGPB53 (SEQ ID NO:131). The correct heterozygous strains are designated ySBCGH379-384 and ySBCGH397-402.

Strains ySBCGH379-384 and ySBCGH397-402 are transformed with the PmeI digestion product from pKWB30 and selected on YNB+melibiose+x-α-gal to generate homozygous strains with PCK deleted at both alleles. Integration is confirmed by PCR using the primers oKW77 (SEQ ID NO:85), oKW80 (SEQ ID NO:88), oGPB54 (SEQ ID NO:132), and oGPB55 (SEQ ID NO:133). The correct homozygous strains are designated ySBCGH385-390 and ySBCGH403-408.

Marker recycling is carried out with plasmid pVB32. The correct homozygous strains with both markers removed are designated ySBCGH391-396 and ySBCGH409-414.

The various PCK deletion strains generated in Example 9 are summarized in Table 10.

TABLE 10
I. orientalis PCK deletion strains:

| Strain | | Parent |
|---|---|---|
| name | Description | strain |
| ySBCGH379 | CYB2A deletion (2) | ySBCK17 |
| | GPD1 deletion (2) | |
| | CYB2B deletion (2) | |
| | PCK1 deletion (1) | |
| | I. orientalis PYC1 insertion at | |
| | PDC1 (2) | |
| | I. orientalis MDH1 insertion at | |
| | ATO2 (2) | |
| ySBCGH385/ | CYB2A deletion (2) | ySBCGH379 |
| ySBCGH391 | GPD1 deletion (2) | |
| | CYB2B deletion (2) | |
| | PCK1 deletion (2) | |
| | I. orientalis PYC1 insertion at | |
| | PDC1 (2) | |
| | I. orientalis MDH1 insertion at | |
| | ATO2 (2) | |
| ySBCGH380 | CYB2A deletion (2) | ySBCK20 |
| | GPD1 deletion (2) | |
| | CYB2B deletion (2) | |
| | PCK1 deletion (1) | |
| | I. orientalis PYC1 insertion at | |
| | PDC1 (2) | |
| | I. orientalis MDH2 insertion at | |
| | ATO2 (2) | |
| ySBCGH386/ | CYB2A deletion (2) | ySBCGH380 |
| ySBCGH392 | GPD1 deletion (2) | |
| | CYB2B deletion (2) | |
| | PCK1 deletion (2) | |
| | I. orientalis PYC1 insertion at | |
| | PDC1 (2) | |
| | I. orientalis MDH2 insertion at | |
| | ATO2 (2) | |
| ySBCGH381 | CYB2A deletion (2) | ySBCK23 |
| | GPD1 deletion (2) | |
| | CYB2B deletion (2) | |
| | PCK1 deletion (1) | |
| | I. orientalis PYC1 insertion at | |
| | PDC1 (2) | |
| | I. orientalis MDH3 insertion at | |
| | ATO2 (2) | |
| ySBCGH387/ | CYB2A deletion (2) | ySBCGH381 |
| ySBCGH393 | GPD1 deletion (2) | |
| | CYB2B deletion (2) | |
| | PCK1 deletion (2) | |
| | I. orientalis PYC1 insertion at | |
| | PDC1 (2) | |
| | I. orientalis MDH3 insertion at | |
| | ATO2 (2) | |
| ySBCGH382 | CYB2A deletion (2) | ySBCK26 |
| | GPD1 deletion (2) | |
| | CYB2B deletion (2) | |
| | PCK1 deletion (1) | |
| | I. orientalis PYC1 insertion at | |
| | PDC1 (2) | |
| | K. marxianus MDH1 insertion at | |
| | ATO2 (2) | |
| ySBCGH388/ | CYB2A deletion (2) | ySBCGH382 |
| ySBCGH394 | GPD1 deletion (2) | |
| | CYB2B deletion (2) | |
| | PCK1 deletion (2) | |
| | I. orientalis PYC1 insertion at | |
| | PDC1 (2) | |
| | K. marxianus MDH1 insertion at | |
| | ATO2 (2) | |
| ySBCGH383 | CYB2A deletion (2) | ySBCK29 |
| | GPD1 deletion (2) | |
| | CYB2B deletion (2) | |
| | PCK1 deletion (1) | |
| | I. orientalis PYC1 insertion at | |
| | PDC1 (2) | |
| | K. marxianus MDH2 insertion at | |
| | ATO2 (2) | |
| ySBCGH389/ | CYB2A deletion (2) | ySBCGH383 |
| ySBCGH395 | GPD1 deletion (2) | |
| | CYB2B deletion (2) | |
| | PCK1 deletion (2) | |
| | I. orientalis PYC1 insertion at | |
| | PDC1 (2) | |
| | K. marxianus MDH2 insertion at | |
| | ATO2 (2) | |
| ySBCGH384 | CYB2A deletion (2) | ySBCK32 |
| | GPD1 deletion (2) | |
| | CYB2B deletion (2) | |
| | PCK1 deletion (1) | |
| | I. orientalis PYC1 insertion at | |
| | PDC1 (2) | |
| | K. marxianus MDH3 insertion at | |
| | ATO2 (2) | |
| ySBCGH390/ | CYB2A deletion (2) | ySBCGH384 |
| ySBCGH396 | GPD1 deletion (2) | |
| | CYB2B deletion (2) | |
| | PCK1 deletion (2) | |
| | I. orientalis PYC1 insertion at | |
| | PDC1 (2) | |
| | K. marxianus MDH3 insertion at | |
| | ATO2 (2) | |
| ySBCGH397 | CYB2A deletion (2) | ySBCGH373 |
| | GPD1 deletion (2) | |
| | CYB2B deletion (2) | |
| | PCK1 deletion (1) | |
| | I. orientalis PYC1 insertion at | |
| | PDC1 (2) | |
| | I. orientalis MDH1 insertion at | |
| | ATO2 (2) | |
| | I. orientalis FUM1 insertion at | |
| | CYB2B (2) | |
| ySBCGH403/ | CYB2A deletion (2) | ySBCGH397 |
| ySBCGH409 | GPD1 deletion (2) | |
| | CYB2B deletion (2) | |
| | PCK1 deletion (2) | |
| | I. orientalis PYC1 insertion at | |
| | PDC1 (2) | |
| | I. orientalis MDH1 insertion at | |
| | ATO2 (2) | |
| | I. orientalis FUM1 insertion at | |
| | CYB2B (2) | |
| ySBCGH398 | CYB2A deletion (2) | ySBCGH374 |
| | GPD1 deletion (2) | |
| | CYB2B deletion (2) | |
| | PCK1 deletion (1) | |
| | I. orientalis PYC1 insertion at | |
| | PDC1 (2) | |
| | I. orientalis MDH2 insertion at | |
| | ATO2 (2) | |
| | I. orientalis FUM1 insertion at | |
| | CYB2B (2) | |
| ySBCGH404/ | CYB2A deletion (2) | ySBCGH398 |
| ySBCGH410 | GPD1 deletion (2) | |
| | CYB2B deletion (2) | |
| | PCK1 deletion (2) | |
| | I. orientalis PYC1 insertion at | |
| | PDC1 (2) | |
| | I. orientalis MDH2 insertion at | |
| | ATO2 (2) | |
| | I. orientalis FUM1 insertion at | |
| | CYB2B (2) | |
| ySBCGH399 | CYB2A deletion (2) | ySBCGH375 |
| | GPD1 deletion (2) | |
| | CYB2B deletion (2) | |
| | PCK1 deletion (1) | |
| | I. orientalis PYC1 insertion at | |
| | PDC1 (2) | |
| | I. orientalis MDH3 insertion at | |
| | ATO2 (2) | |
| | I. orientalis FUM1 insertion at | |
| | CYB2B (2) | |
| ySBCGH405/ | CYB2A deletion (2) | ySBCGH399 |
| ySBCGH411 | GPD1 deletion (2) | |
| | CYB2B deletion (2) | |
| | PCK1 deletion (2) | |
| | I. orientalis PYC1 insertion at | |
| | PDC1 (2) | |
| | I. orientalis MDH3 insertion at | |
| | ATO2 (2) | |
| | I. orientalis FUM1 insertion at | |
| | CYB2B (2) | |
| ySBCGH400 | CYB2A deletion (2) | ySBCGH376 |
| | GPD1 deletion (2) | |
| | CYB2B deletion (2) | |
| | PCK1 deletion (1) | |
| | I. orientalis PYC1 insertion at | |
| | PDC1 (2) | |
| | K. marxianus MDH1 insertion at | |
| | ATO2 (2) | |
| | I. orientalis FUM1 insertion at | |
| | CYB2B (2) | |
| ySBCGH406/ | CYB2A deletion (2) | ySBCGH400 |
| ySBCGH412 | GPD1 deletion (2) | |
| | CYB2B deletion (2) | |
| | PCK1 deletion (2) | |
| | I. orientalis PYC1 insertion at | |
| | PDC1 (2) | |
| | K. marxianus MDH1 insertion at | |
| | ATO2 (2) | |
| | I. orientalis FUM1 insertion at | |
| | CYB2B (2) | |
| ySBCGH401 | CYB2A deletion (2) | ySBCGH377 |
| | GPD1 deletion (2) | |
| | CYB2B deletion (2) | |
| | PCK1 deletion (1) | |
| | I. orientalis PYC1 insertion at | |
| | PDC1 (2) | |
| | K. marxianus MDH2 insertion at | |
| | ATO2 (2) | |
| | I. orientalis FUM1 insertion at | |
| | CYB2B (2) | |
| ySBCGH407/ | CYB2A deletion (2) | ySBCGH401 |
| ySBCGH413 | GPD1 deletion (2) | |
| | CYB2B deletion (2) | |
| | PCK1 deletion (2) | |
| | I. orientalis PYC1 insertion at | |
| | PDC1 (2) | |
| | K. marxianus MDH2 insertion at | |
| | ATO2 (2) | |
| | I. orientalis FUM1 insertion at | |
| | CYB2B (2) | |
| ySBCGH402 | CYB2A deletion (2) | ySBCGH378 |
| | GPD1 deletion (2) | |
| | CYB2B deletion (2) | |
| | PCK1 deletion (1) | |
| | I. orientalis PYC1 insertion at | |
| | PDC1 (2) | |
| | K. marxianus MDH3 insertion at | |
| | ATO2 (2) | |
| | I. orientalis FUM1 insertion at | |
| | CYB2B (2) | |
| ySBCGH408/ | CYB2A deletion (2) | ySBCGH402 |
| ySBCGH414 | GPD1 deletion (2) | |
| | CYB2B deletion (2) | |
| | PCK1 deletion (2) | |
| | I. orientalis PYC1 insertion at | |
| | PDC1 (2) | |
| | K. marxianus MDH3 insertion at | |
| | ATO2 (2) | |
| | I. orientalis FUM1 insertion at | |
| | CYB2B (2) | |

### Example 10

### Deletion of the First and Second MAE1 Loci in I. orientalis Strains ySBCK17, 20, 23, 26, 29, and 32 and ySBCGH373-ySBCGH378

The first and second MAE1 loci in I. orientalis strains ySBCK17, 20, 23, 26, 29, and 32 (Example 6) and ySBCGH373-ySBCGH378 (Example 7) are deleted using an MAE1 deletion construct.

### Example 10A

### Construction of I. Orientalis MAE1 Deletion Constructs

The MAE1 upstream region from 370 bp upstream to the start codon is amplified by PCR. Sequence corresponding to the restriction sites NdeI/NotI/SacI is added to the 5′ end of the reverse upstream primer (oKW61, SEQ ID NO:74). A PmeI restriction site is added to the 5′ end of the forward upstream primer (oKW60, SEQ ID NO:73). The MAE1 downstream region is amplified from the stop codon to 392 bp downstream. The downstream forward primer (oKW62, SEQ ID NO:75) contains the same NdeI/NotI/SacI sequence as the reverse upstream primer. The 5′ end of the reverse downstream primer also has a PmeI site (oKW63, SEQ ID NO:76). The two fragments are amplified independently using Phusion polymerase, then assembled into a full-length (784 bp) product via a two-stage PCR protocol (10 cycles (98° C. 10 s, 55° C. 20 s, 72° C. 60 s) with no primers, followed by 20 cycles (98° C. 10 s, 55° C. 20 s, 72° C. 60 s) with upstream forward and downstream reverse primers). Full-length product is gel purified and cloned into pCR-BluntII (Invitrogen) and sequenced. The plasmid confirmed to have correct sequence is subjected to quickchange PCR using Phusion polymerase to eliminate the plasmid borne SacI site. Correct plasmids are confirmed by digestion with SacI and sequencing. The final construct is named pKWB19 (FIG. 36).

pKWB19 is digested with NdeI and SacI and the resulting 4.3 kb DNA fragment is gel purified. Plasmid pKF031 is digested with NdeI and SacI to create a fragment of 3.7 kb that contains the MEL5 marker flanked by loxP sites. In the same way, pKF044 is digested to create a 4.2 kb fragment containing the CYB2A marker flanked by loxP sites. Marker fragments are ligated into the digested pKWB19 plasmid to create pKWB24 (FIG. 37), containing the MEL5 marker, and pKWB29 (FIG. 38), containing the CYB2A marker. Correct constructs are confirmed by PCR and restriction digestion.

### Example 10B

### Deletion of MAE1 in I. orientalis Strains ySBCK 17, 20, 23, 26, 29, and 32 and ySBCGH373-ySBCGH378

Plasmid pKWB24 is digested with PmeI to create a 4.4 kb fragment containing the MEL5 marker surrounded by MAE flanking sequence. The fragment is gel purified prior to transformation. In the same way, pKWB29 is digested with PmeI to create a 5 kb fragment containing the CYB2A marker with MAE flanking sequence. The fragment is gel purified prior to transformation.

The DNA fragment containing the CYB2A marker is transformed into strains ySBCK 17, 20, 23, 26, 29, and 32 and ySBCGH373-ySBCGH378. Transformants are selected on YNB+lactate, and deletion of MAE at the first allele is confirmed by PCR using primers oKW60 (SEQ ID NO:73), oKW63 (SEQ ID NO:76), oGPB52 (SEQ ID NO:130), and oGPB53 (SEQ ID NO:131). The correct heterozygous strains are designated ySBCGH415-420 and ySBCGH433-438.

Strains ySBCGH415-420 and ySBCGH433-438 are transformed with the PmeI digestion product from pKWB24 and selected on YNB+melibiose+x-α-gal to generate homozygous strains with MAE deleted at both alleles. Integration is confirmed by PCR using the primers oKW60 (SEQ ID NO:73), oKW63 (SEQ ID NO:76), oGPB54 (SEQ ID NO:132), and oGPB55 (SEQ ID NO:133). The correct homozygous strains are designated ySBCGH421-426 and ySBCGH439-444.

Marker recycling is carried out with plasmid pVB32. The correct homozygous strains with both markers removed are designated ySBCGH427-432 and ySBCGH445-450.

The various MAE deletion strains generated in Example 10 are summarized in Table 11.

TABLE 11
I. orientalis MAE deletion strains:

| Strain | | Parent |
|---|---|---|
| name | Description | strain |
| ySBCGH415 | CYB2A deletion (2) | ySBCK17 |
| | GPD1 deletion (2) | |
| | CYB2B deletion (2) | |
| | MAE1 deletion (1) | |
| | I. orientalis PYC1 insertion at | |
| | PDC1 (2) | |
| | I. orientalis MDH1 insertion at | |
| | ATO2 (2) | |
| ySBCGH421/ | CYB2A deletion (2) | ySBCGH415 |
| ySBCGH427 | GPD1 deletion (2) | |
| | CYB2B deletion (2) | |
| | MAE1 deletion (2) | |
| | I. orientalis PYC1 insertion at | |
| | PDC1 (2) | |
| | I. orientalis MDH1 insertion at | |
| | ATO2 (2) | |
| ySBCGH416 | CYB2A deletion (2) | ySBCK20 |
| | GPD1 deletion (2) | |
| | CYB2B deletion (2) | |
| | MAE1 deletion (1) | |
| | I. orientalis PYC1 insertion at | |
| | PDC1 (2) | |
| | I. orientalis MDH2 insertion at | |
| | ATO2 (2) | |
| ySBCGH422/ | CYB2A deletion (2) | ySBCGH416 |
| ySBCGH428 | GPD1 deletion (2) | |
| | CYB2B deletion (2) | |
| | MAE1 deletion (2) | |
| | I. orientalis PYC1 insertion at | |
| | PDC1 (2) | |
| | I. orientalis MDH2 insertion at | |
| | ATO2 (2) | |
| ySBCGH417 | CYB2A deletion (2) | ySBCK23 |
| | GPD1 deletion (2) | |
| | CYB2B deletion (2) | |
| | MAE1 deletion (1) | |
| | I. orientalis PYC1 insertion at | |
| | PDC1 (2) | |
| | I. orientalis MDH3 insertion at | |
| | ATO2 (2) | |
| ySBCGH423/ | CYB2A deletion (2) | ySBCGH417 |
| ySBCGH429 | GPD1 deletion (2) | |
| | CYB2B deletion (2) | |
| | MAE1 deletion (2) | |
| | I. orientalis PYC1 insertion at | |
| | PDC1 (2) | |
| | I. orientalis MDH3 insertion at | |
| | ATO2 (2) | |
| ySBCGH418 | CYB2A deletion (2) | ySBCK26 |
| | GPD1 deletion (2) | |
| | CYB2B deletion (2) | |
| | MAE1 deletion (1) | |
| | I. orientalis PYC1 insertion at | |
| | PDC1 (2) | |
| | K. marxianus MDH1 insertion at | |
| | ATO2 (2) | |
| ySBCGH424/ | CYB2A deletion (2) | ySBCGH418 |
| ySBCGH430 | GPD1 deletion (2) | |
| | CYB2B deletion (2) | |
| | MAE1 deletion (2) | |
| | I. orientalis PYC1 insertion at | |
| | PDC1 (2) | |
| | K. marxianus MDH1 insertion at | |
| | ATO2 (2) | |
| ySBCGH419 | CYB2A deletion (2) | ySBCK29 |
| | GPD1 deletion (2) | |
| | CYB2B deletion (2) | |
| | MAE1 deletion (1) | |
| | I. orientalis PYC1 insertion at | |
| | PDC1 (2) | |
| | K. marxianus MDH2 insertion at | |
| | ATO2 (2) | |
| ySBCGH425/ | CYB2A deletion (2) | ySBCGH419 |
| ySBCGH431 | GPD1 deletion (2) | |
| | CYB2B deletion (2) | |
| | MAE1 deletion (2) | |
| | I. orientalis PYC1 insertion at | |
| | PDC1 (2) | |
| | K. marxianus MDH2 insertion at | |
| | ATO2 (2) | |
| ySBCGH420 | CYB2A deletion (2) | ySBCK32 |
| | GPD1 deletion (2) | |
| | CYB2B deletion (2) | |
| | MAE1 deletion (1) | |
| | I. orientalis PYC1 insertion at | |
| | PDC1 (2) | |
| | K. marxianus MDH3 insertion at | |
| | ATO2 (2) | |
| ySBCGH426/ | CYB2A deletion (2) | ySBCGH420 |
| ySBCGH432 | GPD1 deletion (2) | |
| | CYB2B deletion (2) | |
| | MAE1 deletion (2) | |
| | I. orientalis PYC1 insertion at | |
| | PDC1 (2) | |
| | K. marxianus MDH3 insertion at | |
| | ATO2 (2) | |
| ySBCGH433 | CYB2A deletion (2) | ySBCGH373 |
| | GPD1 deletion (2) | |
| | CYB2B deletion (2) | |
| | MAE1 deletion (1) | |
| | I. orientalis PYC1 insertion at | |
| | PDC1 (2) | |
| | I. orientalis MDH1 insertion at | |
| | ATO2 (2) | |
| | I. orientalis FUM1 insertion at | |
| | CYB2B (2) | |
| ySBCGH439/ | CYB2A deletion (2) | ySBCGH433 |
| ySBCGH445 | GPD1 deletion (2) | |
| | CYB2B deletion (2) | |
| | MAE1 deletion (2) | |
| | I. orientalis PYC1 insertion at | |
| | PDC1 (2) | |
| | I. orientalis MDH1 insertion at | |
| | ATO2 (2) | |
| | I. orientalis FUM1 insertion at | |
| | CYB2B (2) | |
| ySBCGH434 | CYB2A deletion (2) | ySBCGH374 |
| | GPD1 deletion (2) | |
| | CYB2B deletion (2) | |
| | MAE1 deletion (1) | |
| | I. orientalis PYC1 insertion at | |
| | PDC1 (2) | |
| | I. orientalis MDH2 insertion at | |
| | ATO2 (2) | |
| | I. orientalis FUM1 insertion at | |
| | CYB2B (2) | |
| ySBCGH440/ | CYB2A deletion (2) | ySBCGH434 |
| ySBCGH446 | GPD1 deletion (2) | |
| | CYB2B deletion (2) | |
| | MAE1 deletion (2) | |
| | I. orientalis PYC1 insertion at | |
| | PDC1 (2) | |
| | I. orientalis MDH2 insertion at | |
| | ATO2 (2) | |
| | I. orientalis FUM1 insertion at | |
| | CYB2B (2) | |
| ySBCGH435 | CYB2A deletion (2) | ySBCGH375 |
| | GPD1 deletion (2) | |
| | CYB2B deletion (2) | |
| | MAE1 deletion (1) | |
| | I. orientalis PYC1 insertion at | |
| | PDC1 (2) | |
| | I. orientalis MDH3 insertion at | |
| | ATO2 (2) | |
| | I. orientalis FUM1 insertion at | |
| | CYB2B (2) | |
| ySBCGH441/ | CYB2A deletion (2) | ySBCGH435 |
| ySBCGH447 | GPD1 deletion (2) | |
| | CYB2B deletion (2) | |
| | MAE1 deletion (2) | |
| | I. orientalis PYC1 insertion at | |
| | PDC1 (2) | |
| | I. orientalis MDH3 insertion at | |
| | ATO2 (2) | |
| | I. orientalis FUM1 insertion at | |
| | CYB2B (2) | |
| ySBCGH436 | CYB2A deletion (2) | ySBCGH376 |
| | GPD1 deletion (2) | |
| | CYB2B deletion (2) | |
| | MAE1 deletion (1) | |
| | I. orientalis PYC1 insertion at | |
| | PDC1 (2) | |
| | K. marxianus MDH1 insertion at | |
| | ATO2 (2) | |
| | I. orientalis FUM1 insertion at | |
| | CYB2B (2) | |
| ySBCGH442/ | CYB2A deletion (2) | ySBCGH436 |
| ySBCGH448 | GPD1 deletion (2) | |
| | CYB2B deletion (2) | |
| | MAE1 deletion (2) | |
| | I. orientalis PYC1 insertion at | |
| | PDC1 (2) | |
| | K. marxianus MDH1 insertion at | |
| | ATO2 (2) | |
| | I. orientalis FUM1 insertion at | |
| | CYB2B (2) | |
| ySBCGH437 | CYB2A deletion (2) | ySBCGH377 |
| | GPD1 deletion (2) | |
| | CYB2B deletion (2) | |
| | MAE1 deletion (1) | |
| | I. orientalis PYC1 insertion at | |
| | PDC1 (2) | |
| | K. marxianus MDH2 insertion at | |
| | ATO2 (2) | |
| | I. orientalis FUM1 insertion at | |
| | CYB2B (2) | |
| ySBCGH443/ | CYB2A deletion (2) | ySBCGH437 |
| ySBCGH449 | GPD1 deletion (2) | |
| | CYB2B deletion (2) | |
| | MAE1 deletion (2) | |
| | I. orientalis PYC1 insertion at | |
| | PDC1 (2) | |
| | K. marxianus MDH2 insertion at | |
| | ATO2 (2) | |
| | I. orientalis FUM1 insertion at | |
| | CYB2B (2) | |
| ySBCGH438 | CYB2A deletion (2) | ySBCGH378 |
| | GPD1 deletion (2) | |
| | CYB2B deletion (2) | |
| | MAE1 deletion (1) | |
| | I. orientalis PYC1 insertion at | |
| | PDC1 (2) | |
| | K. marxianus MDH3 insertion at | |
| | ATO2 (2) | |
| | I. orientalis FUM1 insertion at | |
| | CYB2B (2) | |
| ySBCGH444/ | CYB2A deletion (2) | ySBCGH438 |
| ySBCGH450 | GPD1 deletion (2) | |
| | CYB2B deletion (2) | |
| | MAE1 deletion (2) | |
| | I. orientalis PYC1 insertion at | |
| | PDC1 (2) | |
| | K. marxianus MDH3 insertion at | |
| | ATO2 (2) | |
| | I. orientalis FUM1 insertion at | |
| | CYB2B (2) | |

1. A genetically modified yeast cell from the Pichia fermentans/Isssatchenkia orientalis clade having an active malate fermentation pathway from phosphoenolpyruvate or pyruvate to malate, wherein the active malate fermentation pathway includes a reaction (a) pyruvate to oxaloacetate, and wherein one or more genes that encode an enzyme for reaction (a) is selected from the group consisting of: (i) an exogenous gene, (ii) an endogenous gene operatively linked to an exogenous regulatory element, and (iii) mixtures thereof.

2. The genetically modified yeast cell of claim 1, wherein the active malate fermentation pathway further includes the reaction
oxaloacetate to malate.

3. The genetically modified yeast cell of claim 2, wherein the cell comprises one or more endogenous genes that encode an enzyme that catalyzes reaction (b).

4. The genetically modified yeast cell of claim 3, wherein the cell comprises one or more copies of one or more endogenous genes that encode for malate dehydrogenase, wherein the endogenous genes are operatively liked to an exogenous regulatory element.

5. The genetically modified yeast cell of claim 4, wherein one or more of said exogenous regulatory elements is selected from the group consisting of an exogenous promoter and an exogenous terminator.

6. The genetically modified yeast cell of claim 2, wherein the cell comprises one or more exogenous genes that encode an enzyme that catalyzes reaction (b).

7. (canceled)

8. The genetically modified yeast cell of claim 1, having an active fumarate fermentation pathway from phosphoenolpyruvate or pyruvate to fumarate.

9. The genetically modified yeast cell of claim 8, wherein the active fumarate fermentation pathway includes the reactions:
(a) oxaloacetate to malate; and
(b) malate to fumarate.

10. (canceled)

11. The genetically modified yeast cell of claim 9, wherein the cell comprises one or more copies of one or more endogenous genes selected from the group consisting of malate dehydrogenase, and fumarase.

12. The genetically modified yeast cell of claim 11, wherein one or more of said endogenous genes are operatively linked to an exogenous regulatory element selected from the group consisting of an exogenous promoter and an exogenous terminator.

13. The genetically modified yeast cell of claim 9, wherein the cell comprises one or more exogenous genes that encode an enzyme that catalyzes any of reactions (b) through (c).

14. The genetically modified yeast cell of claim 13, wherein the cell comprises one or more copies of one or more exogenous genes that encode for pyruvate carboxylase.

15. (canceled)

16. The genetically modified yeast cell of claim 14, wherein said exogenous pyruvate carboxylase gene encodes a polypeptide comprising an amino acid sequence with at least 95% sequence identity to the amino acid sequence set forth in SEQ ID NO:8, SEQ ID NO:10, or SEQ ID NO:12.

17-28. (canceled)

29. The genetically modified yeast cell of claim 13, wherein the cell comprises one or more exogenous fumarase genes that encode for fumarase, and wherein the exogenous fumarase gene encodes a polypeptide comprising an amino acid sequence with at least 95% sequence identity to the amino acid sequence set forth in SEQ ID NO:2.

30-63. (canceled)

64. The genetically modified yeast cell of claim 1, wherein the yeast cell is malate resistant.

65. The genetically modified yeast cell of claim 1, wherein the yeast cell is fumarate resistant.

66. A method of producing malate comprising culturing the genetically modified yeast cell of claim 1 in the presence of at least one carbon source and isolating malate from the culture.

67. A method of producing fumarate comprising culturing the genetically modified yeast cell of claim 8 in the presence of at least one carbon source and isolating fumarate from the culture.

68. The method of claim 66, wherein said carbon source is selected from the group consisting of glucose, xylose, arabinose, sucrose, fructose, cellulose, glucose oligomers, and glycerol.

69. The method of claim 67, wherein said carbon source is selected from the group consisting of glucose, xylose, arabinose, sucrose, fructose, cellulose, glucose oligomers, and glycerol.

70. The genetically modified yeast cell of claim 1, wherein the active malate fermentation pathway further includes:
(d) export of malate from inside the cell to the extracellular environment.

71. The genetically modified yeast cell of claim 8, wherein the active fumarate fermentation pathway further includes:
(d) export of fumarate from inside the cell to the extracellular environment.
